(19) United States
(12) Patent Application Publication
(10) Pub. No.: US 2010/0022483 A1
(43) Pub. Date: Jan. 28, 2010

(54) Substituted Tetracycline Compounds

(75) Inventors: Joel Berniac, Stoneham, MA (US); Todd Bowser, Charlton, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Paratek Pharmaceuticals, Inc., Boston, MA (US)

(21) Appl. No.: 12/423,718

(22) Filed: Apr. 14, 2009

Related U.S. Application Data
(60) Provisional application No. 61/044,773, filed on Apr. 14, 2008.

(51) Int. Cl. A61K 31/65; A61P 31/04

(52) U.S. Cl. 514/152

(57) ABSTRACT

The present invention pertains, at least in part, to methods of treating a microorganism-associated infection in a subject comprising administering to said subject an effective amount of a tetracycline compound.

### RELATED APPLICATIONS

This application claims priority to U.S. Provisional Patent Application No. 61/044,773 filed on Apr. 14, 2008. The contents of the aforementioned application are hereby incorporated by reference in their entirety.

### BACKGROUND

The development of the tetracycline antibiotics was the direct result of a systematic screening of soil specimens collected from many parts of the world for evidence of microorganisms capable of producing bactericidal and/or bacteriostatic compositions. The first of these novel compounds was introduced in 1948 under the name chlortetracycline. Two years later, oxytetracycline became available. The elucidation of the chemical structure of these compounds confirmed their similarity and furnished the analytical basis for the production of a third member of this group in 1952, tetracycline. A new family of tetracycline compounds, without the ring-attached methyl group present in earlier tetracyclines, was prepared in 1957 and became publicly available in 1967; and minocycline was in use by 1972.

Recently, research efforts have focused on developing new tetracycline antibiotic compositions effective under varying therapeutic conditions and routes of administration. New tetracycline analogues have also been investigated which may prove to be equal to or more effective than the originally introduced tetracycline compounds. Examples include U.S. Pat. Nos. 2,980,584; 2,990,331; 3,062,717; 3,165,531; 3,454,697; 3,557,280; 3,674,859; 3,957,980; 4,018,889; 4,024,272; and 4,126,680. These patents are representative of the range of pharmaceutically active tetracycline and tetracycline analogue compositions.

Historically, soon after their initial development and introduction, the tetracyclines were found to be highly effective pharmacologically against rickettsiae; a number of gram-positive and gram-negative bacteria; and the agents responsible for lymphogranuloma venereum, inclusion conjunctivitis, and psittacosis. Hence, tetracyclines became known as “broad spectrum” antibiotics. With the subsequent establishment of their in vitro antimicrobial activity, effectiveness in experimental infections, and pharmacological properties, the tetracyclines as a class rapidly became widely used for therapeutic purposes. However, this widespread use of tetracyclines for both major and minor illnesses and diseases led directly to the emergence of resistance to these antibiotics even among highly susceptible bacterial species both commensal and pathogenic (e.g., pneumococci and Salmonella). The rise of tetracycline-resistant organisms has resulted in a general decline in use of tetracyclines as antibiotics of choice.

### SUMMARY OF THE INVENTION

In one embodiment, the invention pertains, at least in part, to methods of treating a microorganism-associated infection in a subject comprising administering to said subject an effective amount of a tetracycline compound of Formula I:

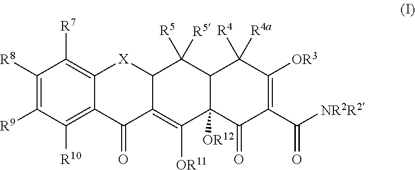

wherein

X is CHC(R¹³Y′Y), CR^{6′}R⁶, C═CR^{6′}R⁶, S, NR⁶, or O;

R², R^{2′}, R^{4′}, and R^{4″} are each independently hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or a prodrug moiety;

R³, R^{4a}, R¹¹ and R¹² are each independently hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or a prodrug moiety;

R⁴ is NR^{4′} R^{4″}, hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or a prodrug moiety;

R⁵ and R^{5′} are each hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or a prodrug moiety;

R⁶ and R^{6′} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;

R⁷ is hydrogen, alkyl, alkenyl, alkynyl, acyl, hydroxyl, alkoxy, halogen, thioether, sulfinyl, sulfonyl, amino, cyano, nitro, carbonyl, oximyl, aryl, heterocyclic or —(CH₂)₀₋₃(NR^{7c})₀₋₁C(═W′)WR^{7a};

R⁸ is hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or —(CH₂)₀₋₃(NR^{8c})₀₋₁C(=E′)ER^{8a};

R⁹ is hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or —(CH₂)₀₋₃(NR^{9c})₀₋₁C(=Z′)ZR^{9a};

R¹⁰ is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;

R^{7a}, R^{7b}, R^{7c}, R^{7d}, R^{7e}, R^{7f}, R^{8a}, R^{8b}, R^{8c}, R^{8d}, R^{8e}, R^{8f}, R^{9a}, R^{9b}, R^{9c}, R^{9d}, R^{9e}, and R^{9f} are each hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;

R¹³ is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;

E is CR^{8d}R^{8e}, S, NR^{8b} or O;

E′ is O, NR^{8f}, or S;

W is CR^{7d}R^{7e}, S, NR^{7b} or O;

W′ is O, NR^{7f}, or S;

X is CHC(R¹³Y′Y), C═CR¹³Y, CR^{6′}R⁶, S, NR⁶, or O;

Z is CR^{9d}R^{9e}, S, NR^{9b} or O;

Z′ is O, S, or NR^{9f};

Y′ and Y are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; or a pharmaceutically acceptable salt, ester or enantiomer thereof.

In another embodiment, the invention pertains, at least in part, to methods of treating a microorganism-associated infection in a subject comprising administering to said subject an effective amount of a tetracycline compound of formula II:

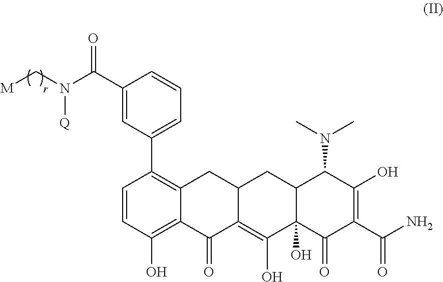

wherein

r is an integer from 1 to 10;

M is OR^{7o}* or NR^{7p}*R^{7q}*;

Q is hydrogen or alkyl;

R^{7o}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7p}* and R^{7q}* are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7p}* and R^{7q}* are linked to form a 5- or 6-membered aryl, heterocyclic or aliphatic ring;

or a pharmaceutically acceptable salt, ester or enantiomer thereof.

In yet another embodiment, the invention pertains, at least in part, to methods of treating a microorganism-associated infection in a subject comprising administering to said subject an effective amount of a tetracycline compound of formula III:

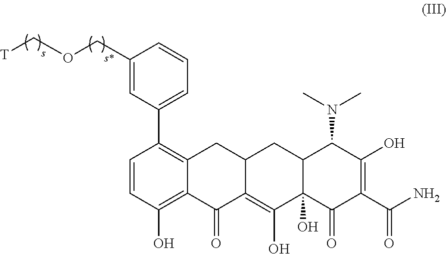

wherein

s and s* are each independently an integer from 1 to 10;

T is OR^{7r}* or NR^{7s}*R^{7t}*;

R^{7r}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7s}* and R^{7t}* are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7s}* and R^{7t}* are linked to form a 5- or 6-membered aryl, heterocyclic or aliphatic ring;

or a pharmaceutically acceptable salt, ester or enantiomer thereof.

In one embodiment, the invention, pertains, at least in part, to methods of treating a microorganism-associated infection in a subject comprising administering to said subject an effective amount of a tetracycline compound of formula IV:

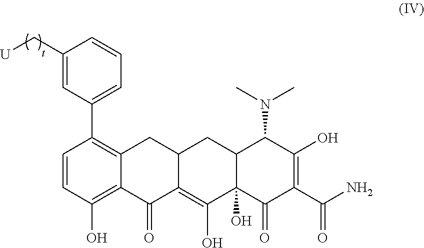

wherein

t is an integer from 1 to 10;

U is OR^{7u}* or NR^{7v}*R^{7w}*;

R^{7u}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7v}* and R^{7w}* are each hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7v}* and R^{7w}* are linked to form a 5- or 6-membered aryl, heterocyclic or aliphatic ring;

or a pharmaceutically acceptable salt, ester or enantiomer thereof.

In yet another embodiment, the invention pertains, at least in part, to methods of treating a microorganism-associated infection in a subject comprising administering to said subject an effective amount of a tetracycline compound of formula V:

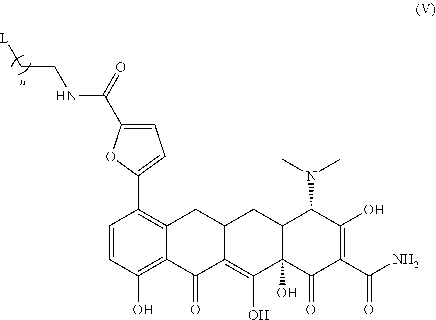

wherein

u is an integer from 1 to 10;

L is OR^{7x}* or NR^{7y}*R^{7z}*;

R^{7x}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7y}* and R^{7z}* are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7y}* and R^{7z}* are linked to form a 5- or 6-membered aryl, heterocyclic or aliphatic ring;

or a pharmaceutically acceptable salt, ester or enantiomer thereof.

In another embodiment, the invention pertains, at least in part, to methods of treating a microorganism-associated infection in a subject comprising administering to said subject an effective amount of a tetracycline compound of formula VI:

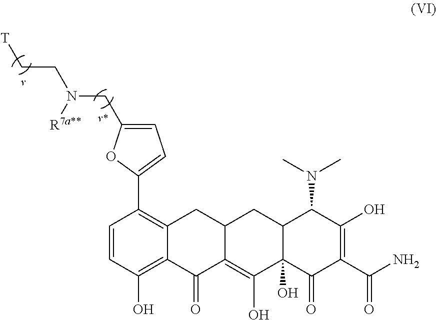

wherein

v and v* are each independently an integer from 1 to 10;

T is OR^{7b}** or NR^{7c}**R^{7d}**;

R^{7b}** is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7c}** and R^{7d}** are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7c}** and R^{7d}** are linked to form a 5- or 6-membered aryl, heterocyclic or aliphatic ring;

or a pharmaceutically acceptable salt, ester or enantiomer thereof.

In another embodiment, the invention pertains, at least in part, to methods of treating a microorganism-associated infection in a subject comprising administering to said subject an effective amount of a tetracycline compound of formula VII:

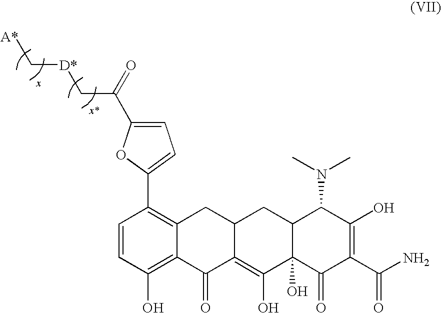

wherein

x and x* are each independently an integer from 1 to 10;

A* is OR^{7e}** or NR^{7f}**R^{7g}**;

D* is NH, NCH₃, O, CH₂;

R^{7e}** is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7f}** and R^{7g}** are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; or R^{7f}** and R^{7g}** are linked to form a 5- or 6-membered aryl, heterocyclic or aliphatic ring;

or a pharmaceutically acceptable salt, ester or enantiomer thereof.

In a further embodiment, the invention pertains, at least in part, to methods of treating a microorganism-associated infection in a subject comprising administering to said subject an effective amount of a tetracycline compound of formula VIII:

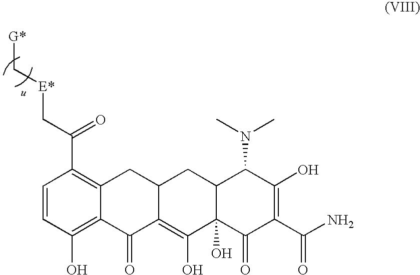

wherein

u is an integer from 1 to 10;

G* is OR^{7h}** or NR⁷ⁱ**R^{7j}**;

E* is NH, NCH₃, O, CH₂;

R^{7h}** is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R⁷ⁱ** and R^{7j}** are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R⁷ⁱ** and R^{7j}** are linked to form a 5- or 6-membered aryl, heterocyclic or aliphatic ring;

or a pharmaceutically acceptable salt, ester or enantiomer thereof.

In yet another embodiment, the invention pertains, at least in part, to methods of treating a microorganism-associated infection in a subject comprising administering to said subject an effective amount of a tetracycline compound of formula IX:

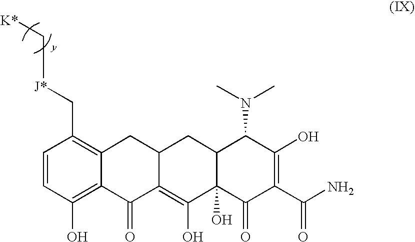

wherein

y is an integer from 1 to 10;

K* is OR^{7k}** or NR^{7l**}R^{7m}**;

J* is NH, NCH₃, O, CH₂;

R^{7k}** is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7l}** and R^{7m}** are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7l}** and R^{7m}** are linked to form a 5- or 6-membered aryl, heterocyclic or aliphatic ring;

or a pharmaceutically acceptable salt, ester or enantiomer thereof.

In another embodiment, the invention pertains, at least in part, to methods of treating a microorganism-associated infection in a subject comprising administering to said subject an effective amount of a tetracycline compound of formula X:

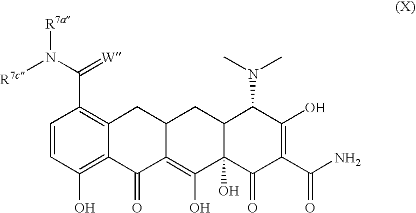

wherein W″ is CR^{7d″}R^{7e″}, S, NR^{7b″} or O; and

R^{7a″}, R^{7b″}, R^{7c″}, R^{7d″} and R^{7e″} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7a}″ and R^{7c″} are linked together to form a 5- or 6-membered aryl, heterocyclic or aliphatic ring; or a pharmaceutically acceptable salt, ester or enantiomer thereof.

In one embodiment, the invention pertains, at least in part, to a pharmaceutical composition for the treatment of a microorganism-associated infection comprising a therapeutically effective amount of a tetracycline compound of the invention, e.g., a compound of Formula I, II, III, IV, V, VI, VII, VIII, IX or X or a compound listed in Table 2, and a pharmaceutically acceptable carrier.

In another further embodiment, the invention pertains, at least in part, to methods for treating a subject for a microorganism-associated infection by administering an effective amount of a tetracycline compound of the invention, e.g., a compound of Formula I, II, III, IV, V, VI, VII, VIII, IX or X or a compound listed in Table 2 or a tetracycline compound otherwise described herein.

In another further embodiment, the invention pertains, at least in part, to the use of a tetracycline compound in the manufacture of a medicament for treating a microorganism-associated infection, wherein said medicament comprises an effective amount of a tetracycline compound of the invention, e.g., a compound of Formula I, II, III, IV, V, VI, VII, VIII, IX or X or a compound listed in Table 2 or a salt, ester or enantiomer thereof.

### DETAILED DESCRIPTION OF THE INVENTION

The present invention pertains, at least in part, to use of a substituted tetracycline compound, for example, to treat a microorganism-associated infection (e.g., a bacterial infection). The term “tetracycline compound” includes many compounds with a similar ring structure to tetracycline. Examples of tetracycline compounds include: chlortetracycline, oxytetracycline, demeclocycline, methacycline, sancycline, chelocardin, rolitetracycline, lymecycline, apicycline; clomocycline, guamecycline, meglucycline, mepylcycline, penimepicycline, pipacycline, etamocycline, penimocycline, etc. Other derivatives and analogues comprising a similar four ring structure are also included (See Rogalski, “Chemical Modifications of Tetracyclines,” the entire contents of which are hereby incorporated herein by reference). Table 1 depicts tetracycline and several known other tetracycline derivatives.

TABLE 1

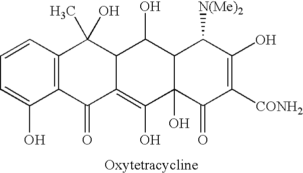

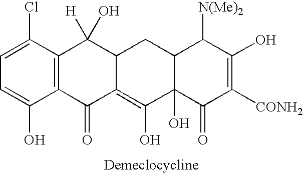

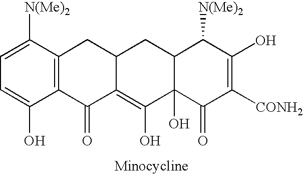

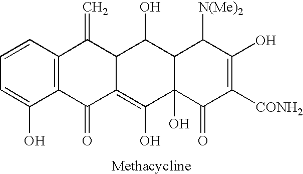

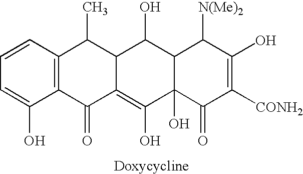

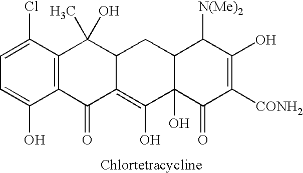

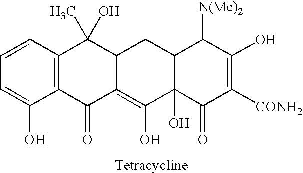

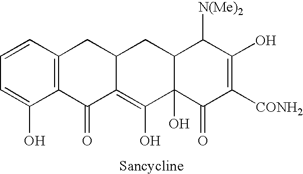

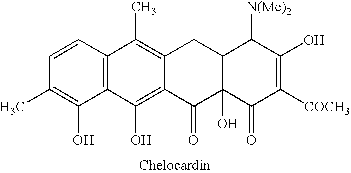

Other tetracycline compounds which may be modified using the methods of the invention include, but are not limited to, 6-demethyl-6-deoxy-4-dedimethylaminotetracycline; tetracyclino-pyrazole; 7-chloro-4-dedimethylaminotetracycline; 4-hydroxy-4-dedimethylaminotetracycline; 12α-deoxy-4-dedimethylaminotetracycline; 5-hydroxy-6α-deoxy-4-dedimethylaminotetracycline; 4-dedimethylamino-12α-deoxyanhydrotetracycline; 7-dimethylamino-6-demethyl-6-deoxy-4-dedimethylaminotetracycline; tetracyclinonitrile; 4-oxo-4-dedimethylaminotetracycline 4,6-hemiketal; 4-oxo-11a C1-4-dedimethylaminotetracycline-4,6-hemiketal; 5a,6-anhydro-4-hydrazon-4-dedimethylamino tetracycline; 4-hydroxyimino-4-dedimethylamino tetracyclines; 4-hydroxyimino-4-dedimethylamino 5a,6-anhydrotetracyclines; 4-amino-4-dedimethylamino-5a,6 anhydrotetracycline; 4-methylamino-4-dedimethylamino tetracycline; 4-hydrazono-11a-chloro-6-deoxy-6-demethyl-6-methylene-4-dedimethylamino tetracycline; tetracycline quaternary ammonium compounds; anhydrotetracycline betaines; 4-hydroxy-6-methyl pretetramides; 4-keto tetracyclines; 5-keto tetracyclines; 5a,11a dehydro tetracyclines; 11a C1-6, 12 hemiketal tetracyclines; 11a C1-6-methylene tetracyclines; 6,13 diol tetracyclines; 6-benzylthiomethylene tetracyclines; 7,11a-dichloro-6-fluoro-methyl-6-deoxy tetracyclines; 6-fluoro (α)-6-demethyl-6-deoxy tetracyclines; 6-fluoro (β)-6-demethyl-6-deoxy tetracyclines; 6-αacetoxy-6-demethyl tetracyclines; 6-β acetoxy-6-demethyl tetracyclines; 7,13-epithiotetracyclines; oxytetracyclines; pyrazolotetracyclines; 11a halogens of tetracyclines; 12a formyl and other esters of tetracyclines; 5, 12a esters of tetracyclines; 10,12a-diesters of tetracyclines; isotetracycline; 12-a-deoxyanhydro tetracyclines; 6-demethyl-12a-deoxy-7-chloroanhydrotetracyclines; B-nortetracyclines; 7-methoxy-6-demethyl-6-deoxytetracyclines; 6-demethyl-6-deoxy-5a-epitetracyclines; 8-hydroxy-6-demethyl-6-deoxy tetracyclines; monardene; chromocycline; 5a methyl-6-demethyl-6-deoxy tetracyclines; 6-oxa tetracyclines, and 6 thia tetracyclines.

For example, the tetracycline compound used in the methods of the invention is not a compound shown in Table 1 (for example, oxytetracycline (e.g., a compound of formula I in which X is CR⁶R⁶; R²R^{2′}R³R^{4a}, R^{5′} R⁷, R⁸, R⁹, R¹¹ and R¹² are hydrogen; R⁵ and R¹⁰ are hydroxyl; R^{6′} is methyl; R⁴ is NR^{4′}R^{4″} and R^{4′} and R^{4″} are methyl), demeclocycline (e.g., a compound of formula I in which X is CR⁶R^{6′}; R², R^{2′}, R³, R^{4a}, R⁵, R^{5′}, R^{6′}, R⁸, R⁹, R¹¹, R¹² are hydrogen; R^{6′} and R¹⁰ are each hydroxyl; R⁷ is chlorine; R⁴ is NR^{4′}R^{4″} and R^{4′} and R^{4″} are methyl), minocycline (e.g., a compound of formula I in which X is CR⁶R^{6′}; R², R^{2′}, R³,
R^{4a}, R⁵, R^{5′}, R⁶, R^{6′}, R⁸, R⁹, R¹¹ and R¹² are hydrogen, R⁷ is —N(CH₃)₂, R¹⁰ is hydroxyl; R⁴ is NR^{4′}R^{4″} and R^{4′} and R^{4″} are methyl), methacycline (e.g., a compound of formula I in which X is C=6R⁶R^{6′}; R²R^{2′}, R³, R^{4a}, R⁵, R⁶, R^{6′}, R⁷, R⁸, R⁹, R¹¹ and R¹² are hydrogen; R^{5′} and R¹⁰ are hydroxyl; R⁴ is NR^{4′}R^{4″} and R^{4′} and R^{4″} are methyl), doxycycline, (e.g., a compound of formula I in which X is CR⁶R^{6′}; R², R^{2′}, R³, R^{4a}, R⁵, R⁶, R^{6′}, R⁷, R⁸, R⁹, R¹¹ and R¹² are hydrogen; R^{5′} and R¹⁰ are hydroxyl; R^{6′} is methyl; R⁴ is NR^{4′}R^{4″} and R^{4′} and R^{4″} are methyl), chlortetracycline (e.g., a compound of formula I in which X is CR⁶R^{6′}; R², R^{2′}, R³,
R^{4a}, R⁵, R^{5′}, R⁸, R⁹, R¹¹ and R¹² are hydrogen; R⁶ and R¹⁰ are hydroxyl; R⁶ is methyl; R⁷ is chlorine; R⁴ is NR^{4′}R^{4″} and R^{4′} and R^{4″} are methyl), tetracycyline (e.g., a compound of formula I in which X is CR⁶R^{6′}; R², R^{2′}, R³, R^{4a}, R⁵, R^{5′}, R⁷, R⁸, R⁹, R¹¹, R¹² are hydrogen;
R⁶ and R¹⁰ are hydroxyl; R^{6′} is methyl; R⁴ is NR^{4′}R^{4″} and R^{4′} and R^{4″} are methyl) or sancycline (e.g., a compound of formula I in which X is CR⁶R^{6′}; R², R^{2′}, R^{4a}, R⁵, R^{5′}, R⁶, R^{6′}, R⁷, R⁸, R⁹, R¹¹ and R¹² are hydrogen; R¹⁰ is hydroxyl; R⁴ is NR^{4′}R^{4″} and R^{4′} and R^{4″} are methyl)).

The term “tetracycline compound” also includes tetracycline compounds with one or more additional substituents, e.g., at the 1, 2, 3, 4, 5, 6, 7, 8, 9, 10, 11, 11a, 12, 12a or 13 position or at any other position which allows the substituted tetracycline compound of the invention to perform its intended function, e.g., treat spinal muscular atrophy. In one embodiment, the tetracycline compound is a substituted oxytetracycline compound (e.g., R⁴ is NR^{4′}R^{4″}, R^{4a} and R^{5′} are each hydrogen, R⁵ is hydroxyl, X is CR⁶R^{6′}, R⁶ is hydroxyl and R^{6′} is methyl). In another embodiment, the tetracycline compound is a substituted minocycline compound (e.g., R⁴ is NR^{4′}R^{4″}, X is CR⁶R^{6′}, R^{4a}, R⁵, R^{5′}, R⁶ and R^{6′} are each hydrogen and R⁷ is N(CH₃)₂). In yet another embodiment, the tetracycline compound is a substituted doxycycline compound (e.g., R⁴ is NR^{4′}R^{4″}, X is CR⁶R⁶, R^{4a} and R^{5′} are each hydrogen, R⁵ is hydroxyl, R⁶ is methyl and R^{6′} is hydrogen). In another embodiment, the tetracycline compound is a substituted tetracycline compound (e.g., R⁴ is NR^{4′}R^{4″}, X is CR⁶R^{6′}, R^{4a}, R⁵ and R^{5′} are each hydrogen, R⁶ is methyl and R^{6′} is hydroxyl). In one embodiment, the tetracycline compound is a substituted sancycline compound (e.g., R⁴ is NR^{4′}R^{4″}, X is CR⁶R^{6′}, R^{4a}, R^{5′}, R⁵, R⁶ and R^{6′} are each hydrogen). In another embodiment, the tetracycline compound is a substituted demeclocycline compound (e.g., R⁴ is NR^{4′}R^{4″}, X is CR⁶R^{6′}, R^{4a}, R⁵, R^{5′} and R⁶ are hydrogen, R^{6′} is hydroxyl and R⁷ is chlorine). In another embodiment, the tetracycline compound is a substituted methacycline compound (e.g., R⁴ is NR^{4′}R^{4″}, X is C═CR^{6′}R⁶, R⁵ is hydroxyl and R^{4a}, R^{5′}, R^{6′} and R⁶ are hydrogen). In another embodiment, the tetracycline compound is a substituted chlortetracycline compound (e.g., R⁴ is NR^{4′}R⁴, X is CR⁶R^{6′}, R^{4a} and R^{5′} are hydrogen, R⁵ is hydroxyl, and R⁶ is methyl, R^{6′} is hydroxyl and R⁷ is chlorine). In certain embodiments, the substituted tetracycline compound is a 7-substituted sancycline compound, a 9-substituted minocycline compound, or a 7,9-substituted sancycline compound.

A “tetracycline compound” used in methods of the invention includes compounds of the formula (I):

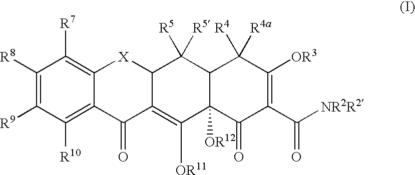

wherein

X is CHC(R¹³Y′Y), CR^{6′}R⁶, C═CR^{6′}, R⁶, S, NR⁶, or O;

R², R^{2′}, R^{4′}, and R⁴″ are each independently hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or a prodrug moiety;

R³, R^{4a}, R¹¹ and R¹² are each independently hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or a prodrug moiety;

R⁴ is NR^{4′}R^{4″}, hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or a prodrug moiety;

R⁵ and R^{5′} are each hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or a prodrug moiety;

R⁶ and R^{6′} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;

R⁷ is hydrogen, alkyl, alkenyl, alkynyl, acyl, hydroxyl, alkoxy, halogen, thioether, sulfinyl, sulfonyl, amino, cyano, nitro, carbonyl, oximyl, aryl, heterocyclic or —(CH₂)₀₋₃ (NR^{7c})₀₋₁C(═W′)WR^{7a};

R⁸ is hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or —(CH₂)₀₋₃(NR^{8c})₀₋₁C(=E′)ER^{8a};

R⁹ is hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or —(CH₂)₀₋₃(NR^{9c})₀₋₁C(=Z′)ZR^{9a};

R¹⁰ is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;

R^{7a}, R^{7b}, R^{7c}, R^{7d}, R^{7e}, R^{7f}, R^{8a}, R^{8b}, R^{8c}, R^{8d}, R^{8e}, R^{8f}, R^{9a}, R^{9b}, R^{9c}, R^{9d}, R^{9e}, and R^{9f} are each hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;

R¹³ is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;

E is CR^{8d}R^{8e}, S, NR^{8b} or O;

E′ is O, NR^{8f}, or S;

W is CR^{7d}R^{7e}, S, NR^{7b} or O;

W′ is O, NR^{7f}, or S;

X is CHC(R¹³Y′Y), C═CR¹³Y, CR^{6′}R⁶, S, NR⁶, or O;

Z is CR^{9d}R^{9e}, S, NR^{9b} or O;

Z′ is O, S, or NR^{9f};

Y′ and Y are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; or a pharmaceutically acceptable salt, ester or enantiomer thereof.

In one embodiment, X is CR⁶R^{6′}; R^{2′}, R^{2″}, R³, R^{4a}, R⁵, R^{5′}, R⁶, R^{6′}, R⁸, R⁹, R¹¹ and R¹² are each hydrogen; R⁴ is NR^{4′}R^{4″} and R^{4′} and R^{4″} are each alkyl (e.g., methyl) and R⁷ is aryl, for example, of formula XI:

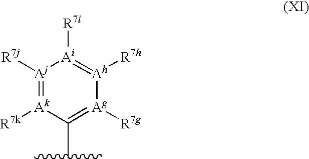

wherein

A^{g}, A^{h}, Aⁱ, A^{j} and A^{k} are each independently N or C; and

when A^{g}, A^{h}, Aⁱ, A^{j} and A^{k} are C; R^{7g}, R^{7h}, R⁷ⁱ, R^{7j} and R^{7k} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7j} and R⁷ⁱ are linked to form a 5- or 6-membered aryl, heterocyclic or aliphatic ring; or

R^{7g}, R^{7h}, R⁷ⁱ, R^{7j} and R^{7k} are absent when A^{g}, A^{h}, Aⁱ, A^{j} and A^{k} are N.

In another embodiment, A^{g}, A^{h}, Aⁱ, A^{j} or A^{k} are each C; R^{7h}, R⁷ⁱ and R^{7k} are each hydrogen and R^{7j} is carbonyl, for example, of formula XII:

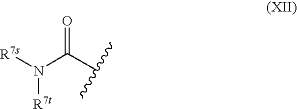

wherein

R^{7s} and R^{7t} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7s} and R^{7t} are linked to form a 5- or 6-membered aryl, heterocyclic or aliphatic ring.

In one embodiment, R^{7t} is hydrogen and R^{7s} is alkyl, for example, formula XIII:

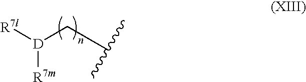

wherein

D is O, N, NR^{7′} or CR^{7′};

n is an integer from 0 to 10;

R^{7′} is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

when D is N or CR^{7′}, R^{7l} and R^{7m} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7l} and R^{7m} are linked to form a 5- or 6-membered aryl, heterocyclic or aliphatic ring; and

when D is O, R^{7l} is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic and R^{7m} is absent.

In another embodiment, D is N; n is 2 and R^{7l} and R^{7m} are linked to form a 5-membered heterocyclic ring (e.g., pyrrolyl). Alternatively, D is NR^{7′}; n is 2 and R^{7′}, R^{7l} and R^{7m} are each alkyl (e.g., methyl).

In yet another embodiment, R^{7j} is alkyl, for example, of formula XIII:

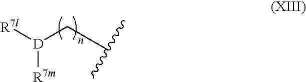

wherein

D is O, N, NR^{7′} or CR^{7′};

n is an integer from 0 to 10;

R^{7′} is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

when D is N or CR^{7′}, R^{7l} and R^{7m} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7l} and R^{7m} are linked to form a 5- or 6-membered aryl, heterocyclic or aliphatic ring; and

when D is O, R^{7l} is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic and R^{7m} is absent.

In one embodiment, n is 1; D is N; R^{7m} is hydrogen or alkyl (e.g., methyl) and R^{7l} is alkyl, for example, of formula XIV:

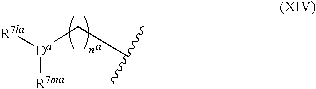

wherein

D^{a} is O, N, NR^{7a′} or CR^{7a′};

n^{a} is an integer from 0 to 10;

R^{7a′} is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

when D^{a} is N or CR^{7a′}, R^{7la} and R^{7ma} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7la} and R^{7ma} are linked to form a 5- or 6-membered aryl, heterocyclic or aliphatic ring; and

when D^{a} is O, R^{7la} is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic and R^{7ma} is absent.

In another embodiment, D^{a} is N; R^{7la} and R^{7ma} are each alkyl and n^{a} is 2, 3 or 4.

In one embodiment, R⁷ is aryl, for example, of formula XV:

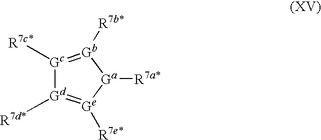

wherein

G^{a} is N, O, S or CR^{7f}*;

G^{b}, G^{c}, G^{d} and G^{e} are each independently N or CR^{7f}*;

R^{7f}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;

R^{7a}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic when G^{a} is N or CR^{7f}* or R^{7a}* is absent when G^{a} is O or S;

R^{7b}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic when G^{b} is CR^{7f}* or R^{7b}* is absent when G^{b} is N;

R^{7c}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic when G^{c} is CR^{7f}* or R^{7c}* is absent when G^{c} is N;

R^{7d}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7d}* is covalently bonded to the 7-position of the tetracycline compound when G^{d} is CR^{7f}*; or R^{7d}* is absent when G^{d} is N; and

R^{7e}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7e}* is covalently bonded to the 7-position of the tetracycline compound when G^{e} is CR^{7f}* or R^{7e}* is absent when G^{e} is N;

provided that one of R^{7d}* or R^{7e}* are covalently bonded to the 7-position of the tetracycline compound.

In one embodiment, R^{7e}* is covalently bonded to the 7-position of the tetracycline compound; G^{a} is O; R^{7c}* and R^{7d}* are each hydrogen and R^{7b}* is alkyl, for example, of formula XIII:

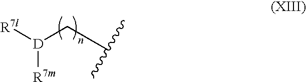

wherein

D is O, N, NR^{7′} or CR^{7′};

n is an integer from 0 to 10;

R^{7′} is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

when D is N or CR^{7′}, R^{7l} and R^{7m} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7l} and R^{7m} are linked to form a 5- or 6-membered aryl, heterocyclic or aliphatic ring; and

when D is O, R^{7l} is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic and R^{7m} is absent.

In another embodiment, R^{7m} is hydrogen or alkyl (e.g., methyl); R^{7l} is alkyl, for example, of formula XIV:

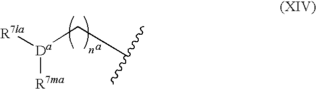

wherein

D^{a} is O, N, NR^{7a′} or CR^{7a′};

n^{a} is an integer from 0 to 10;

R^{7a′} is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

when D^{a} is N or CR^{7a′}, R^{7la} and R^{7ma} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7la} and R^{7ma} are linked to form a 5- or 6-membered aryl, heterocyclic or aliphatic ring; and

when D^{a} is O, R^{7la} is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic and R^{7ma} is absent.

In a further embodiment, n^{a} is 2; D^{a} is N and R^{7la} and R^{7ma} are each alkyl (e.g., methyl).

In another embodiment, the tetracycline compound used in methods of the invention includes compounds of formula II:

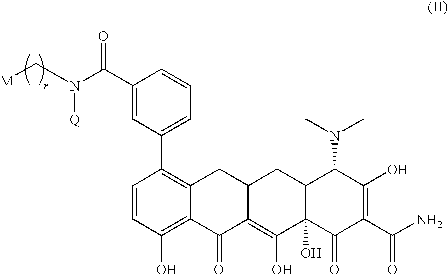

wherein

r is an integer from 1 to 10;

M is OR^{7o}* or NR^{7p}*R^{7q}*;

Q is hydrogen or alkyl;

R^{7o}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7p}* and R^{7q}* are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7p}* and R^{7q}* are linked to form a 5- or 6-membered aryl, heterocyclic or aliphatic ring;

or a pharmaceutically acceptable salt, ester or enantiomer thereof.

In yet another embodiment, the tetracycline compound used in methods of the invention includes compounds of formula III:

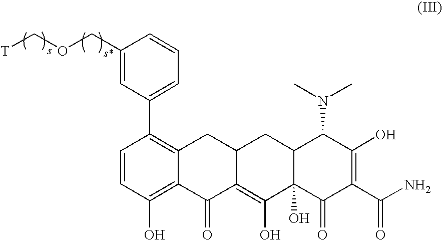

wherein

s and s* are each independently an integer from 1 to 10;

T is OR^{7r}* or NR^{7s}*R^{7t}*;

R^{7r}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7s}* and R^{7t}* are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7s}* and R^{7t}* are linked to form a 5- or 6-membered aryl, heterocyclic or aliphatic ring;

or a pharmaceutically acceptable salt, ester or enantiomer thereof.

In one embodiment, the tetracycline compound used in methods of the invention includes compounds of formula IV:

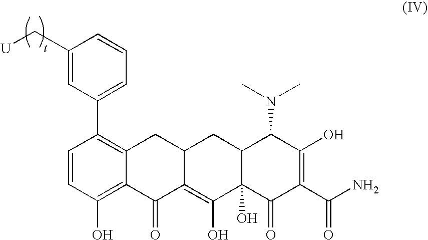

wherein

t is an integer from 1 to 10;

U is OR^{7u}* or NR^{7v}*R^{7w}*;

R^{7u}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7v}* and R^{7w}* are each hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7v}* and R^{7w}* are linked to form a 5- or 6-membered aryl, heterocyclic or aliphatic ring;

or a pharmaceutically acceptable salt, ester or enantiomer thereof.

In another embodiment, the tetracycline compound used in methods of the invention includes compounds of formula V:

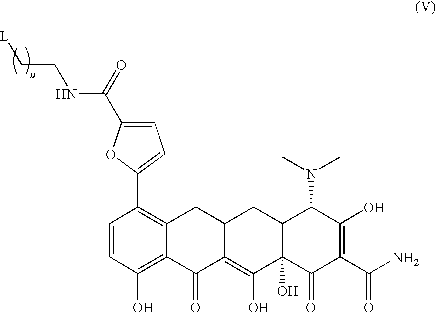

wherein

u is an integer from 1 to 10;

L is OR^{7x}* or NR^{7y}*R^{7z}*;

R^{7x}* is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7y}* and R^{7z}* are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7y}* and R^{7z}* are linked to form a 5- or 6-membered aryl, heterocyclic or aliphatic ring;

or a pharmaceutically acceptable salt, ester or enantiomer thereof.

In one embodiment, the tetracycline compound used in methods of the invention includes compounds of formula VI:

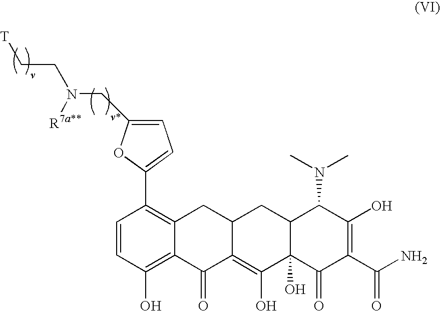

wherein

v and v* are each independently an integer from 1 to 10;

T is OR^{7b}** or NR^{7c}**R^{7d}**;

R^{7a}** and R^{7b}** are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7c}** and R^{7d}** are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7c}** and R^{7d}** are linked to form a 5- or 6-membered aryl, heterocyclic or aliphatic ring;

or a pharmaceutically acceptable salt, ester or enantiomer thereof.

In another embodiment, the tetracycline compound used in methods of the invention includes compounds of formula VII:

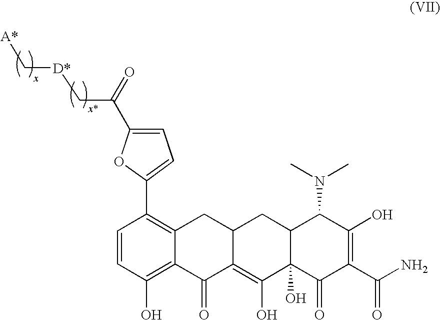

wherein

x and x* are each independently an integer from 1 to 10;

A* is OR^{7e}** or NR^{7f}**R^{7g}**;

D* is NH, NCH₃, O, CH₂;

R^{7e}** is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7f}** and R^{7g}** are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; or R^{7f}** and R^{7g}** are linked to form a 5- or 6-membered aryl, heterocyclic or aliphatic ring;

or a pharmaceutically acceptable salt, ester or enantiomer thereof.

In a further embodiment, the tetracycline compound used in methods of the invention includes compounds of formula VIII:

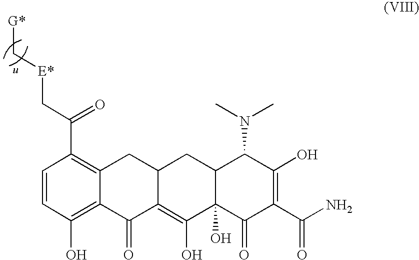

wherein

u is an integer from 1 to 10;

G* is OR^{7h}** or NR⁷ⁱ** R^{7j}**;

E* is NH, NCH₃, O, CH₂;

R^{7h}** is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R⁷ⁱ** and R^{7j}** are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R⁷ⁱ** and R^{7j}** are linked to form a 5- or 6-membered aryl, heterocyclic or aliphatic ring;

or a pharmaceutically acceptable salt, ester or enantiomer thereof.

In yet another embodiment, the tetracycline compound used in methods of the invention includes compounds of formula IX:

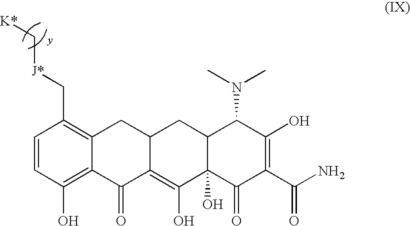

wherein

y is an integer from 1 to 10;

K* is OR^{7k}** or NR^{7l}**R^{7m}**

J* is NH, NCH₃, O, CH₂;

R^{7k}** is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and

R^{7l}* and R^{7m}* are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7l}** and R^{7m}** are linked to form a 5- or 6-membered aryl, heterocyclic or aliphatic ring;

or a pharmaceutically acceptable salt, ester or enantiomer thereof.

In another embodiment, the tetracycline compound used in methods of the invention includes compounds of formula X:

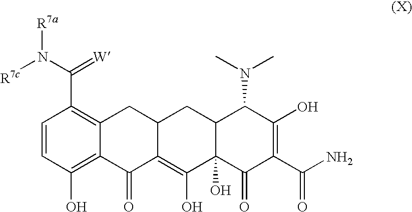

wherein W′ is CR^{7d}R^{7e}, S, NR^{7b} or O; and

R^{7a}, R^{7b}, R^{7c}, R^{7d} and R^{7e} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7a} and R^{7b} are linked together to form a 5- or 6-membered aryl, heterocyclic or aliphatic ring; or a pharmaceutically acceptable salt, ester or enantiomer thereof.

Examples of tetracycline compounds used in the methods of the invention include compounds of Table 2, and pharmaceutically acceptable salts, esters and enantiomers thereof.

TABLE 2

| | |
|---|---|
| A | 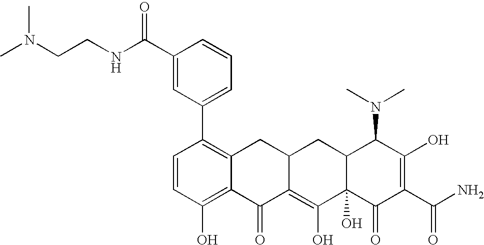 |
| B | 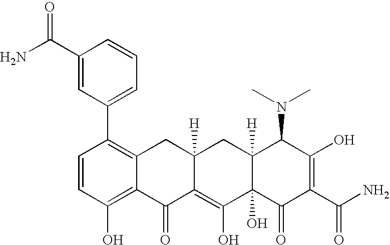 |
| C | 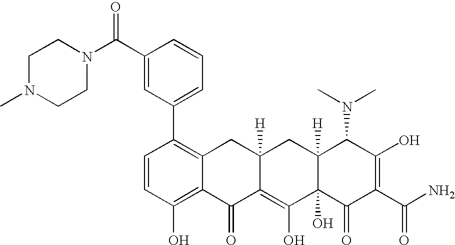 |
| D | 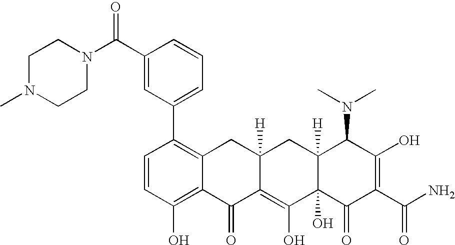 |
| E | 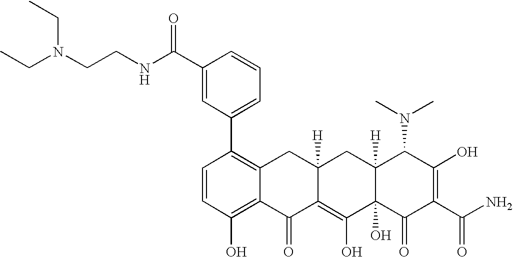 |
| F | 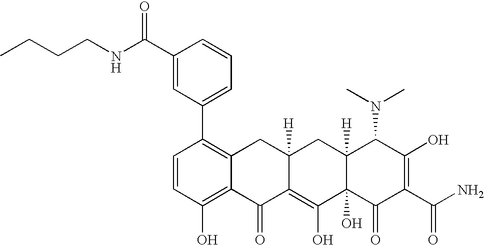 |
| G | 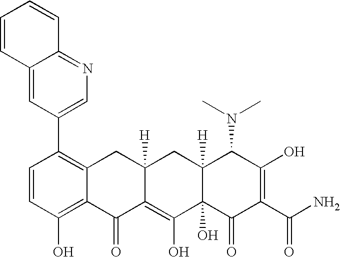 |
| H | 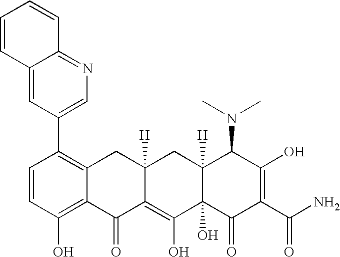 |
| I | 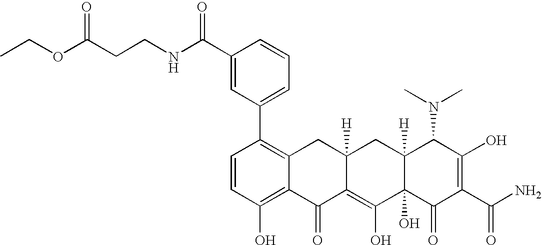 |
| J | 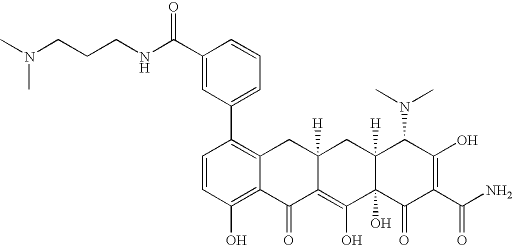 |
| K | 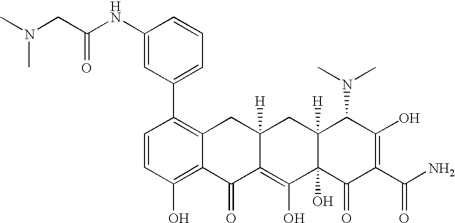 |
| L | 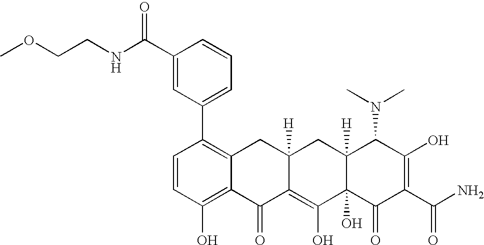 |
| M | 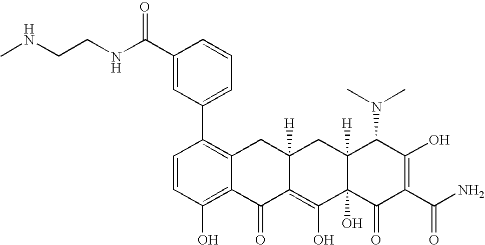 |
| N | 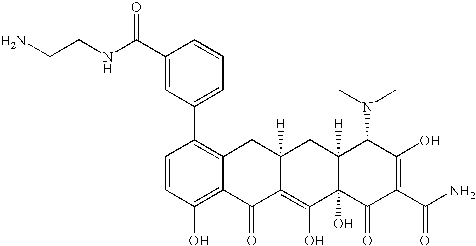 |
| O | 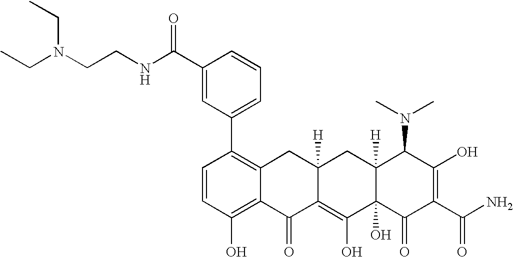 |
| P | 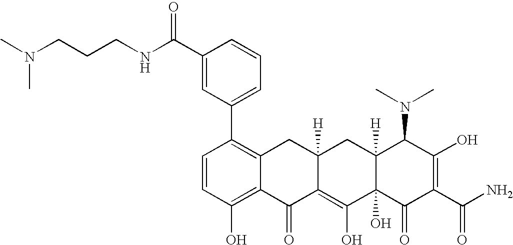 |
| Q | 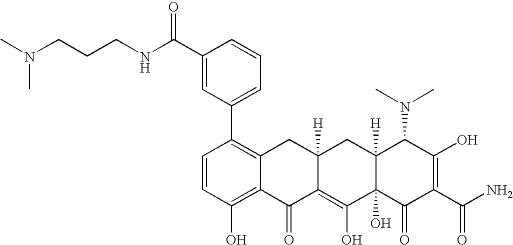 |
| R | 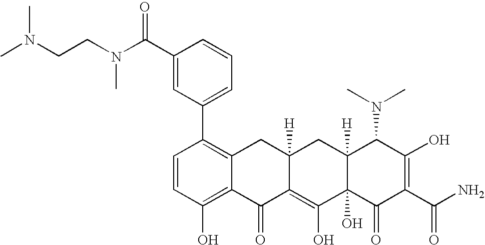 |
| S | 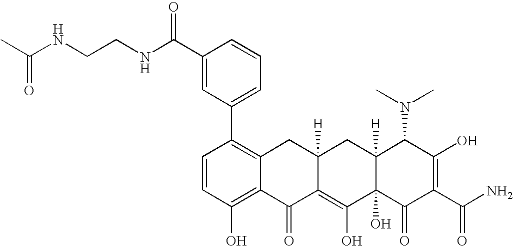 |
| T | 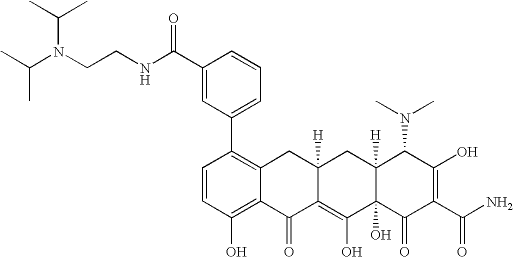 |
| U | 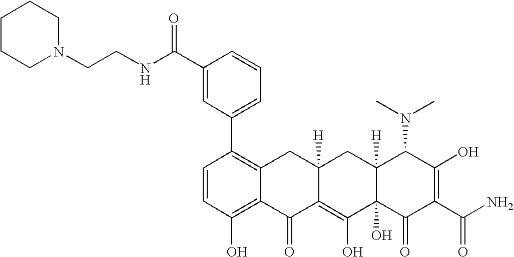 |
| V | 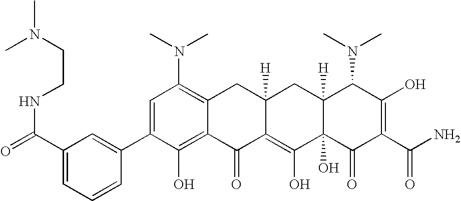 |
| W | 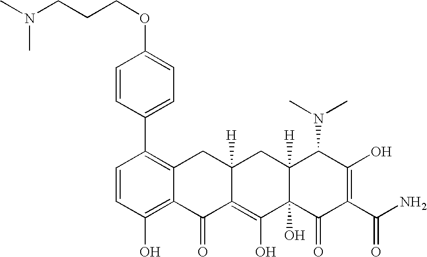 |
| X | 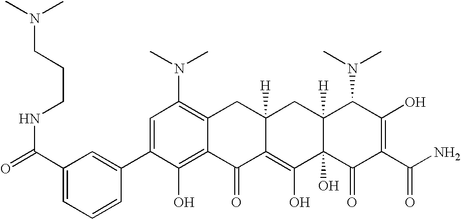 |
| Y | 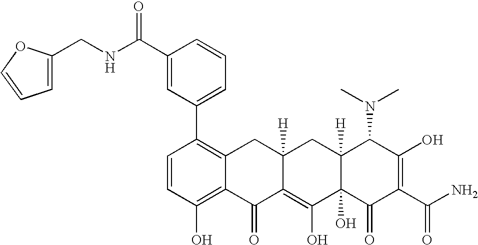 |
| Z | 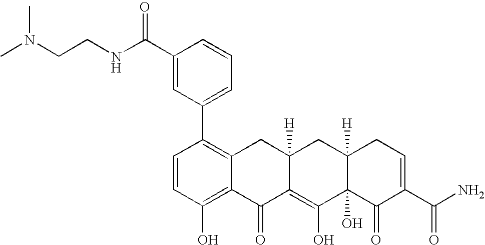 |
| AA | 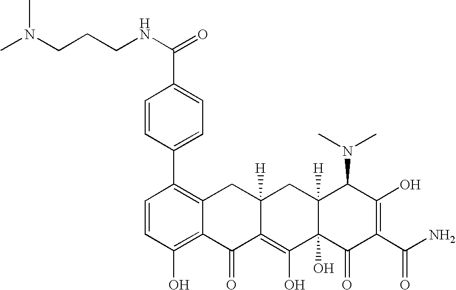 |
| AB | 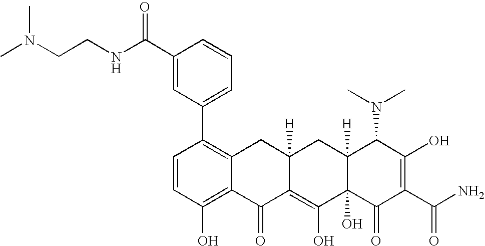 |
| AC | 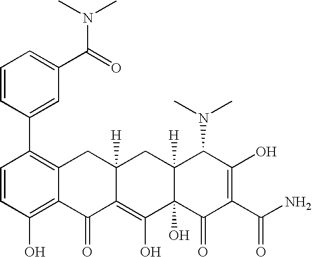 |
| AD | 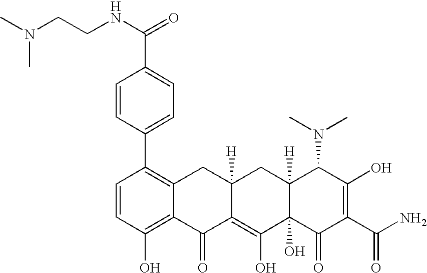 |
| AE | 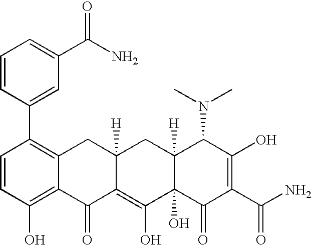 |
| AF | 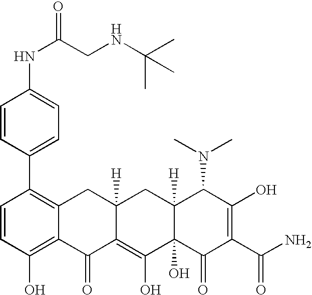 |
| AG | 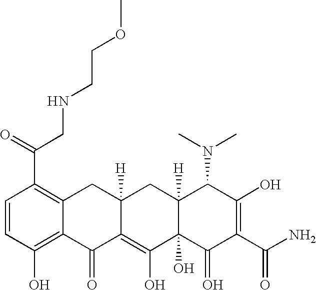 |
| AH | 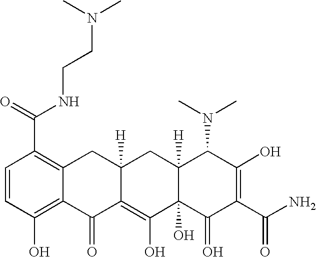 |
| AI | 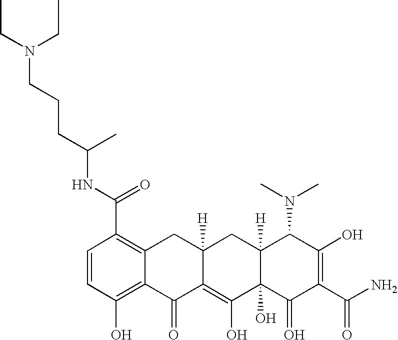 |
| AJ | 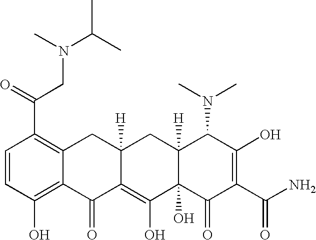 |
| AK | 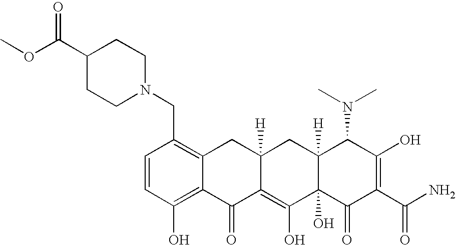 |
| AL | 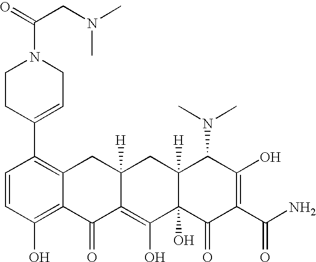 |
| AM | 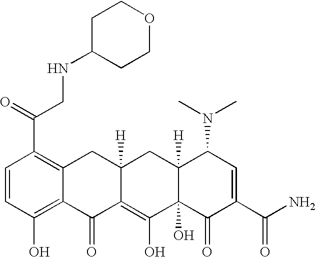 |
| AO | 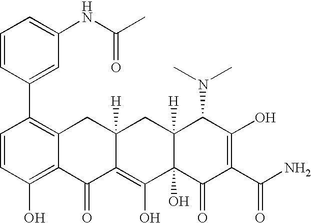 |
| AP | 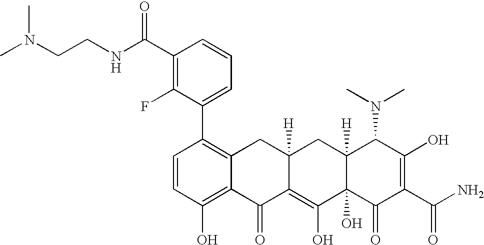 |
| AQ | 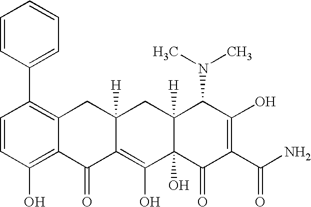 |
| AR | 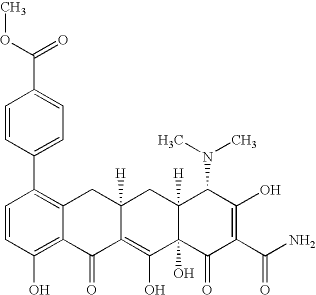 |
| AS | 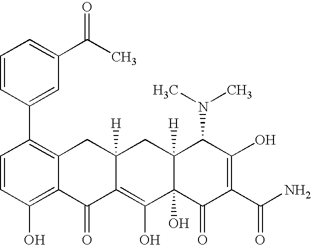 |
| AT | 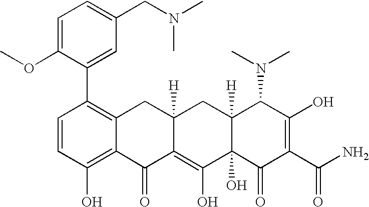 |
| AU | 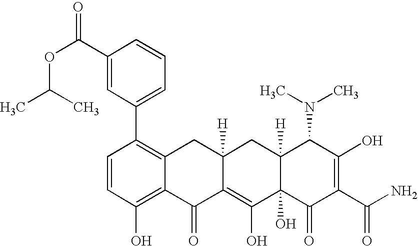 |
| AV | 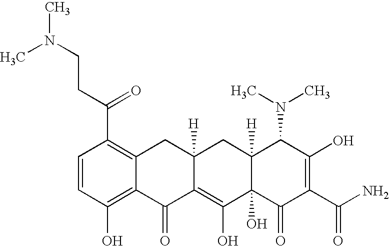 |
| AW | 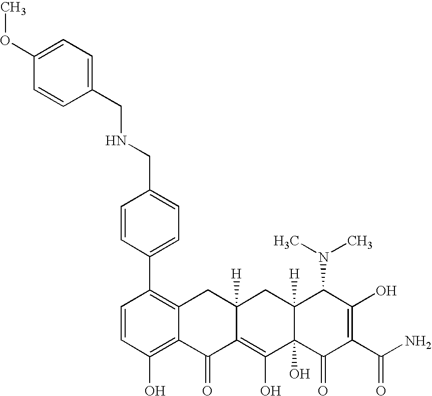 |
| AX | 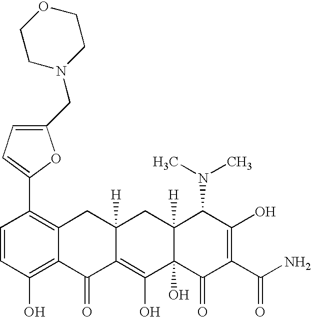 |
| AY | 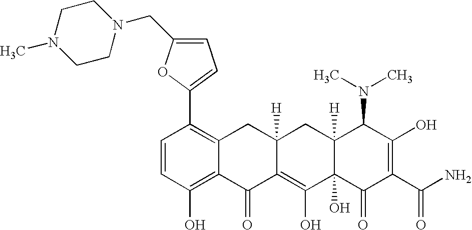 |
| AZ | 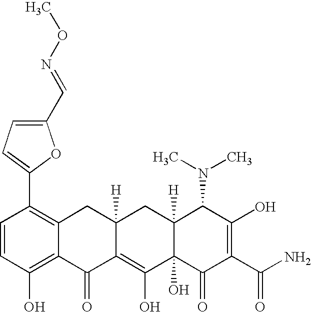 |
| BA | 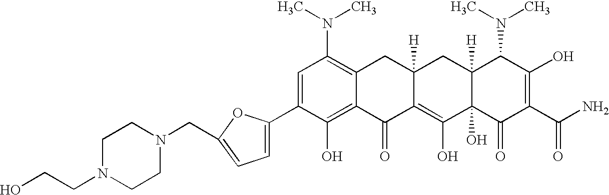 |
| BB | 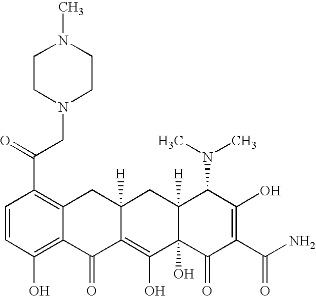 |
| BC | 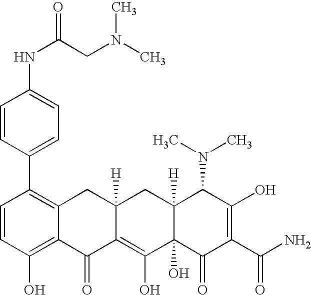 |
| BD | 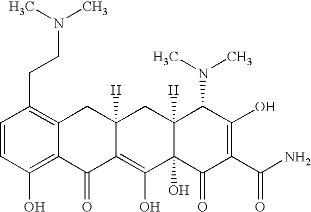 |
| BE | 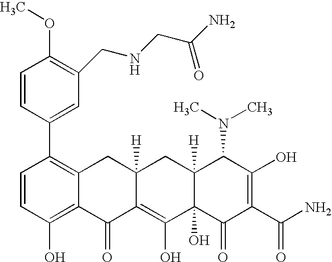 |
| BF | 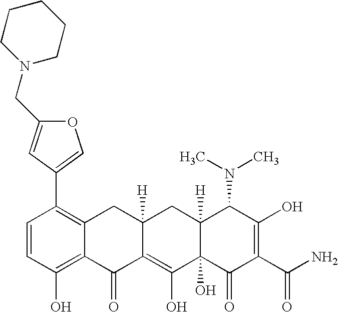 |
| BG | 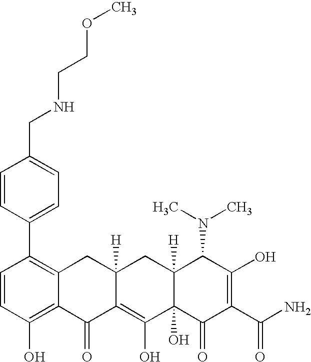 |
| BH | 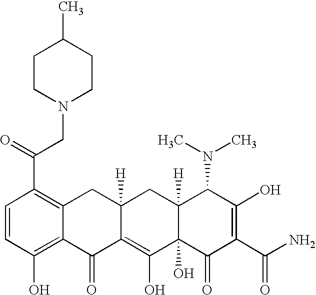 |
| BI | 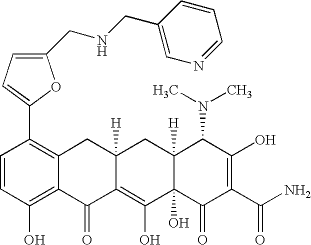 |
| BJ | 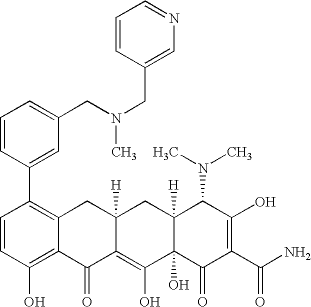 |
| BK | 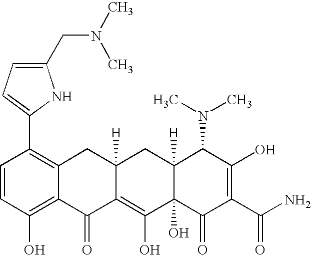 |
| BL | 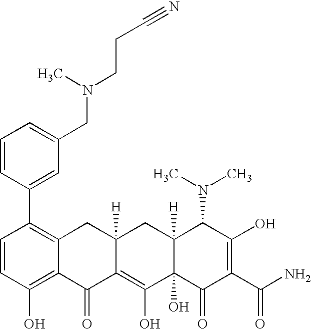 |
| BM | 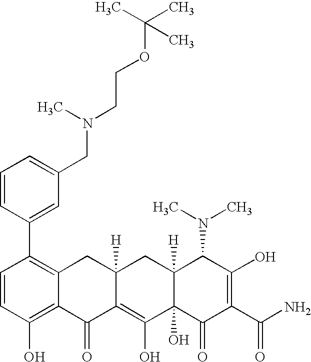 |
| BN | 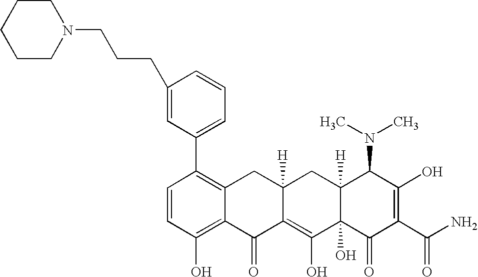 |
| BO | 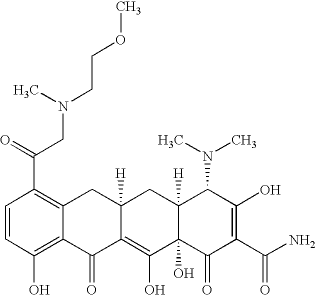 |
| BP | 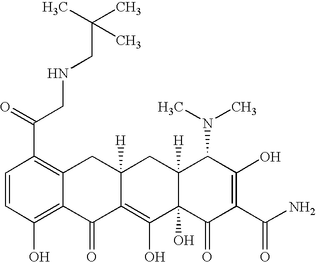 |
| BQ | 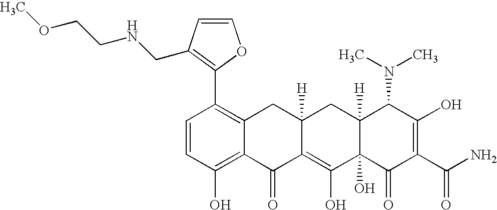 |
| BR | 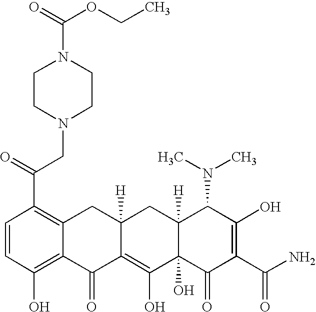 |
| BS | 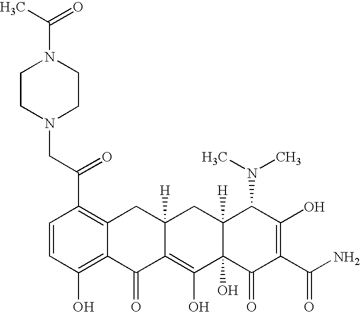 |
| BT | 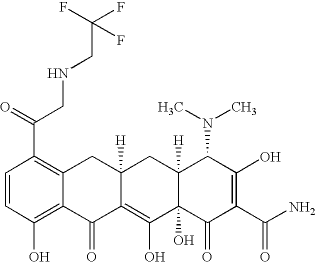 |
| BU | 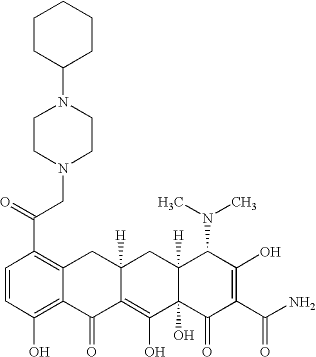 |
| BV | 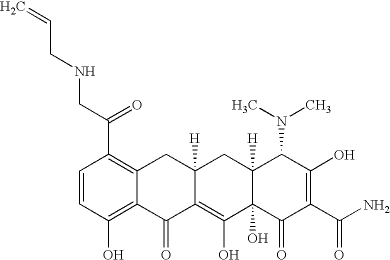 |
| BW | 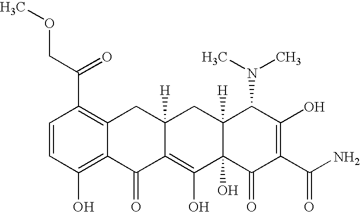 |
| BX | 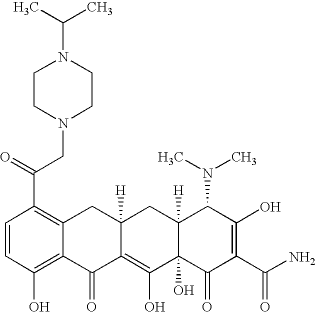 |
| BY | 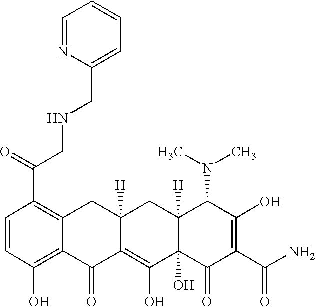 |
| BZ | 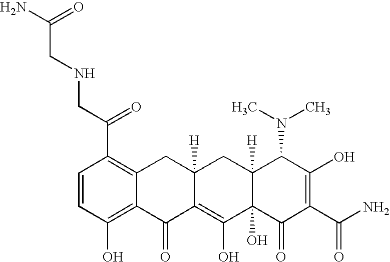 |
| CA | 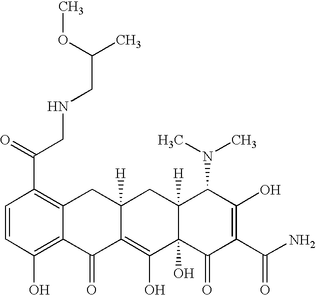 |
| CB | 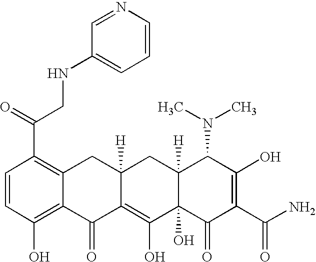 |
| CC | 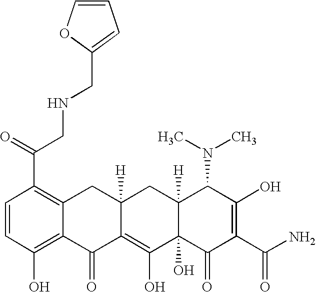 |
| CD | 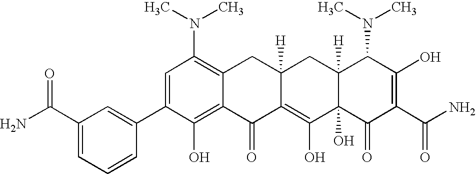 |
| CE | 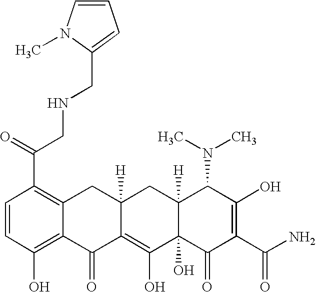 |
| CF | 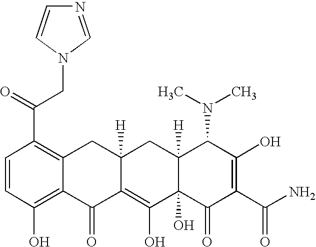 |
| CG | 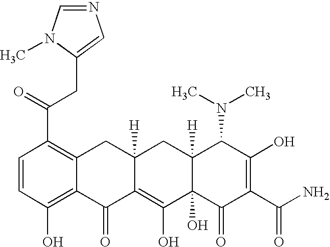 |
| CH | 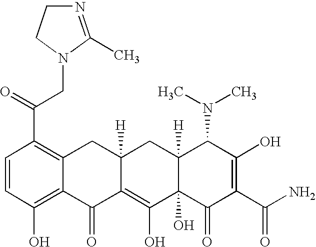 |
| CI | 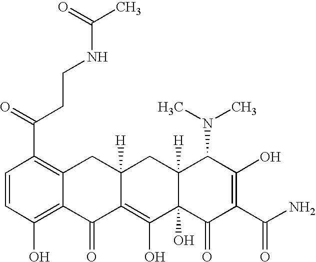 |
| CJ | 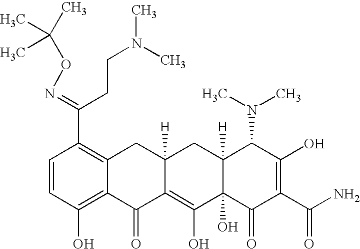 |
| CK | 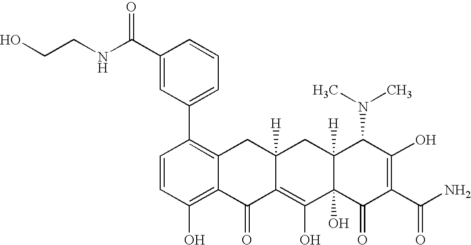 |
| CL | 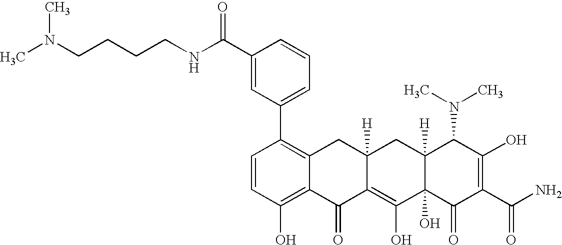 |
| CM | 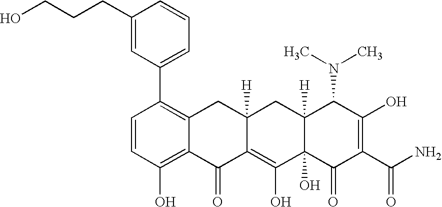 |
| CN | 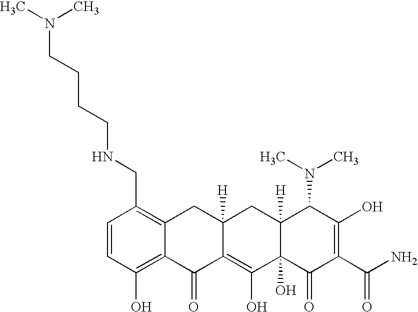 |
| CO | 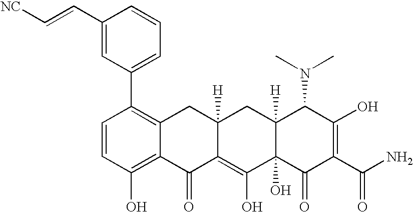 |
| CP | 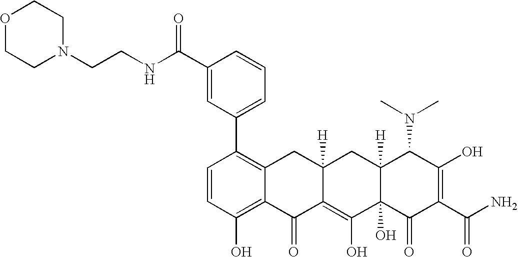 |
| CQ | 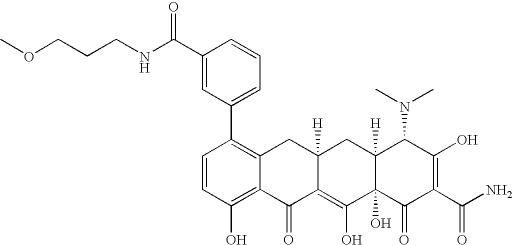 |
| CR | 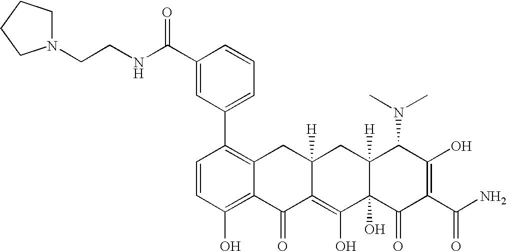 |
| CS | 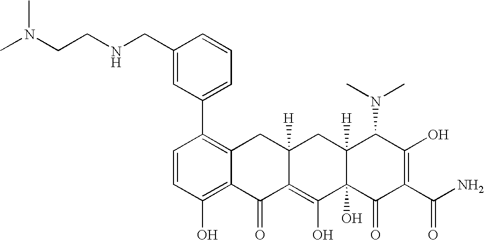 |
| CT | 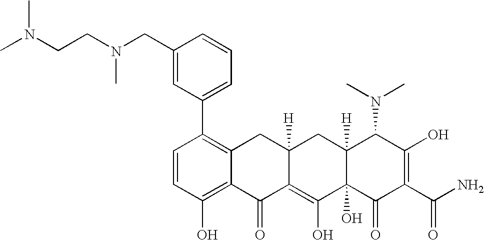 |
| CU | 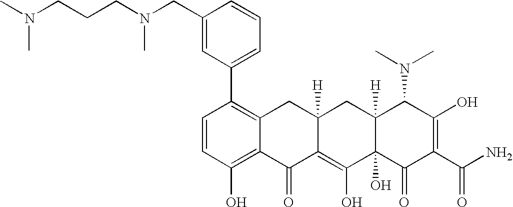 |
| CV | 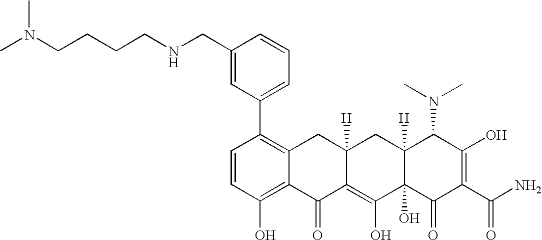 |
| CW | 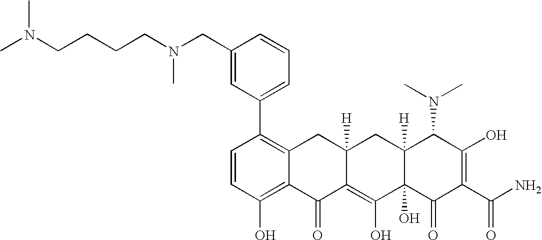 |
| CX | 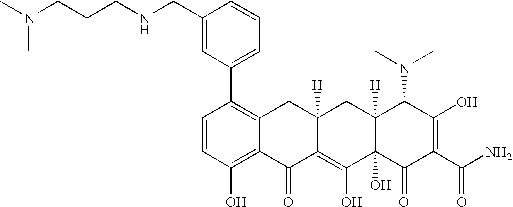 |
| CY | 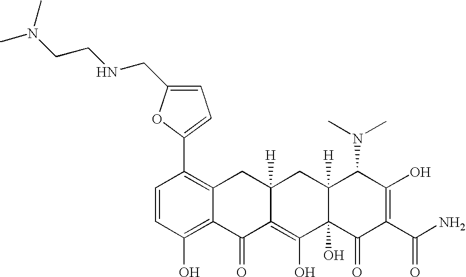 |
| CZ | 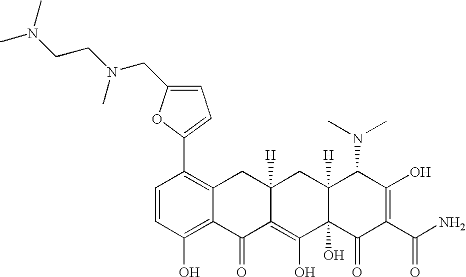 |
| DA | 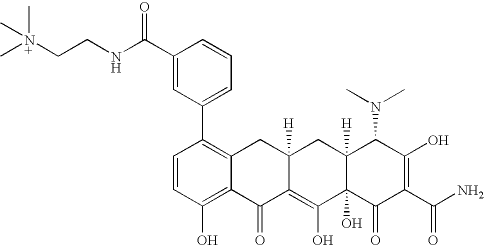 |
| DB | 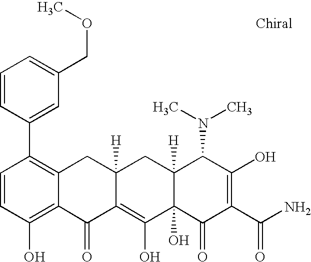 |
| DC | 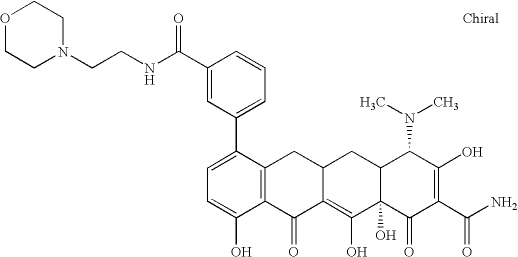 |
| DD | 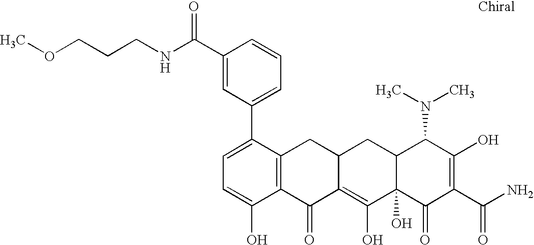 |
| DE | 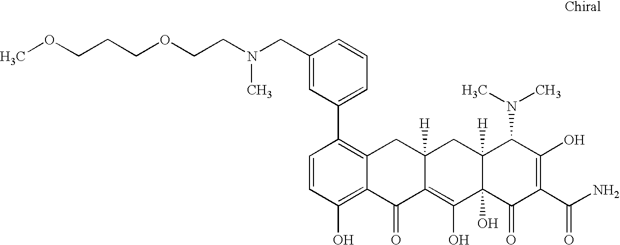 |
| DF | 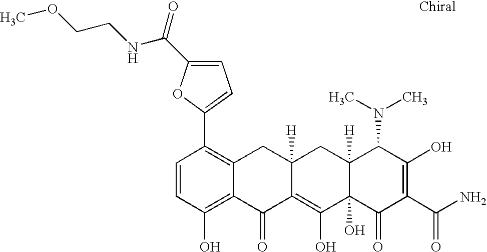 |
| DG | 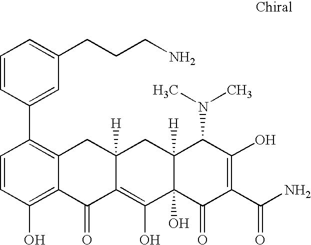 |
| DH | 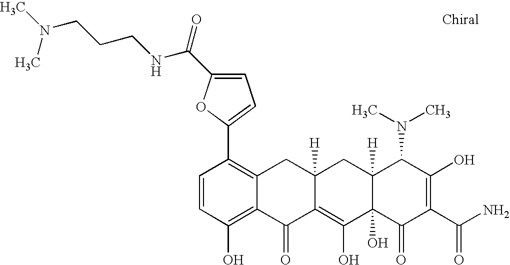 |
| DI | 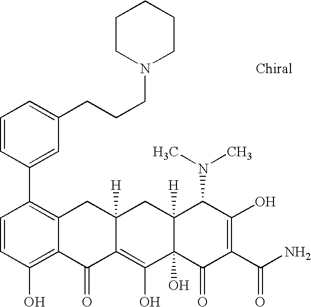 |
| DJ | 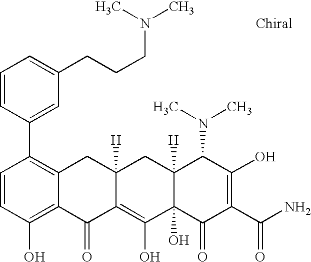 |
| DK | 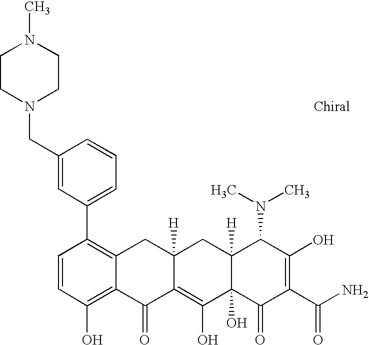 |

Each of the tetracycline compounds described herein may be used in the methods and pharmaceutical compositions of the invention.

### Methods for Synthesizing Tetracycline Compounds of the Invention.

The tetracycline compounds of the invention can be synthesized using the methods described in the following schemes and by using art recognized techniques.

Scheme 1 outlines the general synthesis of 7-substituted tetracyclines. A 7-iodo sancycline derivative (1) may be reacted in a Stille coupling or a Suzuki coupling by reacting with an organotin derivative or a boronic acid derivative in the presence of a palladium catalyst to form the desired product (2).

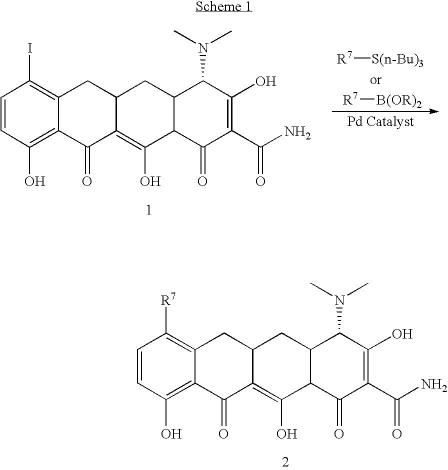

Scheme 2 depicts a method for synthesizing aromatic substituted 9-substituted tetracycline compounds. A 9-iodo tetracycline derivative (3) is reacted under Suzuki conditions by mixing with a boronic acid in the presence of the appropriate palladium catalyst to give compounds similar to compound 4. For example, compounds V, X, BA and CD may be synthesized as illustrated as in Scheme 2.

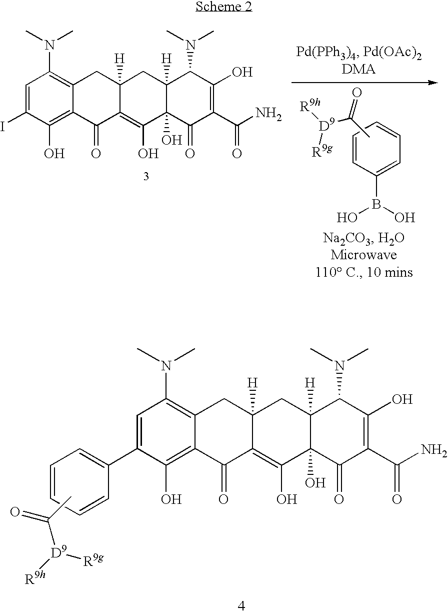

Scheme 3 depicts the synthesis of aminocarbonyl substituted aromatic 7-substituted-4-dedimethylamino tetracycline compounds. Starting from 7-iodo substituted-4-dedimethylamino sancycline (5), a Suzuki coupling reaction is performed with a boronic acid in the presence of a palladium catalyst to provide compound 6. For example, compounds B, Z and AE may be synthesized in this manner.

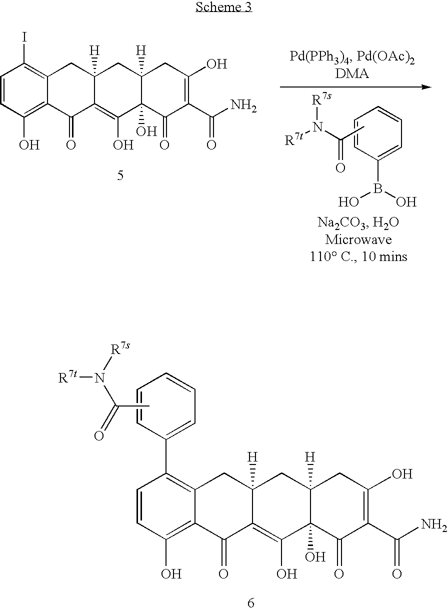

The 7-substituted acyl and oxime derivatives may also be prepared as shown in Scheme 4. An 7-iodo sancycline derivative (1) can be reacted with a substituted alkyne in the presence of palladium to synthesize the alkynyl derivative 7. Compound 7 may be converted to the acyl substituted compound 8 by any technique known in the art (e.g., by acid catalyzed hydrolysis). For example, compounds AV and CI may be prepared in this manner. The desired oxime product 9 can be obtained by reacting the acyl moiety with a primary hydroxylamine. For example, compound CJ may be synthesized as shown in Scheme 4.

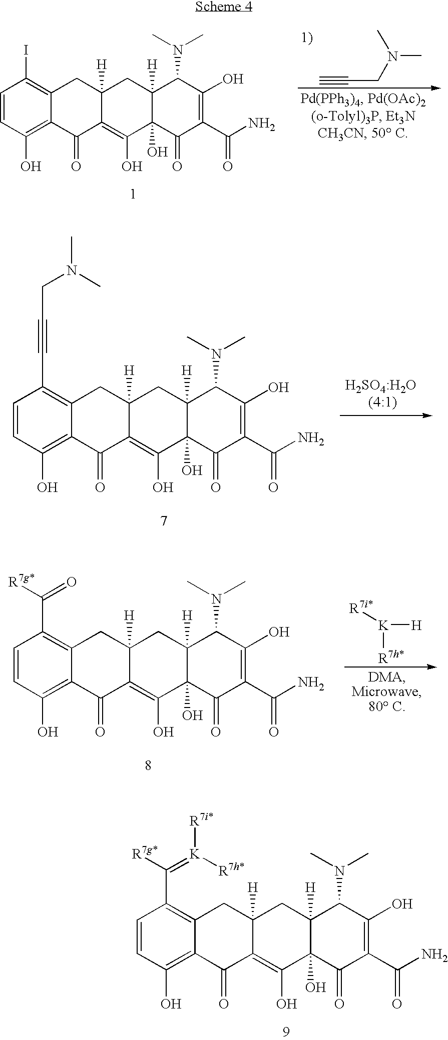

Scheme 5 depicts generally the synthesis of substituted aromatic 7-substituted tetracycline compounds. Beginning with 1 and performing a Suzuki coupling reaction in the presence of a boronic acid and a palladium catalyst, compounds of general formula 10 are formed. For example, compounds G, H, W, AQ, AR, AS, AT, AU, AW, BE, BG, BJ, BL, BM, BN, CM, BG and CO may be synthesized as shown in Scheme 5.

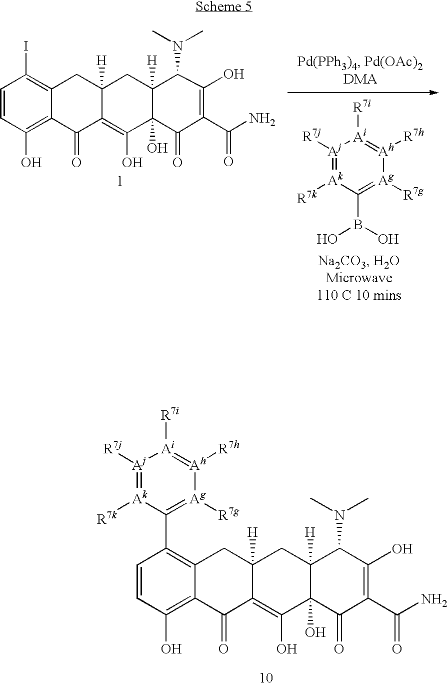

Scheme 6 also depicts the synthesis of substituted aromatic 7-substituted tetracycline compounds. Again, starting from 7-iodo substituted sancycline (1), a Suzuki coupling reaction is performed with a boronic acid in the presence of a palladium catalyst to provide intermediate 11 in which R⁷ⁱ or R^{7j} are either an amine or a carboxylic acid. If the substituent is a carboxylic acidic moiety, a coupling to a secondary amine in the presence of base and a typical coupling reagent to form 7-substituted tetracyclines similar to 12a. For example, compounds A, C, D, E, F, I, J, L, M, N, O, P, R, S, T, U, Y, Z, AB, AC, AD, AE, CK, CL and DA may be synthesized as illustrated in this manner. Alternatively, if the substituent is an amino moiety, coupling of the amino moiety to an acid chloride or carboxylic acid in the presence of a base and a typical coupling reagent may be used to form 7-substituted tetracyclines similar to 12b. For example, compounds K, Q, AO, AF and BC may be synthesized in this manner.

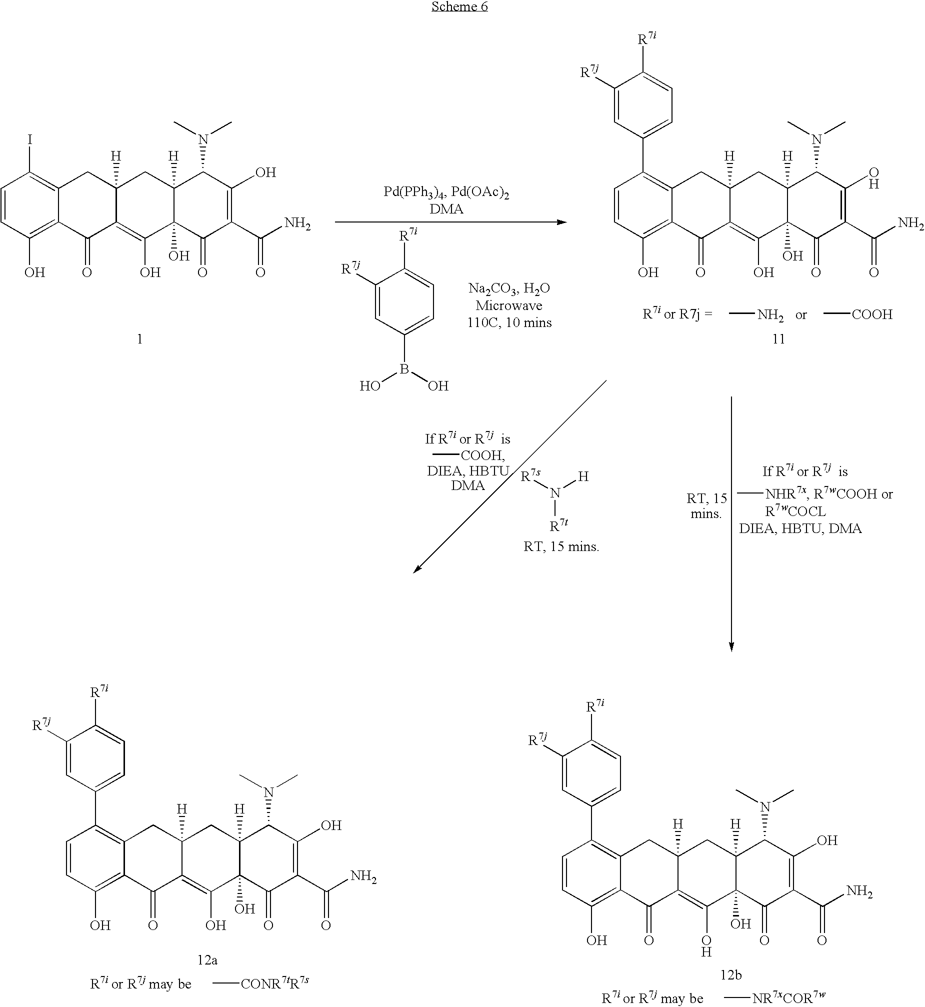

Synthesis of substituted 7-acyl tetracycline compounds may be accomplished by the general procedure outlined in Scheme 7. Alpha bromination of compound 13 yields the intermediate 14 which can be reacted with an appropriate nucleophile to yield compounds of the formula 15. For example, compounds AG, AJ, AM, BB, BH, BO, BP, BR, BS, BT, BU, BV, BW, BX, BY, BZ, CA, CB, CC, CE, CF and CH may be synthesized in this manner.

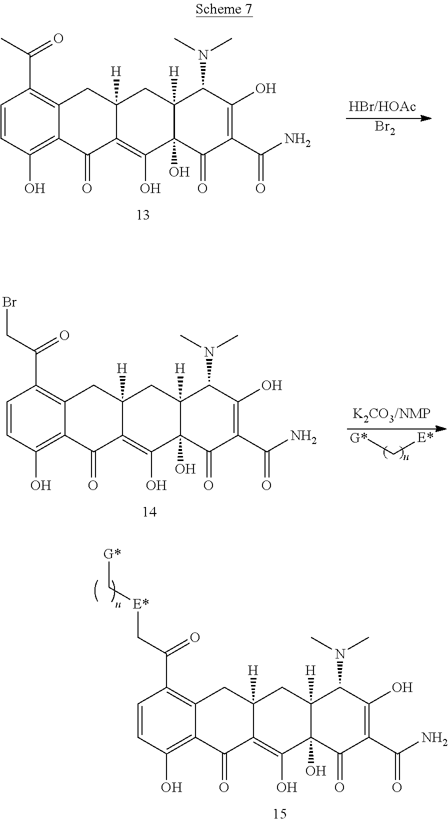

Substituted 7-carboxamide derivatives of tetracyclines may be prepared using the general synthesis outlined in Scheme 8. Carbonylation of the 7-iodotetracycline compound I yields the 7-carboxy tetracycline intermediate 16. Standard coupling reactions with the desired amine yields compounds of the formula 17. For example, compounds AH and AI may be synthesized in this manner.

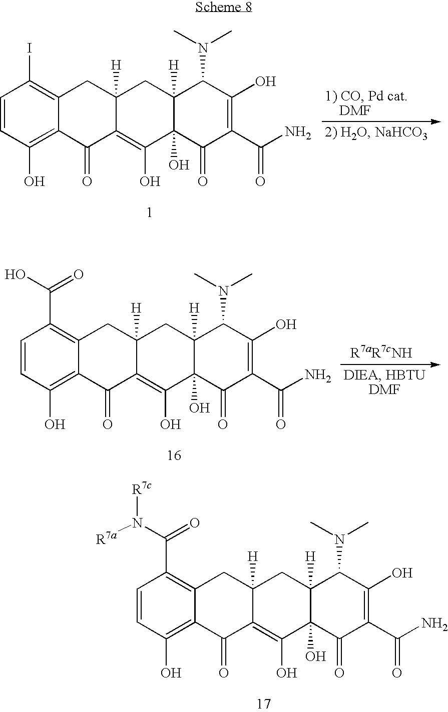

Scheme 9 illustrates the synthesis of 7-heteroaryl-substituted tetracycline derivatives. Using the general procedure outlined in Scheme 1, compounds of formula 18 may be prepared by performing a Suzuki coupling with a 2-formyl-heteroaryl boronic acid. Subsequent reaction of compounds of formula 18 with an amine or alkoxyamine yields the imine or oxime 19. This is the procedure used to synthesis AZ. Compound 19 may then be reduced to produce compounds of formula 20. For example, compounds AX, AY, BF, BI, BK, BQ, CY and CZ may be synthesized in this manner.

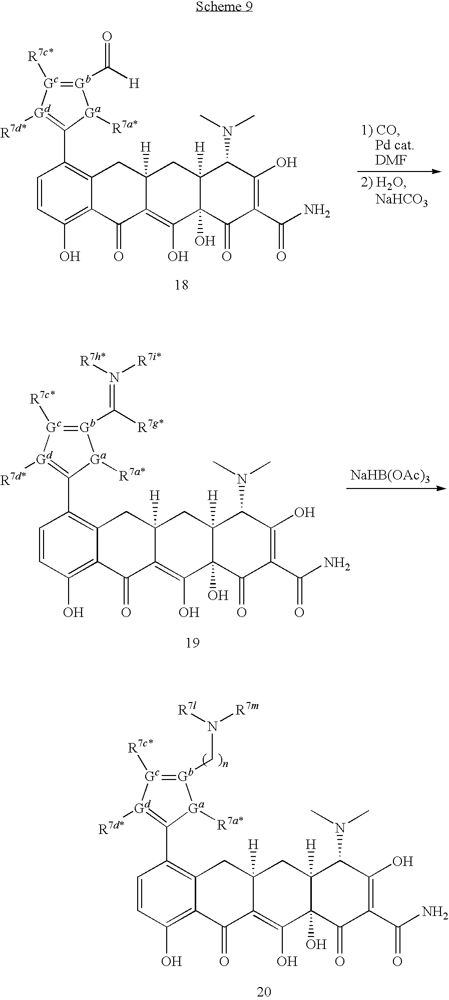

Scheme 10 describes the synthesis of 7-aminomethyl-substituted tetracyclines. Starting from compound 1, a carbonyl insertion reaction may be performed to yield the 7-formyl tetracycline 21. A reductive alkylation of compound 21 with an appropriate amine yields compounds of formula 22. For example, compounds AK and CN may be synthesized in this manner.

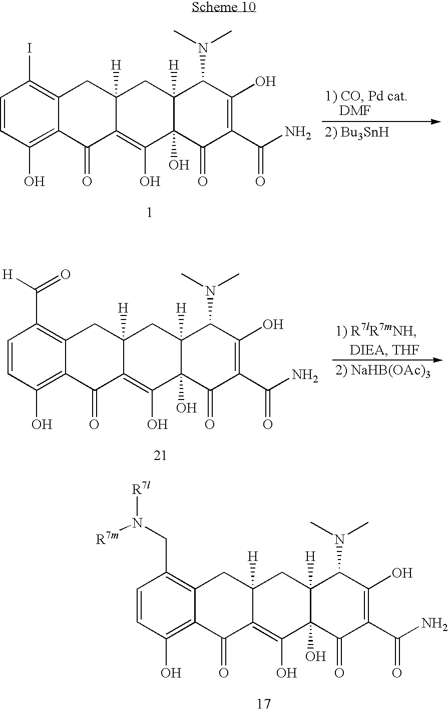

Scheme 11 describes the synthesis of 7-alkenyl-substituted tetracyclines via a Heck-type coupling. In this reaction, 7-iodotetracycline (1) is reacted with an appropriate alkene and appropriate palladium catalyst to yield the alkenyl-substituted compounds of formula 23. For example, compound AL may be synthesized in this manner.

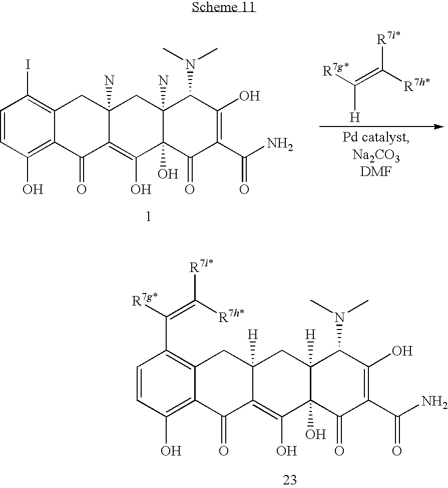

Scheme 12 depicts the synthesis of 7-(3-aminomethylphenyl)-tetracycline derivatives of formula 25. In this reaction, compound 24 (synthesized as described in Scheme 1), undergoes a reductive alkylation with an appropriate amine to yield compound 25. For example, compounds BJ, BL, BM, CS, CT, CU, CV, CW and CX may be synthesized in this manner.

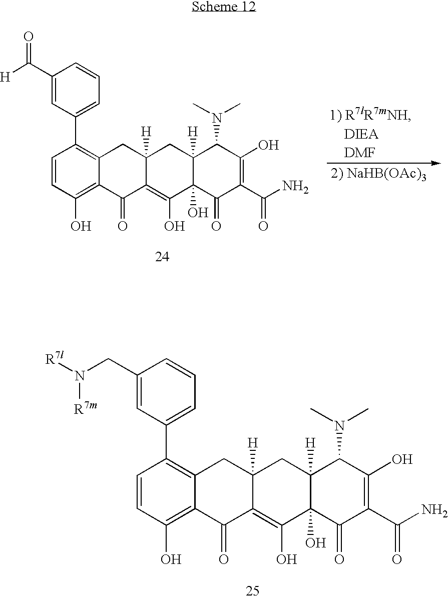

Scheme 13 describes the synthesis of 7-aminoethyl tetracycline derivatives similar to compound 28. 7-Iodotetracycline undergoes a Suzuki-type coupling with the appropriate boronic acid to yield compound 26, which is followed by an acid hydrolysis to yield aldehyde 27, which may further be modified by reductive alkylation to yield aminoethyl tetracyclines of formula 28. For example, compound BD may be synthesized in this manner.

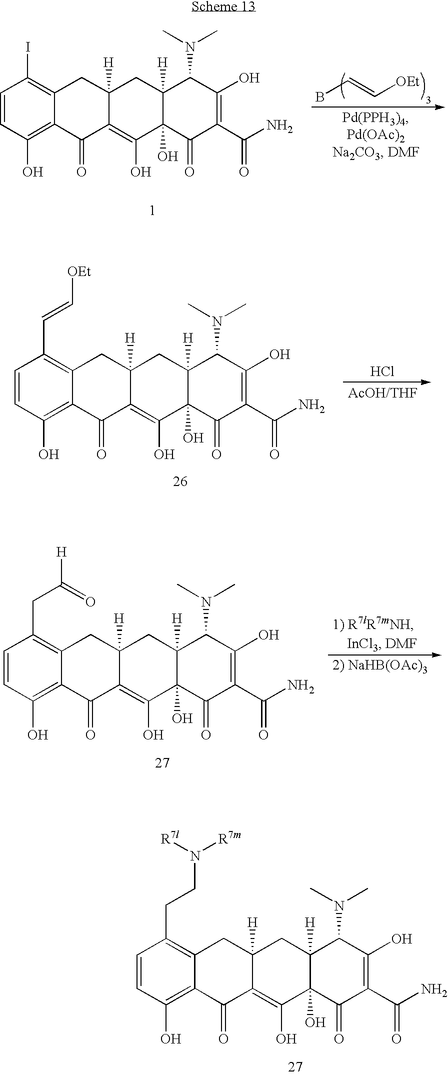

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (e.g., isopropyl, tert-butyl, isobutyl, etc.), cycloalkyl (alicyclic) groups (e.g., cyclopropyl, cyclobutyl, cyclopentyl, cyclohexyl, cycloheptyl, cyclooctyl), alkyl substituted cycloalkyl groups, and cycloalkyl substituted alkyl groups. The term alkyl can include heteroalkyl groups that include oxygen, nitrogen, sulfur or phosphorous atoms replacing one or more carbons of the hydrocarbon backbone. In certain embodiments, a straight chain or branched chain alkyl has 20 or fewer carbon atoms in its backbone (e.g., C₁-C₂₀ for straight chain, C₃-C₂₀ for branched chain), and more preferably 4 or fewer. Cycloalkyls may have from 3-8 carbon atoms in their ring structure, and more preferably have 5 or 6 carbons in the ring structure. The term C₁-C₆ includes alkyl groups containing 1 to 6 carbon atoms.

The term “heterocyclic” includes cycloalkyl moieties in which one or more carbons of the cycloalkyl scaffold is replace with a heteroatom, for example, oxygen, nitrogen, sulfur or phosphorous. Examples of heterocyclic moieties include piperidine, morpholine, pyrrolidine, piperazine and tetrahydrofuran.

“Unsubstituted alkyls” refers to alkyl moieties having no substituents replacing a hydrogen on one or more carbons of the hydrocarbon backbone.

“Substituted alkyls” refers to alkyl moieties having one or more substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be substituted, e.g., with the substituents described above. An “alkylaryl” or an “arylalkyl” moiety is an alkyl substituted with an aryl (e.g., phenylmethyl (benzyl)).

The term “aryl” includes groups, including 5- and 6-membered single-ring aromatic groups that may include from zero to four heteroatoms, for example, benzene, phenyl, pyrrole, furan, thiophene, thiazole, isothiazole, imidazole, triazole, tetrazole, pyrazole, oxazole, isooxazole, pyridine, tetrahydropyridine, quinoline, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxophenyl, quinoline, isoquinoline, naphthridine, indole, benzofuran, purine, benzofuran, deazapurine, or indolizine. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulflhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond.

For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl can include alkenyl groups which include oxygen, nitrogen, sulfur or phosphorous atoms replacing one or more carbons of the hydrocarbon backbone. In certain embodiments, a straight chain or branched chain alkenyl group has 20 or fewer carbon atoms in its backbone (e.g., C₂-C₂₀ for straight chain, C₃-C₂₀ for branched chain). Likewise, cycloalkenyl groups may have from 3-8 carbon atoms in their ring structure, and more preferably have 5 or 6 carbons in the ring structure. The term C₂-C₂₀ includes alkenyl groups containing 2 to 20 carbon atoms.

“Unsubstituted alkenyls” refers to alkenyl moieties having no substituents replacing a hydrogen on one or more carbons of the hydrocarbon backbone.

“Substituted alkenyls” refers to alkenyl moieties having one or more substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl can include alkynyl groups which include oxygen, nitrogen, sulfur or phosphorous atoms replacing one or more carbons of the hydrocarbon backbone. In certain embodiments, a straight chain or branched chain alkynyl group has 20 or fewer carbon atoms in its backbone (e.g., C₂-C₂₀ for straight chain, C₃-C₂₀ for branched chain). The term C₂-C₆ includes alkynyl groups containing 2 to 6 carbon atoms.

“Unsubstituted alkynyls” refers to alkynyl moieties having no substituents replacing a hydrogen on one or more carbons of the hydrocarbon backbone.

“Substituted alkynyls” refers to alkynyl moieties having one or more substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including, e.g., alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, two to five carbon atoms.

The term “acyl” includes compounds and moieties which contain the acyl radical (CH₃CO—). The term “substituted acyl” includes acyl groups where one or more of the hydrogen atoms are replaced by for example, alkyl groups, alkenyl, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “carbonylamino” includes moieties wherein a carbonyl moiety (e.g., —C(═O)) is bonded to an amino group. For example, the term includes alkylcarbonylamino, arylcarbonylamino, carbamoyl and ureido groups.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The terms “alkoxyalkyl,” “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “amide” or “aminocarbonyl” includes compounds or moieties which contain a nitrogen atom which is bound to the carbon of a carbonyl or a thiocarbonyl group. The term includes “arylaminocarbonyl” groups which include aryl or heteroaryl moieties bound to an amino group which is bound to the carbon of a carbonyl or thiocarbonyl group. The term also includes “alkylaminocarboxy,” “alkenylaminocarboxy,” “alkynylaminocarboxy,” and in which alkyl, alkenyl and alkynyl moieties, respectively, are bound to a nitrogen atom which is in turn bound to the carbon of a carbonyl group.

The term “amine” or “amino” includes compounds where a nitrogen atom is covalently bonded to at least one carbon or heteroatom. The term includes “alkylamino” moieties, wherein the nitrogen is bound to at least one additional alkyl group. The term also includes “dialkylamino” groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “aroyl” includes compounds and moieties with an aryl or heteroaromatic moiety bound to a carbonyl group. Examples of aroyl groups include phenylcarboxy, naphthyl carboxy, etc.

The term “carbonyl” or “carboxy” includes compounds and moieties which contain a carbon connected with a double bond to an oxygen atom and the carbon atom is bonded to two additional moieties. Examples of moieties which contain a carbonyl include aldehydes, ketones, carboxylic acids, amides, esters, anhydrides, etc. Suitable moieties bonded to the carbon of a carbonyl group include, for example, hydrogen, alkyl groups, alkenyl, alkynyl groups, halogens, hydroxyl, alkylcarb onyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “carbonyloxy” includes moieties in which the carbon of a carbonyl group is covalently bound to an oxygen.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “halogen” includes fluorine, bromine, chlorine, iodine, etc. The term “perhalogenated” generally refers to a moiety wherein all hydrogens are replaced by halogen atoms.

The term “heteroatom” includes atoms of any element other than carbon or hydrogen. Preferred heteroatoms are nitrogen, oxygen, sulfur and phosphorus.

The term “hydroxy” or “hydroxyl” includes groups with an —OH or —O⁻X⁺, where X⁺ is a counterion.

The terms “polycyclyl” or “polycyclic radical” refer to two or more cyclic rings (e.g., cycloalkyls, cycloalkenyls, cycloalkynyls, aryls and/or heterocyclyls) in which two or more carbons are common to two adjoining rings, e.g., the rings are “fused rings.” Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, arylalkylaminocarbonyl, alkenylaminocarbonyl, alkylcarbonyl, arylcarbonyl, arylalkyl carbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “thiocarbonyl” or “thiocarboxy” includes compounds and moieties which contain a carbon connected with a double bond to a sulfur atom.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “sulfonyl” includes moieties which comprise a sulfonyl group. Similarly, the term “sulfinyl” includes moieties which comprise a sulfinyl group.

The term “oximyl” includes moieties which comprise an oxime group.

The term “dimeric moiety” includes moieties which comprise a second tetracycline four ring structure. The dimeric moiety may be attached to the substituted tetracycline through a chain of from 1-30 atoms. The chain may be comprised of atoms covalently linked together through single, double and triple bonds. The tetracycline ring structure of the dimeric moiety may further be substituted or unsubstituted. It may be attached at the 1, 2, 3, 4, 5, 6, 7, 8, 9, 10, 11, 11a, 12, 12a, and/or 13 position.

The term “prodrug moiety” includes moieties which can be metabolized in vivo. Generally, the prodrugs moieties are metabolized in vivo by esterases or by other mechanisms to hydroxyl groups or other advantageous groups. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The structures of some of the substituted tetracycline compounds used in the methods and compositions of the invention include asymmetric carbon atoms. The isomers arising from the chiral atoms (e.g., all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof.

### Methods for Treating Bacterial Infections

The invention pertains to methods for treating a microorganism-associated infection in a subject, by administering to a subject an effective amount of a tetracycline compound of the invention (e.g., a compound of Formula I, II, III, IV, V, VI, VII, VIII, IX or X or a compound listed in Table 2), such that the microorganism-associated infection is treated.

The term “treating” or “treat” includes ameliorating at least one symptom of the state, disease or disorder, e.g., the microorganism-associated infection. In one embodiment, the term “treating” includes curing at least one symptom of the state, disease or disorder, e.g., the microorganism-associated infection.

The term “preventing” or “prevent” describes reducing or eliminating the onset of the symptoms or complications of the microorganism-associated infection.

The tetracycline compounds of the present invention can be used to treat a microorganism-associated infection, including bacterial, viral, parasitic, or a fungal infection (including those which are resistant to other tetracycline compounds). Compounds of the invention can be used to prevent or treat important mammalian and veterinary diseases such as diarrhea caused by a microorganism-associated infection, urinary tract infections, infections of skin and skin structure, ear, nose and throat infections, wound infection, mastitis and the like.

The compounds described herein may be used in combination with another therapeutic agent or treatment to treat or prevent a microorganism-associated infection.

The language “in combination with” another therapeutic agent or treatment includes co-administration of the tetracycline compound, (e.g., inhibitor) and with the other therapeutic agent or treatment, administration of the tetracycline compound first, followed by the other therapeutic agent or treatment and administration of the other therapeutic agent or treatment first, followed by the tetracycline compound. The other therapeutic agent may be any agent that is known in the art to treat, prevent, or reduce the symptoms of a particular infection. Furthermore, the other therapeutic agent may be any agent of benefit to the patient when administered in combination with the administration of a tetracycline compound.

Bacterial infections may be caused by a wide variety of gram positive and gram negative bacteria. Some of the compounds of the invention are useful as antibiotics against organisms which are resistant and/or sensitive to other tetracycline compounds. The antibiotic activity of the tetracycline compounds of the invention may by using the in vitro standard broth dilution method described in Waitz, J. A., CLSI, Document M7-A2, vol. 10, no. 8, pp. 13-20, 2^{nd} edition, Villanova, Pa. (1990).

The tetracycline compounds may also be used to treat infections traditionally treated with tetracycline compounds such as, for example, a microorganism-associated infection, caused by, e.g., rickettsiae; a number of gram-positive and gram-negative bacteria; or the agents responsible for lymphogranuloma venereum, inclusion conjunctivitis, or psittacosis. The tetracycline compounds may be used to treat infections of, e.g., K. pneumoniae, Salmonella, E. hirae, A. baumanii, B. catarrhalis, H. influenzae, P. aeruginosa, E. faecium, E. coli, S. aureus or E. faecalis. In one embodiment, the tetracycline compound is used to treat a microorganism-associated infection that is resistant to other tetracycline antibiotic compounds. The tetracycline compound of the invention may be administered with a pharmaceutically acceptable carrier.

The language “effective amount” of the compound is that amount necessary or sufficient to treat a microorganism-associated infection (e.g., bacterial infection, viral infection, parasitic infection or fungal infection).

Alternatively, an “effective amount” of the compound is that amount necessary or sufficient to prevent onset of a microorganism-associated infection (e.g., bacterial infection, viral infection, parasitic infection or fungal infection).

The effective amount can vary depending on such factors as the size and weight of the subject, the type of illness, or the particular tetracycline compound. For example, the choice of the tetracycline compound can affect what constitutes an “effective amount.” One of ordinary skill in the art would be able to study the aforementioned factors and make the determination regarding the effective amount of the tetracycline compound without undue experimentation.

The invention pertains to methods of treatment against microorganism infections and associated diseases. The methods include administration of an effective amount of one or more tetracycline compounds to a subject. The subject can be either a plant or, advantageously, an animal, e.g., a mammal, e.g., a human.

In the therapeutic methods of the invention, one or more tetracycline compounds of the invention may be administered alone to a subject, or more typically a compound of the invention will be administered as part of a pharmaceutical composition in mixture with conventional excipient, i.e., pharmaceutically acceptable organic or inorganic carrier substances suitable for parenteral, oral or other desired administration and which do not deleteriously react with the active compounds and are not deleterious to the recipient thereof.

### Pharmaceutical Compositions of the Invention

The invention also pertains to pharmaceutical compositions comprising a therapeutically effective amount of a tetracycline compound (e.g., a compound of Formula I, II, III, IV, V, VI, VII, VIII, IX or X or a compound listed in Table 2) and, optionally, a pharmaceutically acceptable carrier.

The language “pharmaceutically acceptable carrier” includes substances capable of being coadministered with the tetracycline compound(s), and which allow both to perform their intended function, e.g., treat a microorganism-associated infection (e.g., bacterial infection, viral infection, parasitic infection or fungal infection).

Alternatively, a “pharmaceutically acceptable carrier” includes substances capable of being coadministered with the tetracycline compound(s), and which allow both to perform their intended function, e.g., prevent a microorganism-associated infection (e.g., bacterial infection, viral infection, parasitic infection or fungal infection).

Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds of the invention.

The tetracycline compounds of the invention that are basic in nature are capable of forming a wide variety of salts with various inorganic and organic acids. The acids that may be used to prepare pharmaceutically acceptable acid addition salts of the tetracycline compounds of the invention that are basic in nature are those that form non-toxic acid addition salts, i.e., salts containing pharmaceutically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and palmoate [i.e., 1,1′-methylene-bis-(2-hydroxy-3-naphthoate)] salts. Although such salts must be pharmaceutically acceptable for administration to a subject, e.g., a mammal, it is often desirable in practice to initially isolate a tetracycline compound of the invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The preparation of other tetracycline compounds of the invention not specifically described in the foregoing experimental section can be accomplished using combinations of the reactions described above that will be apparent to those skilled in the art.

The preparation of other tetracycline compounds of the invention not specifically described in the foregoing experimental section can be accomplished using combinations of the reactions described above that will be apparent to those skilled in the art.

The tetracycline compounds of the invention that are acidic in nature are capable of forming a wide variety of base salts. The chemical bases that may be used as reagents to prepare pharmaceutically acceptable base salts of those tetracycline compounds of the invention that are acidic in nature are those that form non-toxic base salts with such compounds. Such non-toxic base salts include, but are not limited to those derived from such pharmaceutically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. The pharmaceutically acceptable base addition salts of tetracycline compounds of the invention that are acidic in nature may be formed with pharmaceutically acceptable cations by conventional methods. Thus, these salts may be readily prepared by treating the tetracycline compound of the invention with an aqueous solution of the desired pharmaceutically acceptable cation and evaporating the resulting solution to dryness, preferably under reduced pressure. Alternatively, a lower alkyl alcohol solution of the tetracycline compound of the invention may be mixed with an alkoxide of the desired metal and the solution subsequently evaporated to dryness.

The preparation of other tetracycline compounds of the invention not specifically described in the foregoing experimental section can be accomplished using combinations of the reactions described above that will be apparent to those skilled in the art.

The tetracycline compounds of the invention and pharmaceutically acceptable salts thereof can be administered via either the oral, parenteral or topical routes. In general, these compounds are most desirably administered in effective dosages, depending upon the weight and condition of the subject being treated and the particular route of administration chosen. Variations may occur depending upon the species of the subject being treated and its individual response to said medicament, as well as on the type of pharmaceutical formulation chosen and the time period and interval at which such administration is carried out.

The pharmaceutical compositions of the invention may be administered alone or in combination with other known compositions for treating microorganism-associated infections in a subject, e.g., a mammal. Preferred mammals include pets (e.g., cats, dogs, ferrets, etc.), farm animals (cows, sheep, pigs, horses, goats, etc.), lab animals (rats, mice, monkeys, etc.), and primates (chimpanzees, humans, gorillas). The language “in combination with” a known composition is intended to include simultaneous administration of the composition of the invention and the known composition, administration of the composition of the invention first, followed by the known composition and administration of the known composition first, followed by the composition of the invention.

The tetracycline compounds of the invention may be administered alone or in combination with pharmaceutically acceptable carriers or diluents by any of the routes previously mentioned, and the administration may be carried out in single or multiple doses. For example, the novel therapeutic agents of this invention can be administered advantageously in a wide variety of different dosage forms, i.e., they may be combined with various pharmaceutically acceptable inert carriers in the form of tablets, capsules, lozenges, troches, hard candies, powders, sprays (e.g., aerosols, etc.), creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the therapeutically-effective compounds of this invention are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof. The compositions of the invention may be formulated such that the tetracycline compositions are released over a period of time after administration.

For parenteral administration (including intraperitoneal, subcutaneous, intravenous, intradermal or intramuscular injection), solutions of a therapeutic compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art. For parenteral application, examples of suitable preparations include solutions, preferably oily or aqueous solutions as well as suspensions, emulsions, or implants, including suppositories. Therapeutic compounds may be formulated in sterile form in multiple or single dose formats such as being dispersed in a fluid carrier such as sterile physiological saline or 5% saline dextrose solutions commonly used with injectables.

Additionally, it is also possible to administer the compounds of the present invention topically when treating inflammatory conditions of the skin. Examples of methods of topical administration include transdermal, buccal or sublingual application. For topical applications, therapeutic compounds can be suitably admixed in a pharmacologically inert topical carrier such as a gel, an ointment, a lotion or a cream. Such topical carriers include water, glycerol, alcohol, propylene glycol, fatty alcohols, triglycerides, fatty acid esters, or mineral oils. Other possible topical carriers are liquid petrolatum, isopropylpalmitate, polyethylene glycol, ethanol 95%, polyoxyethylene monolauriate 5% in water, sodium lauryl sulfate 5% in water, and the like. In addition, materials such as anti-oxidants, humectants, viscosity stabilizers and the like also may be added if desired.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc.

In addition to treatment of human subjects, the therapeutic methods of the invention also will have significant veterinary applications, e.g., for treatment of livestock such as cattle, sheep, goats, cows, swine and the like; poultry such as chickens, ducks, geese, turkeys and the like; horses; and pets such as dogs and cats. Also, the compounds of the invention may be used to treat non-animal subjects, such as plants.

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests conducted with regard to the foregoing guidelines.

In general, compounds of the invention for treatment can be administered to a subject in dosages used in prior tetracycline therapies. See, for example, the Physicians' Desk Reference. For example, a suitable effective dose of one or more compounds of the invention will be in the range of from 0.01 to 100 milligrams per kilogram of body weight of recipient per day, preferably in the range of from 0.1 to 50 milligrams per kilogram body weight of recipient per day, more preferably in the range of 1 to 20 milligrams per kilogram body weight of recipient per day. The desired dose is suitably administered once daily, or several sub-doses, e.g., 2 to 5 sub-doses, are administered at appropriate intervals through the day, or other appropriate schedule.

It will also be understood that normal, conventionally known precautions will be taken regarding the administration of tetracyclines generally to ensure their efficacy under normal use circumstances. Especially when employed for therapeutic treatment of humans and animals in vivo, the practitioner should take all sensible precautions to avoid conventionally known contradictions and toxic effects. Thus, the conventionally recognized adverse reactions of gastrointestinal distress and inflammations, the renal toxicity, hypersensitivity reactions, changes in blood, and impairment of absorption through aluminum, calcium, and magnesium ions should be duly considered in the conventional manner.

Furthermore, the invention also pertains to the use of a tetracycline compound of Formula I, II, III, IV, V, VI, VII, VIII, IX or X or a compound listed in Table 2, or any other compound described herein, for the preparation of a medicament. The medicament may include a pharmaceutically acceptable carrier and the tetracycline compound is an effective amount, e.g., an effective amount to treat a microorganism-associated infection.

The invention is further illustrated by the following examples, which should not be construed as further limiting. The contents of all references, pending patent applications and published patents, cited throughout this application are hereby expressly incorporated by reference.

### EXEMPLIFICATION OF THE INVENTION

### Example 1

### In Vitro Anti-Bacterial Activity Assay

The following assay was used to determine the efficacy of the tetracycline compounds against gram positive and gram negative bacteria. 2 mg of each compound was dissolved in 100 μl of DMSO. The solution was then added to cation-adjusted Mueller Hinton broth (CAMHB), which resulted in a final compound concentration of 200 μg per ml. The tetracycline compound solutions were diluted to 50 μL volumes, with a test compound concentration of 0.098 μg/ml. Optical density (OD) determinations were made from fresh log-phase broth cultures of the test strains. Dilutions were made to achieve a final cell density of 1×10 CFU/ml. At OD=1, cell densities for different genera were approximately:

| | |
|---|---|
| E. coli | 1 × 10⁹ CFU/ml |
| S. aureus | 5 × 10⁸ CFU/ml |

50 μl of the cell suspensions were added to each well of microtiter plates. The final cell density was approximately 5×10⁵ CFU/ml. These plates were incubated at 35° C. in an ambient air incubator for approximately 18 hours. The plates were read with a microplate reader and were visually inspected when necessary. The MIC was defined as the lowest concentration of the tetracycline compound that inhibits growth. Table 3 includes MIC data for several substituted tetracycline compounds.

### Example 2

### Mammalian Cytotoxicity Assay

COS-1 and CHO-K1 cell suspensions were prepared, seeded into 96-well tissue culture treated black-walled microtiter plates (density determined by cell line), and incubated overnight at 37° C., in 5% CO₂ and approximately 95% humidity. The following day, serial dilutions of compound were prepared under sterile conditions and transferred to cell plates. Cell/Compound plates were incubated under the above conditions for 24 hours. Following the incubation period, media/compound was aspirated and 50 μl of resazurin (0.042 mg/ml in PBS w/Ca and Mg) is added. The plates were then incubated under the above conditions for 2 hours and then placed in the dark at room temperature for an additional 30 minutes. Fluorescence measurements were taken (excitation 535 nm, emission 590 nm). The IC₅₀ (concentration of compound causing 50% growth inhibition) was then calculated. Table 3 includes IC₅₀ data for several substituted tetracycline compounds.

### Example 3

### In Vitro Phototoxicity Assay

3T3 fibroblast cells were harvested and plated at a concentration of 1×10⁵ cells/mL and the plates were incubated overnight at 37° C., in 5% CO₂ and approximately 95% humidity. On the following day the medium was removed from the plates and replaced with Hanks' Balanced Salt Solution (HBSS). Compound dilutions were made in HBSS and added to the plates. For each compound tested, a duplicate plate was prepared that was not exposed to light as a control for compound toxicity. Plates were then incubated in a dark drawer (for controls), or under UV light (meter reading of 1.6-1.8 mW/cm²) for 50 minutes. Cells were then washed with HBSS, fresh medium was added, and plates were incubated overnight as described above. The following day neutral red was added as an indicator of cell viability. The plates were then incubated for an additional 3 hours. Cells were then washed with HBSS and blotted on absorbent paper to remove excess liquid. A solution of 50% EtOH, 10% glacial acetic acid was added and after 20 minutes incubation the plate's absorbance at 535 nm was read using a Wallac Victor 5 spectrophotometer. The phototoxicity reflected the difference between the light-treated and control cultures. Table 3 includes phototoxicity (μM) data for several substituted tetracycline compounds.

TABLE 3

| | | Median MIC's (ug/mL) G+ | | | | | | |
|---|---|---|---|---|---|---|---|---|
| | | | | S. pneu- | Median MIC's (ug/mL) G− | | | |
| | | | | moniae | | E. coli | E. coli | P. aeruginosa |
| | Com- | S. aureus | S. aureus | 157E- | E. coli | ATCC | MG | K201 |
| Structure | pound | RN450 | MRSA5 | Strep | D1 209 | 25922 | 1655 | PAO6609 |
| 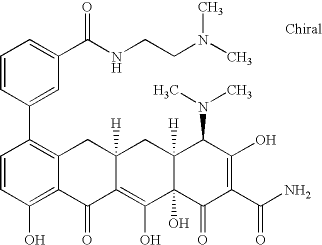 | A | 8 | 64 | 4 | 64 | 64 | 64 | 64 |
| 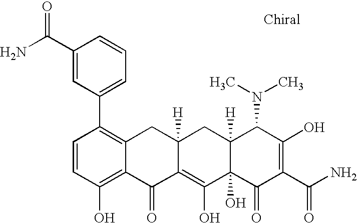 | B | 2 | 64 | 2 | 64 | 64 | 64 | 64 |
| 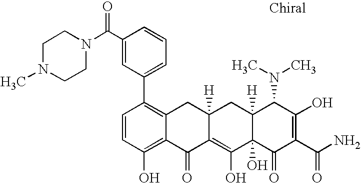 | C | 0.25 | 64 | 0.06 | 64 | 8 | 32 | 64 |
| 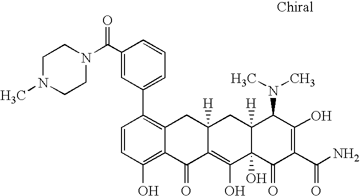 | D | 32 | 64 | 4 | 64 | 64 | 64 | 64 |
| 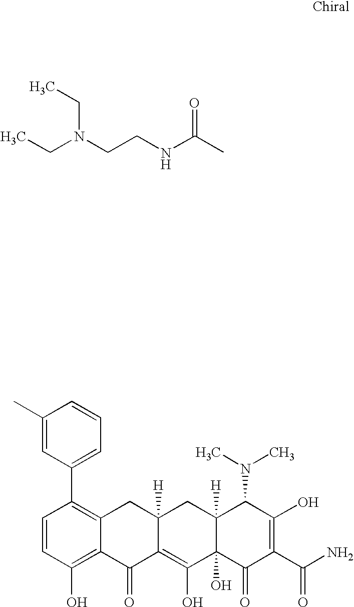 | E | 0.5 | 16 | 0.125 | 64 | 8 | 8 | 64 |
| 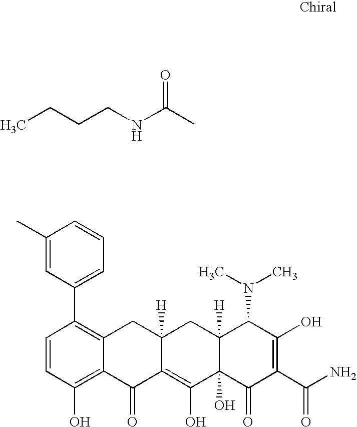 | F | 0.06 | 1 | 0.06 | 64 | 64 | 64 | 64 |
| 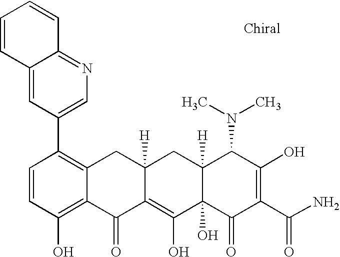 | G | 0.06 | 0.5 | 0.06 | 64 | 4 | 64 | 64 |
| 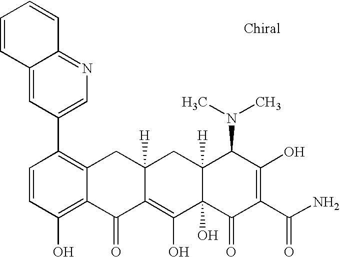 | H | 0.5 | 2 | 0.25 | 64 | 64 | 64 | 64 |
| 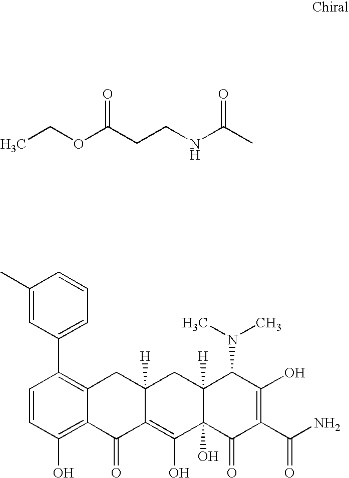 | I | 0.06 | 2 | 3 | 64 | 64 | 64 | 64 |
| 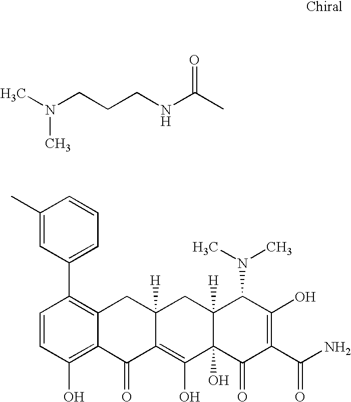 | J | 1 | 64 | 1 | 64 | 32 | 32 | 64 |
| 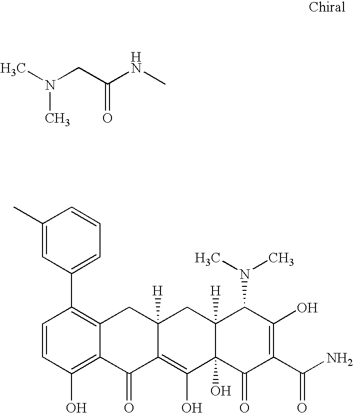 | K | 0.06 | 2 | 0.06 | 2 | 1 | 2 | 64 |
| 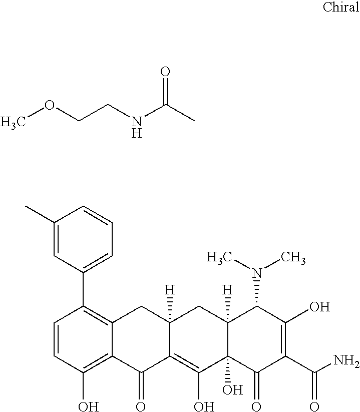 | L | 0.06 | 1 | 0.06 | 64 | 8 | 32 | 64 |
| 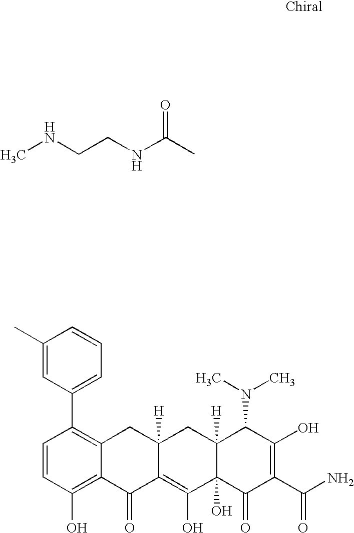 | M | 4 | 64 | 32 | 64 | 64 | 64 | 64 |
| 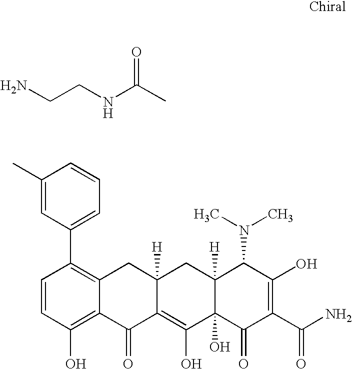 | N | 2 | 64 | 16 | 64 | 64 | 64 | 64 |
| 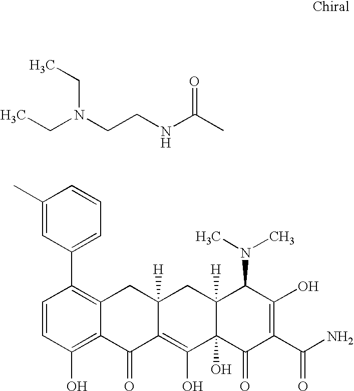 | O | 4 | 64 | 4 | 64 | 64 | 64 | 64 |
| 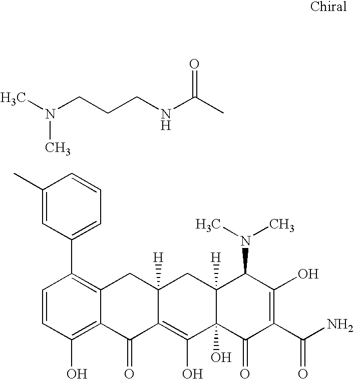 | P | 4 | 64 | 8 | 64 | 64 | 64 | 64 |
| 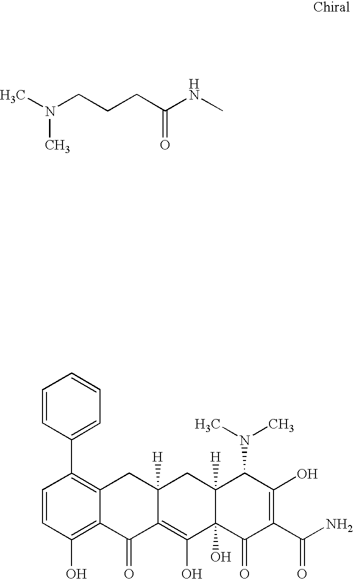 | Q | 2 | 64 | 1 | 64 | 64 | 64 | 64 |
| 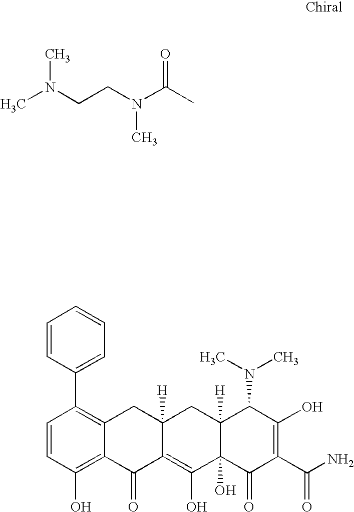 | R | 0.5 | 64 | 0.25 | 64 | 32 | 15 | 64 |
| 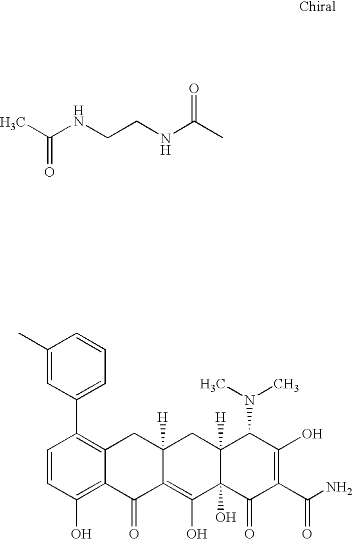 | S | 2 | 32 | 2 | 64 | 64 | 64 | 64 |
| 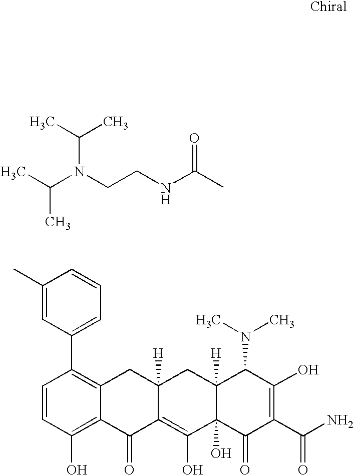 | T | 0.13 | 4 | 0.06 | 8 | 4 | 16 | 64 |
| 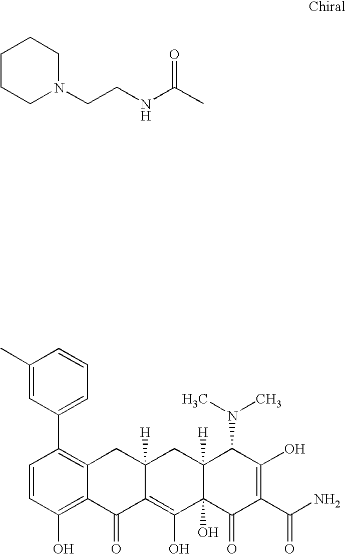 | U | 0.25 | 8 | 0.06 | 8 | 4 | 4 | 64 |
| 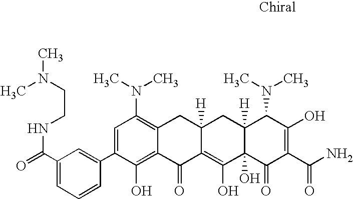 | V | 16 | 64 | 32 | 64 | 64 | 64 | 64 |
| 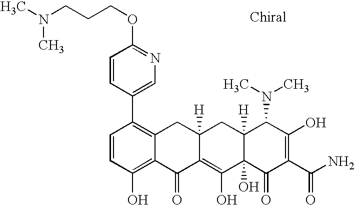 | W | 0.06 | 2 | 0.06 | 2 | 1 | 2 | 64 |
| 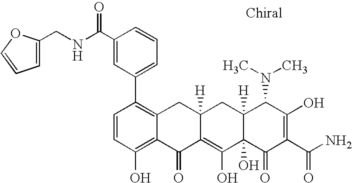 | Y | 0.06 | 1 | 0.06 | 64 | 64 | 64 | 64 |
| 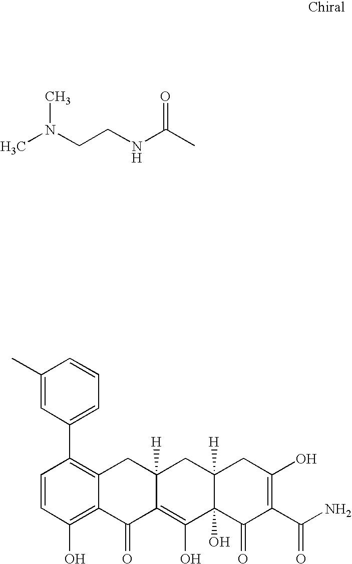 | Z | 64 | 64 | 64 | 64 | 64 | 64 | 64 |
| 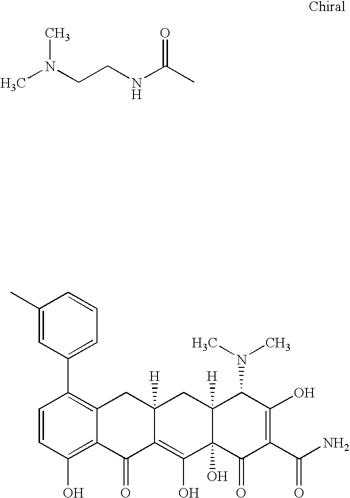 | AB | 0.5 | 32 | 0.25 | 64 | 32 | 16 | 64 |
| 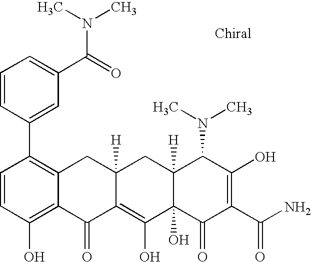 | AC | 0.25 | 16 | — | 64 | — | — | — |
| 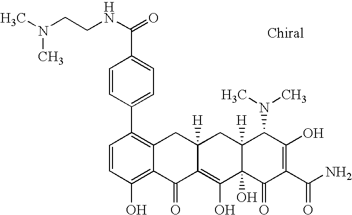 | AD | 0.25 | 16 | 0.13 | 64 | 16 | 8 | 64 |
| 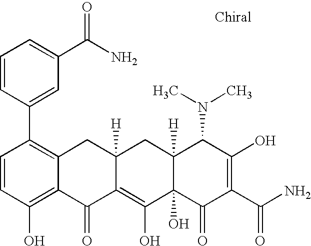 | AE | 0.06 | 4 | 0.06 | 64 | 4 | 4 | 64 |
| 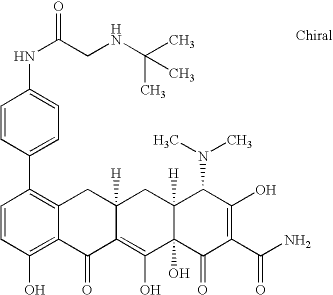 | AF | 0.06 | 4 | 0.06 | 2 | — | — | — |
| 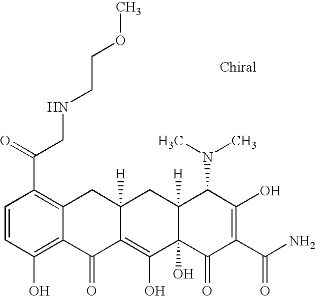 | AG | 4 | 64 | 4 | 64 | 32 | 32 | 64 |
| 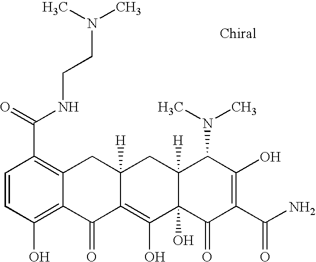 | AH | 16 | 64 | 8 | 64 | 64 | 64 | 64 |
| 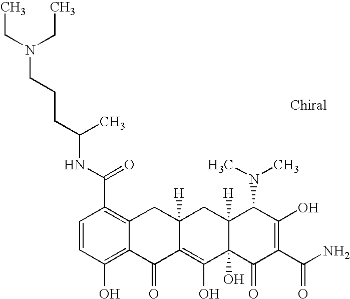 | AI | 64 | 64 | 64 | 64 | 64 | 64 | 64 |
| 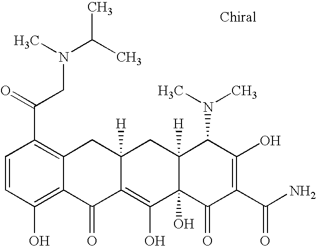 | AJ | 0.06 | 0.5 | 0.06 | 1 | 0.25 | 0.25 | 32 |
| 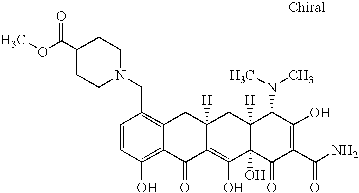 | AK | 0.06 | 2 | 0.06 | 4 | 0.25 | 0.5 | 64 |
| 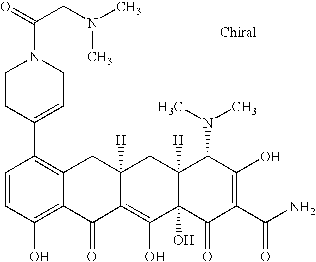 | AL | 0.5 | 32 | 0.5 | 64 | 16 | 16 | 64 |
| 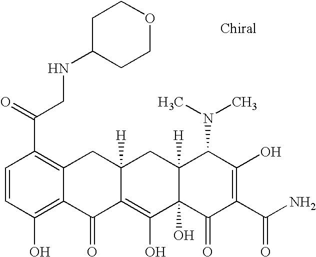 | AM | 4 | 64 | 4 | 64 | 64 | 64 | 64 |
| 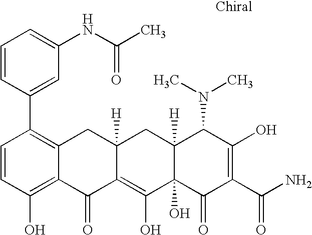 | AO | 0.06 | 4 | 0.06 | 64 | 4 | 32 | 64 |
| 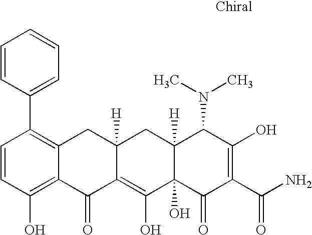 | AQ | 0.06 | 0.25 | 0.06 | 8 | — | — | — |
| 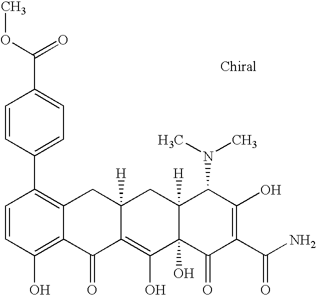 | AR | 0.06 | 0.315 | 0.25 | 64 | — | — | — |
| 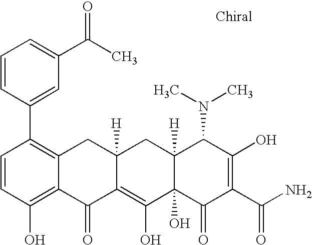 | AS | 0.06 | 0.5 | 0.06 | 64 | — | — | — |
| 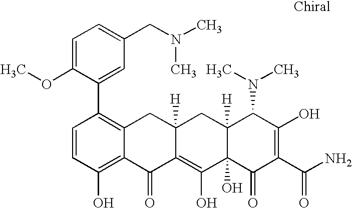 | AT | 0.06 | 0.5 | 0.06 | 1 | 0.5 | 1 | 64 |
| 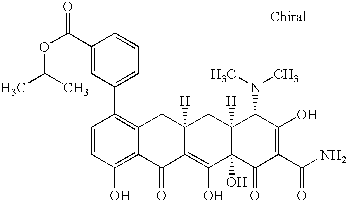 | AU | 0.25 | 1 | 0.5 | 64 | — | — | — |
| 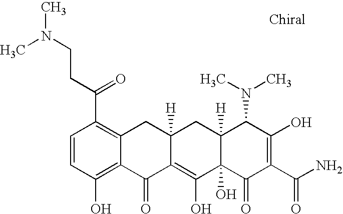 | AV | 0.06 | 0.5 | 0.5 | 64 | 8 | 16 | 64 |
| 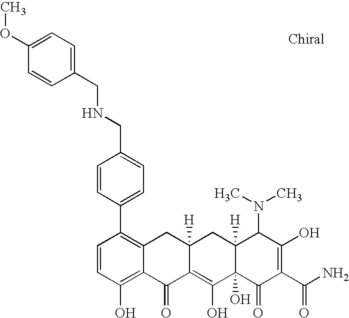 | AW | 0.06 | 1 | 0.06 | 1 | — | — | — |
| 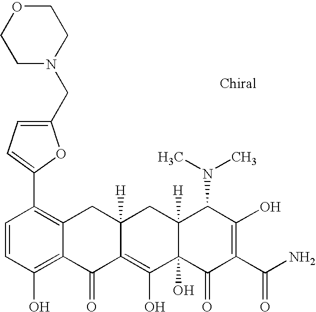 | AX | 0.06 | 4 | 0.06 | 8 | — | — | — |
| 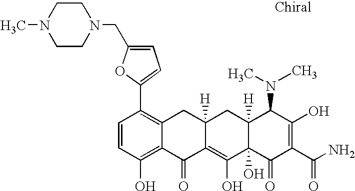 | AY | 0.5 | 32 | 0.25 | 64 | — | — | — |
| 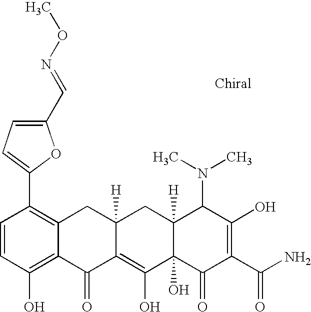 | AZ | 0.13 | 1 | 0.06 | 64 | — | — | — |
| 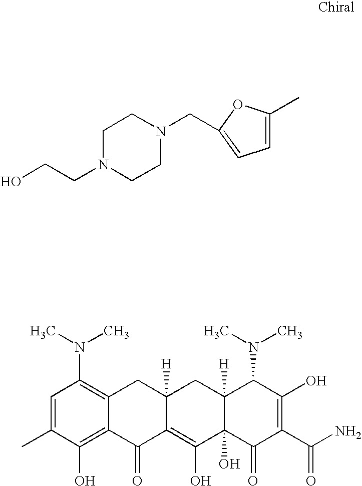 | BA | 1 | 64 | 2 | 64 | — | — | — |
| 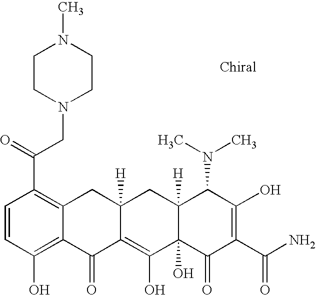 | BB | 2 | 16 | 1 | 64 | 32 | 8 | 64 |
| 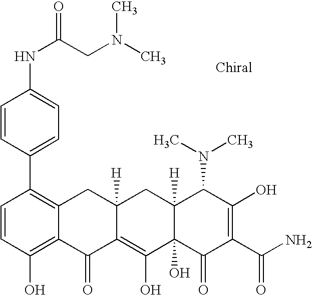 | BC | 0.06 | 2 | 0.06 | 1 | 1 | 2 | 64 |
| 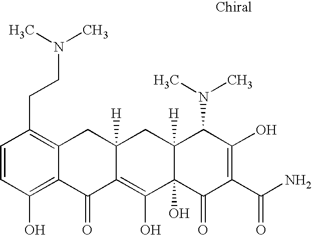 | BD | 0.5 | 32 | 0.13 | 64 | 4 | 4 | 64 |
| 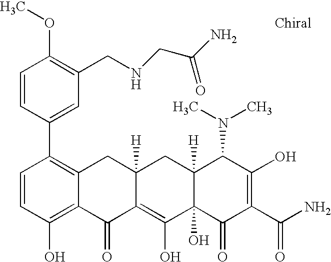 | BE | 1 | 32 | 0.13 | 64 | 64 | 16 | 64 |
| 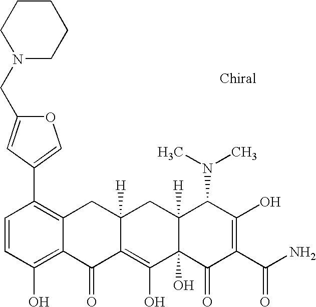 | BF | 0.06 | 1 | 0.06 | 0.5 | 0.25 | 1 | 64 |
| 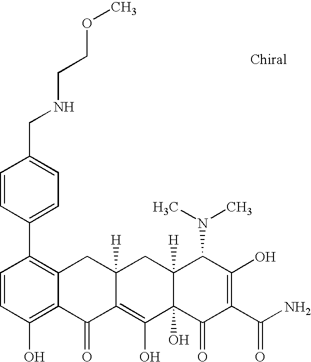 | BG | 0.06 | 1 | 0.06 | 0.5 | 0.25 | 0.5 | 64 |
| 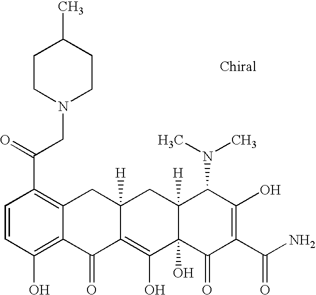 | BH | 0.06 | 0.06 | 0.06 | 0.25 | 0.06 | 0.06 | 32 |
| 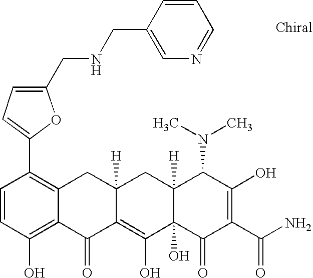 | BI | 0.06 | 8 | 0.06 | 2 | 1 | 4 | 64 |
| 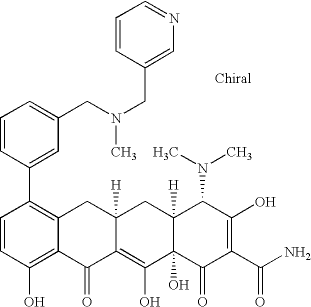 | BJ | 0.25 | 2 | 0.06 | 32 | 16 | 64 | 64 |
| 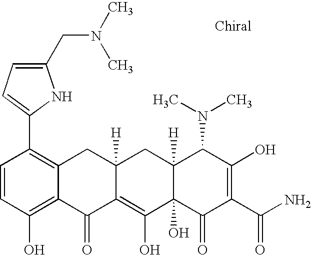 | BK | 0.25 | 4 | 0.13 | 64 | 4 | 4 | 32 |
| 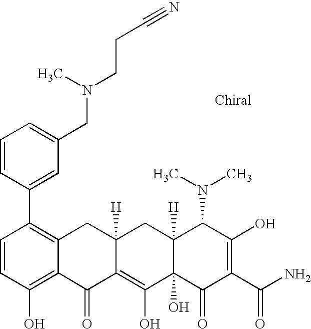 | BL | 0.06 | 2 | 0.06 | 16 | 4 | 16 | 64 |
| 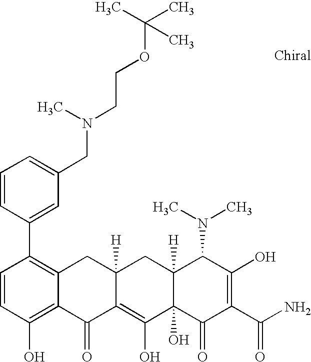 | BM | 0.13 | 2 | 0.06 | 4 | 4 | 8 | 64 |
| 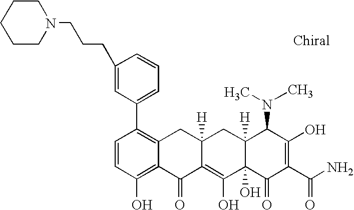 | BN | 4 | 32 | 0.25 | 32 | 16 | 32 | 64 |
| 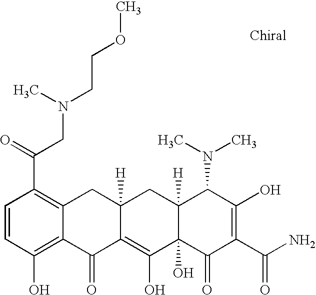 | BO | 0.5 | 4 | 0.25 | 64 | 2 | 2 | 64 |
| 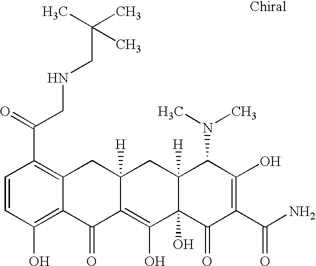 | BP | 2 | 16 | 2 | 64 | 32 | 32 | 64 |
| 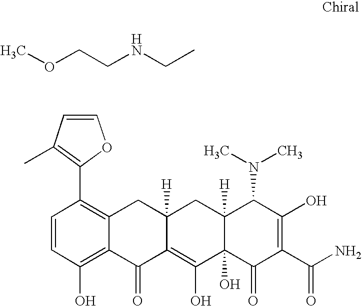 | BQ | 0.06 | 4 | 0.06 | 64 | 1 | 1 | 64 |
| 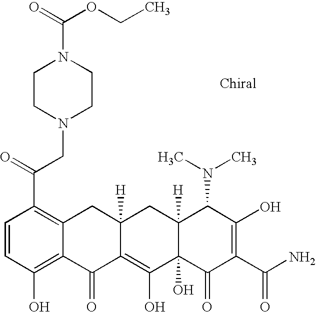 | BR | 0.06 | 0.5 | 0.06 | 64 | 8 | 8 | 64 |
| 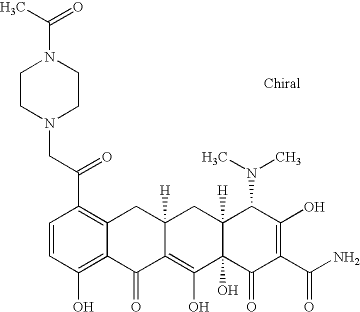 | BS | 1 | 8 | 1 | 64 | 64 | 32 | 64 |
| 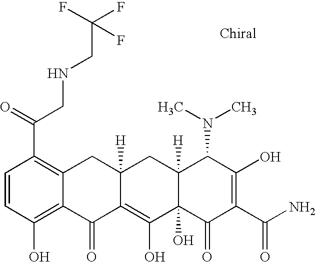 | BT | 0.06 | 2 | 0.06 | 64 | 2 | 8 | 64 |
| 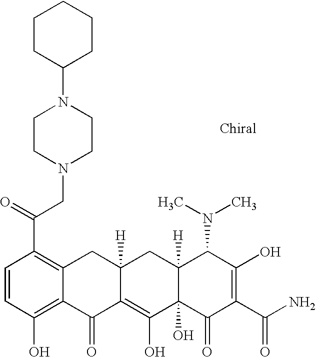 | BU | 0.13 | 1 | 0.06 | 2 | 1 | 0.5 | 64 |
| 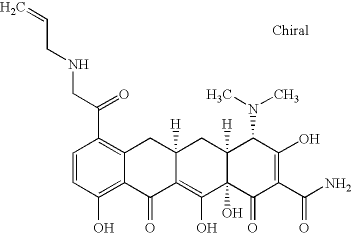 | BV | 4 | 32 | 1 | 64 | 32 | 16 | 64 |
| 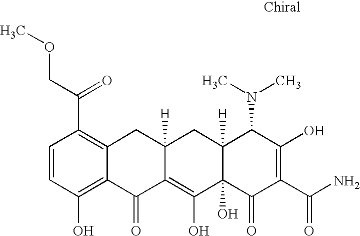 | BW | 0.06 | 1 | 0.06 | 64 | 0.5 | 0.5 | 32 |
| 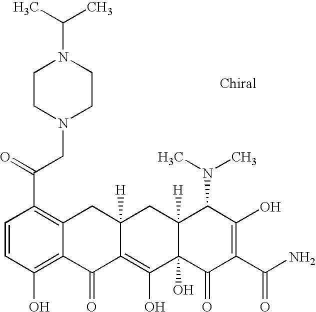 | BX | 1 | 4 | 0.5 | 64 | 8 | 8 | 64 |
| 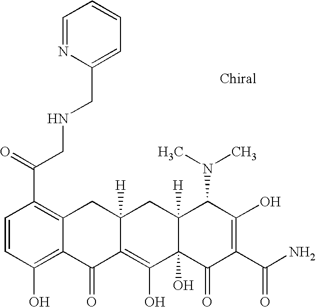 | BY | 2 | 32 | 4 | 64 | 64 | 64 | 64 |
| 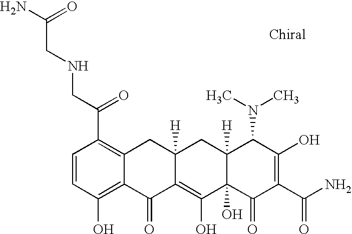 | BZ | 4 | 64 | 4 | 64 | 64 | 64 | 64 |
| 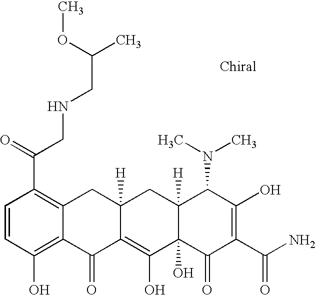 | CA | 4 | 32 | 2 | 64 | 32 | 32 | 64 |
| 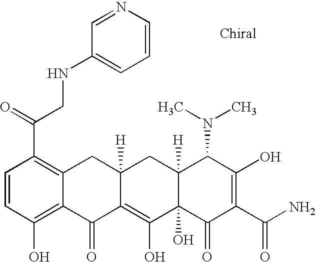 | CB | 16 | 64 | 32 | 64 | 64 | 64 | 64 |
| 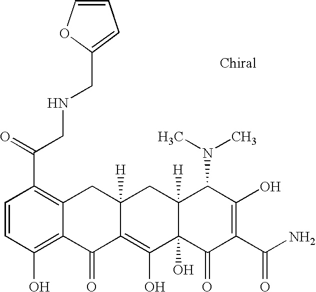 | CC | 1 | 16 | 2 | 64 | 32 | 32 | 64 |
| 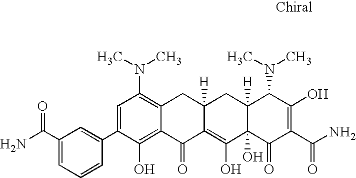 | CD | 8 | 32 | 16 | 64 | 64 | 64 | 64 |
| 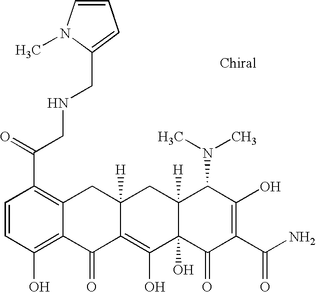 | CE | 0.25 | 4 | 0.5 | 64 | 4 | 4 | 64 |
| 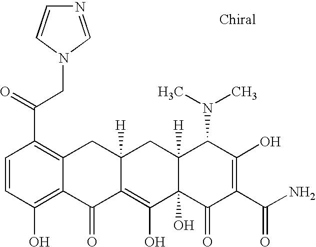 | CF | 4 | 64 | 4 | 64 | 64 | 64 | 64 |
| 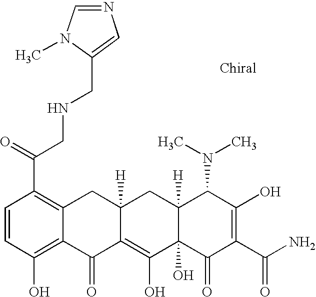 | CG | 0.13 | 64 | 1 | 64 | 16 | 16 | 64 |
| 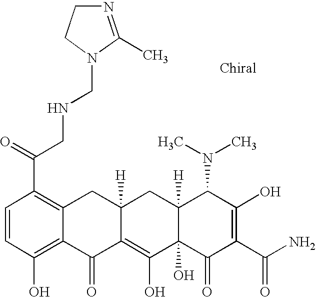 | CH | 16 | 64 | 64 | 64 | 64 | 64 | 64 |
| 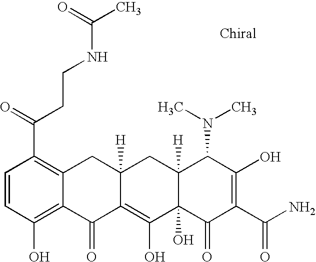 | CI | 2 | 64 | 2 | 64 | 64 | 64 | 64 |
| 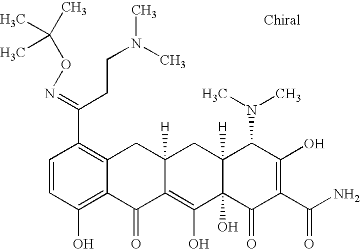 | CJ | 0.06 | 2 | 0.06 | 1 | 0.5 | 2 | 64 |
| 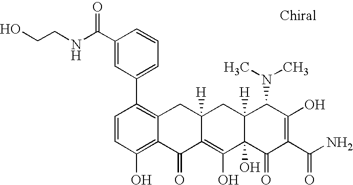 | CK | 0.125 | 8 | 0.125 | 64 | 64 | 64 | 64 |
| 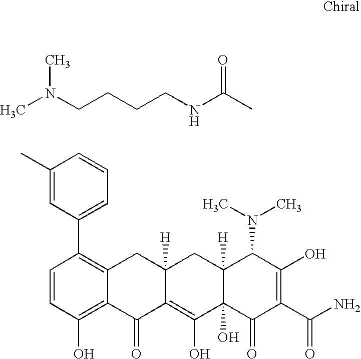 | CL | 4 | 64 | 4 | 64 | 64 | 64 | 64 |
| 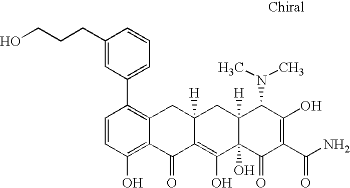 | CM | 0.0625 | 0.5 | 0.06 | 64 | 64 | 64 | 64 |
| 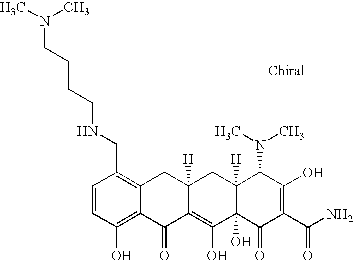 | CN | 16 | 64 | 32 | 64 | 32 | 32 | 64 |
| 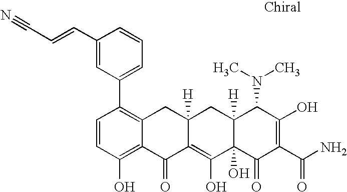 | CO | 0.0625 | 0.5 | 0.06 | 64 | 64 | 64 | 64 |
| 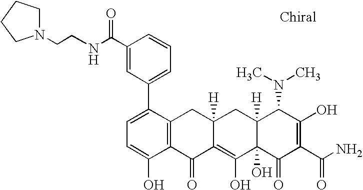 | CR | 1 | 16 | 0.25 | 64 | 64 | 16 | 64 |
| 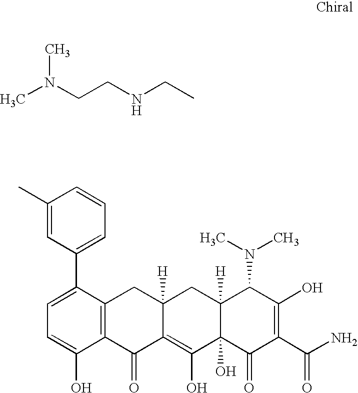 | CS | 0.06 | 8 | 0.06 | 64 | 4 | 2 | 64 |
| 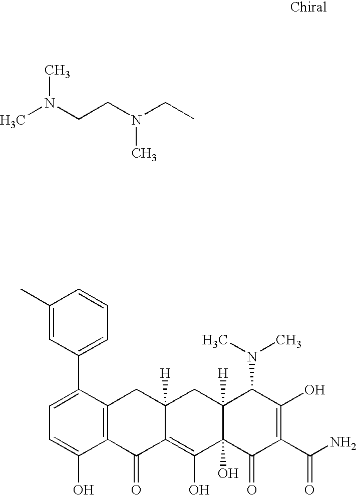 | CT | 0.0625 | 4 | 0.06 | 4 | 2 | 2 | 64 |
| 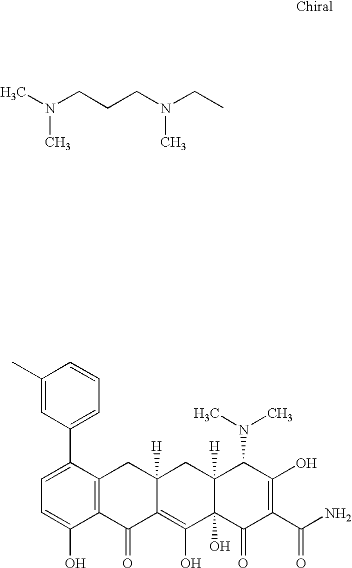 | CU | 0.5 | 16 | 0.125 | 32 | 8 | 8 | 32 |
| 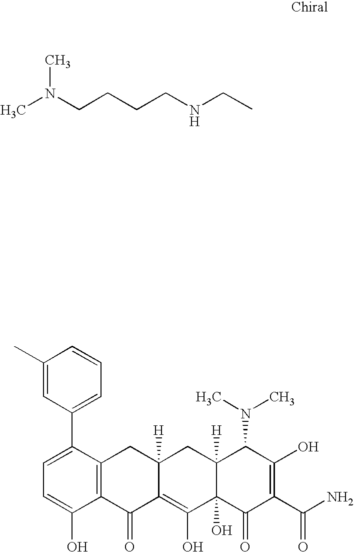 | CV | 2 | 32 | 4 | 64 | 64 | 64 | 64 |
| 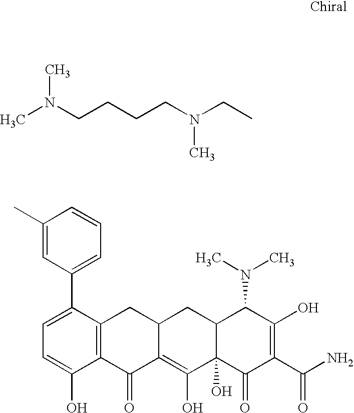 | CW | 0.06 | 8 | 0.06 | 4 | 2 | 2 | 64 |
| 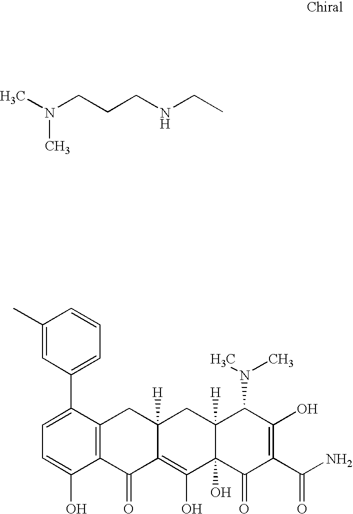 | CX | 0.5 | 8 | 0.25 | 32 | 16 | 16 | 64 |
| 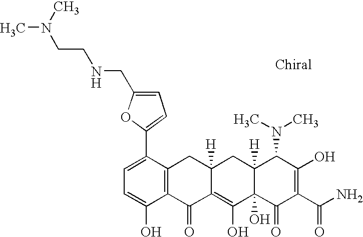 | CY | 0.25 | 64 | 0.06 | 64 | 4 | 2 | 64 |
| 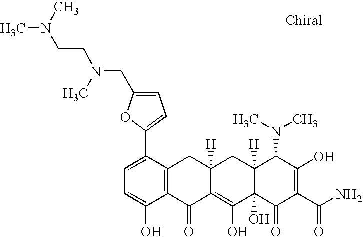 | CZ | 0.06 | 16 | 0.06 | 16 | 4 | 4 | 64 |
| 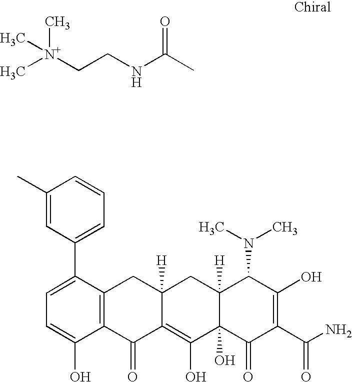 | DA | 4 | 64 | 32 | 64 | 64 | 64 | 64 |
| 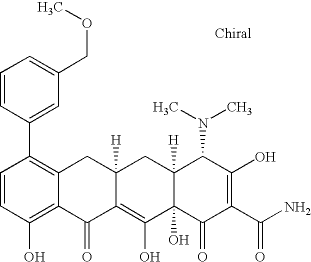 | DB | 0.06 | 0.5 | 0.06 | 64 | — | — | — |
| 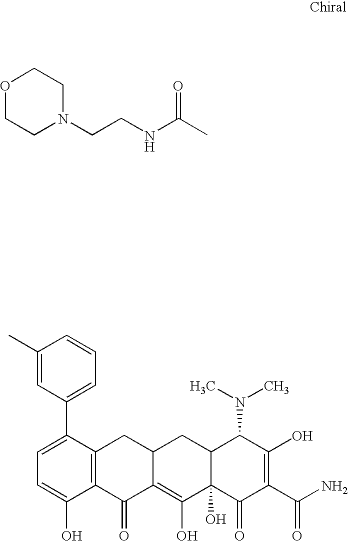 | DC | 0.125 | 4 | 0.125 | 64 | 16 | 8 | 64 |
| 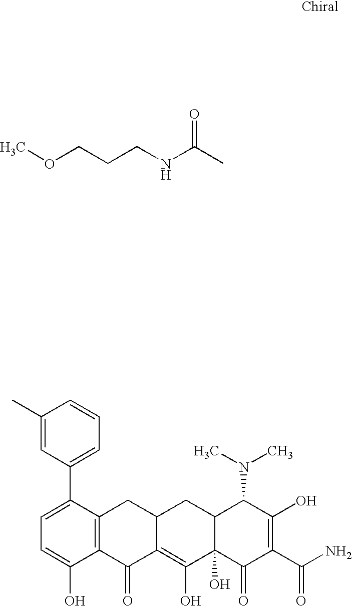 | DD | 0.0625 | 2 | 0.06 | 64 | 64 | 64 | 64 |
| 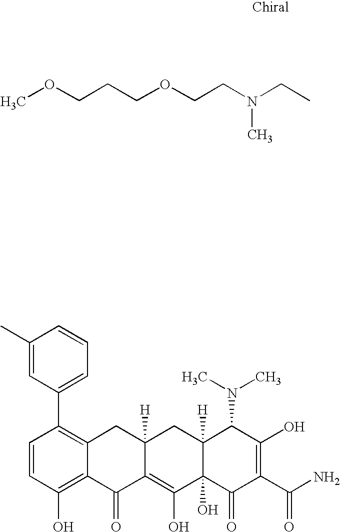 | DE | 0.06 | 2 | 0.06 | 2 | 2 | 8 | 64 |
| 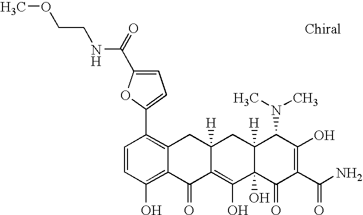 | DF | 0.0625 | 16 | 0.06 | 64 | 16 | 32 | 64 |
| 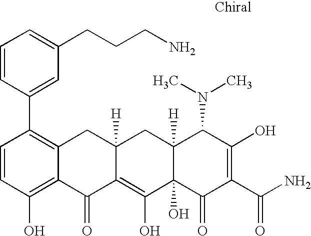 | DG | 0.5 | 64 | 0.125 | 32 | 32 | 32 | 64 |
| 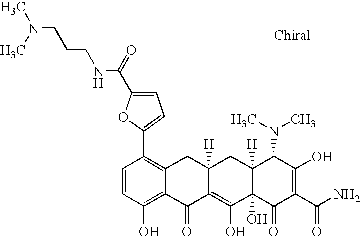 | DH | 4 | 64 | 2 | 64 | 64 | 64 | 64 |
| 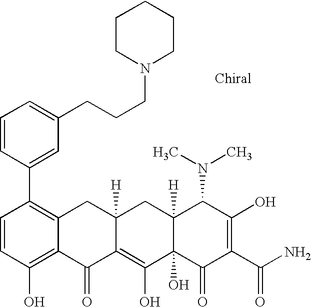 | DI | 0.0625 | 2 | 0.06 | 2 | 1 | 4 | 64 |
| 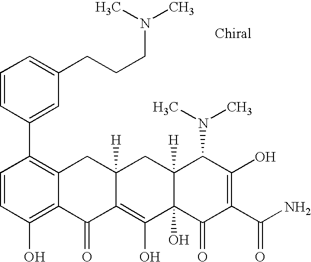 | DJ | 0.0625 | 4 | 0.06 | 2 | 2 | 8 | 64 |
| 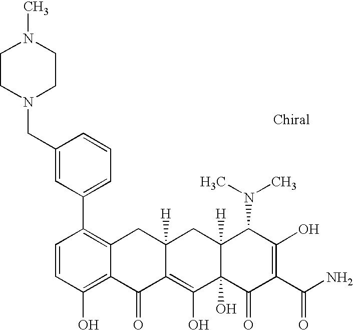 | DK | 0.0625 | 4 | 0.06 | 2 | 1 | 4 | 64 |

| | |
|---|---|
| Cytotox. (ug/mL) | Photo Toxicity |

| | | | | | |
|---|---|---|---|---|---|
| Structure | Compound | COS-1 | CHO-K1 | Dark Tox50 (uM) | UV Tox50 (uM) |

| | | | | | |
|---|---|---|---|---|---|
| 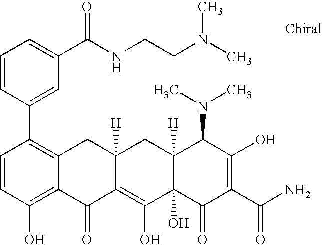 | A | >200 | >200 | M: >200 | M: >200 |
| 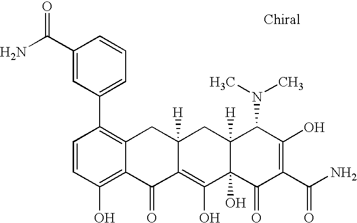 | B | >15.66 | >15.66 | >156.64 | >156.64 |
| 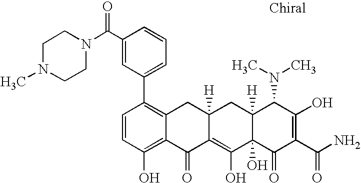 | C | >137.76 | >137.76 | >137.76 | >137.76 |
| 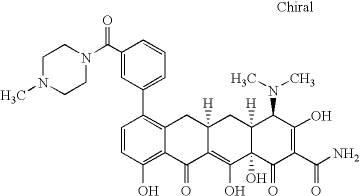 | D | >130.51 | >130.51 | >130.51 | >130.51 |
| 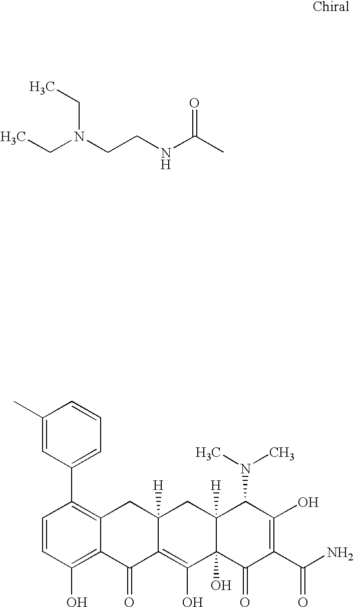 | E | >138.88 | >138.88 | >138.88 | >138.88 |
| 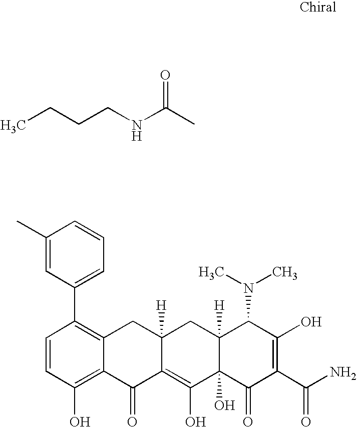 | F | 15.17 | >15.17 | >151.73 | >151.73 |
| 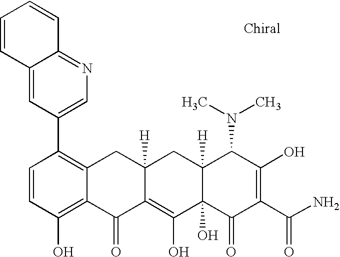 | G | 15.94 | 14.75 | >159.48 | 19.29 |
| 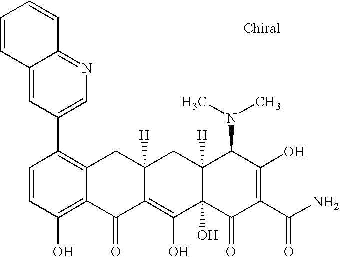 | H | >15.94 | >15.94 | >159.48 | >159.48 |
| 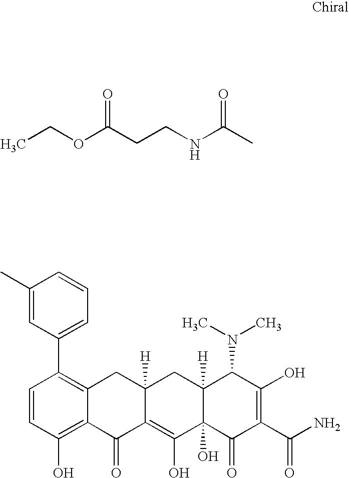 | I | >14.17 | >14.17 | >141.76 | >141.76 |
| 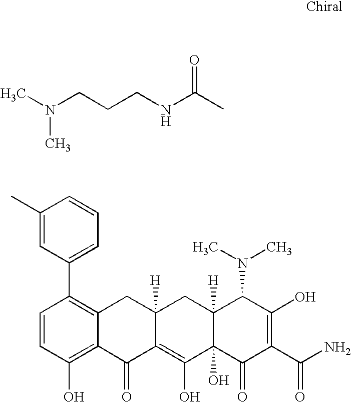 | J | >40.34 | >40.34 | >130.13 | >130.13 |
| 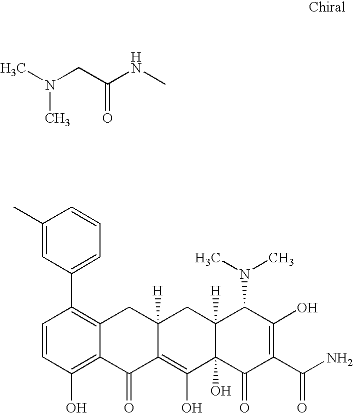 | K | >12.8 | >12.8 | >128.09 | >128.09 |
| 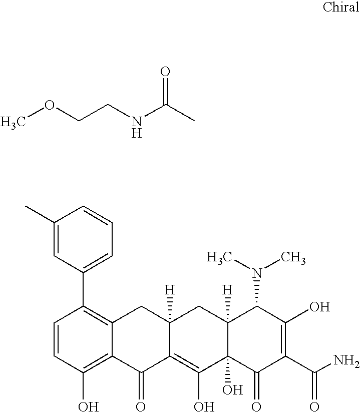 | L | >15.12 | >15.12 | >151.25 | >151.25 |
| 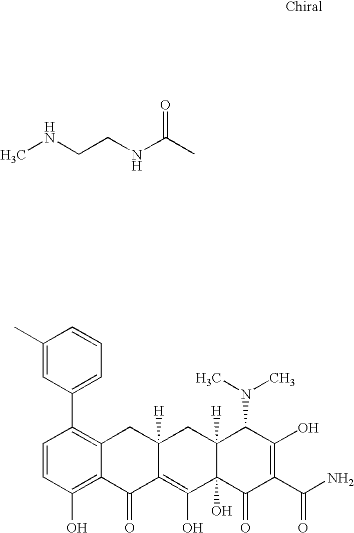 | M | >143.16 | >143.16 | >143.16 | >143.16 |
| 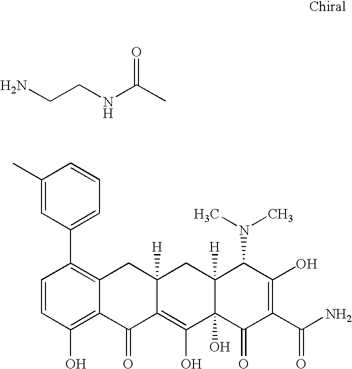 | N | >46.77 | >46.77 | >150.87 | 95.14 |
| 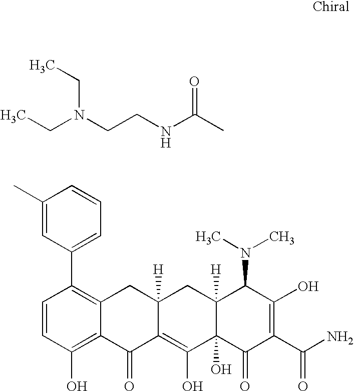 | O | >134.63 | >134.63 | >134.63 | >134.63 |
| 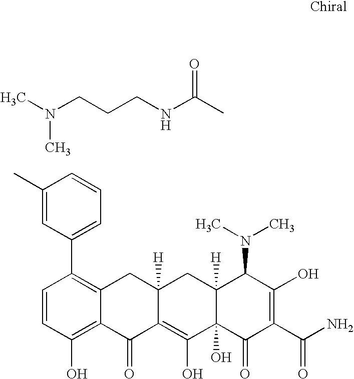 | P | >122.9 | >122.9 | >122.9 | >122.9 |
| 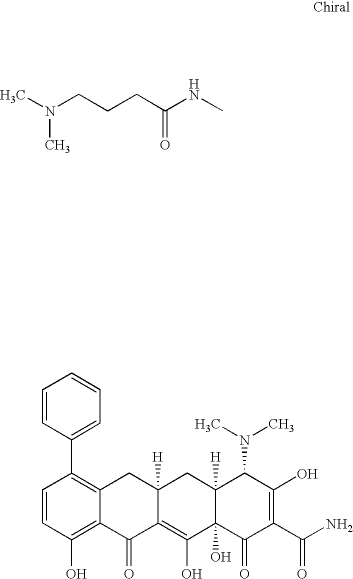 | Q | >137.36 | >137.36 | >137.36 | >137.36 |
| 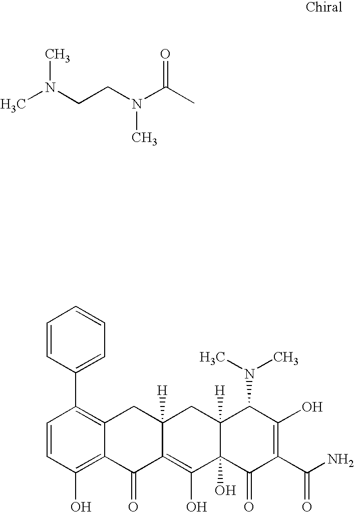 | R | >137.36 | >137.36 | >137.36 | >137.36 |
| 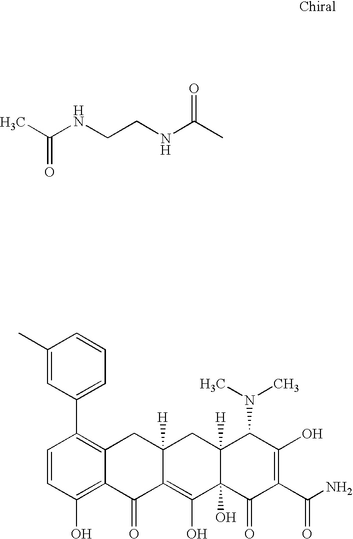 | S | >12.97 | >12.97 | >129.75 | >129.75 |
| 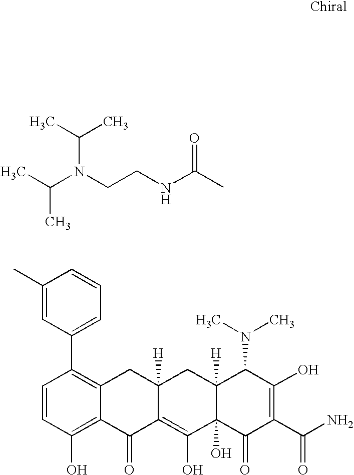 | T | >41.4 | 36.17 | >133.57 | >133.57 |
| 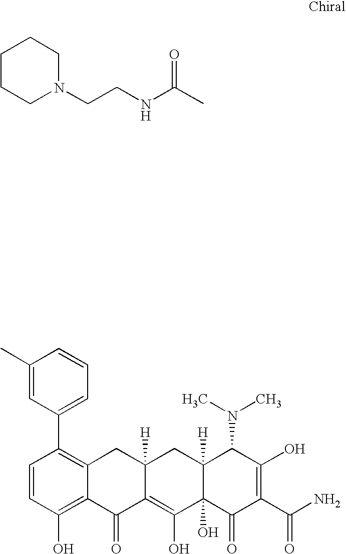 | U | >41.03 | >41.03 | >132.37 | >132.37 |
| 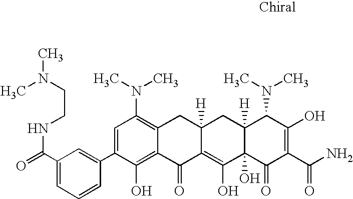 | V | >125.47 | >125.47 | >125.47 | >125.47 |
| 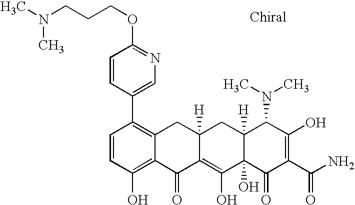 | W | >128.2 | >128.2 | >128.2 | >128.2 |
| 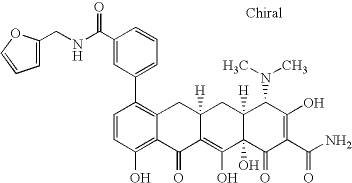 | Y | >14.61 | >14.61 | >146.13 | >146.13 |
| 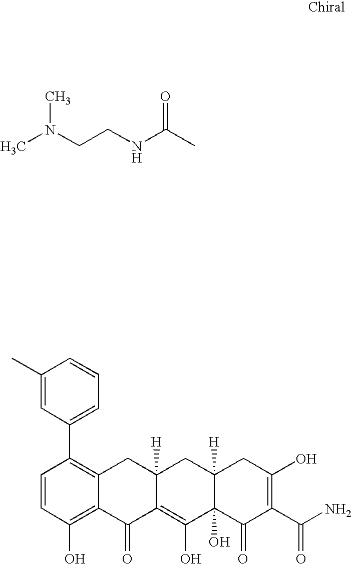 | Z | >15.88 | >15.88 | >158.85 | >158.85 |
| 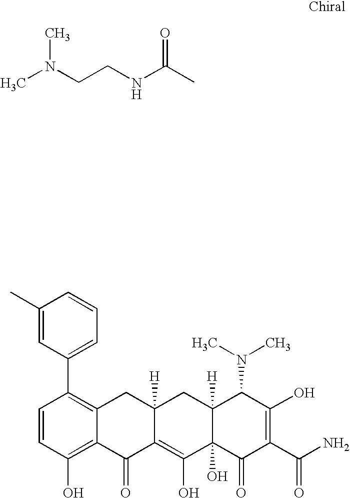 | AB | >200 | >200 | >200 | >200 |
| 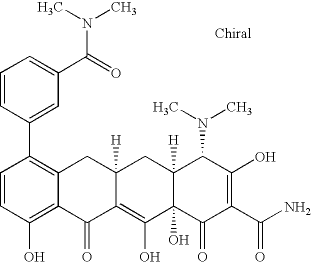 | AC | >37.62 | >9.4 | — | — |
| 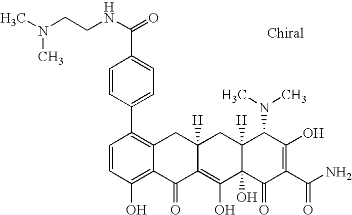 | AD | >43.46 | >43.46 | >140.2 | 135.17 |
| 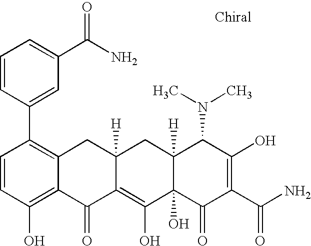 | AE | >15.66 | >15.66 | >156.64 | >156.64 |
| 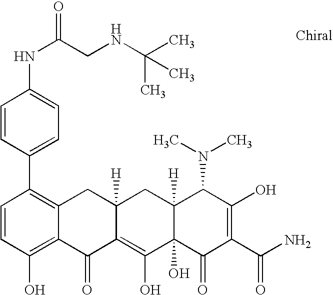 | AF | >13.73 | >13.73 | >137.36 | >137.36 |
| 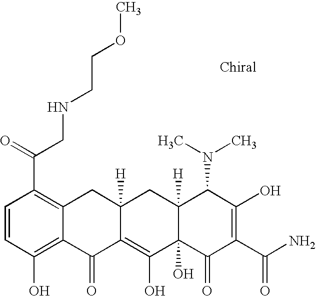 | AG | >157.68 | >157.68 | >157.68 | >157.68 |
| 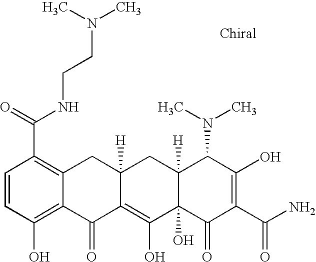 | AH | >125.56 | >125.56 | >125.56 | >125.56 |
| 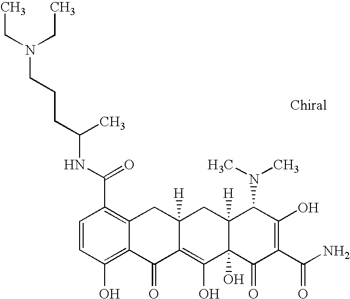 | AI | >108.86 | >108.86 | >108.86 | >108.86 |
| 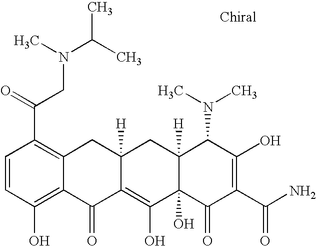 | AJ | >149.87 | >149.87 | >149.87 | >149.87 |
| 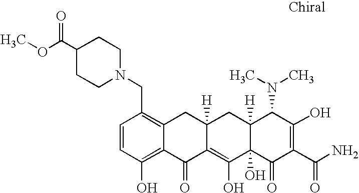 | AK | >140.07 | >140.07 | >140.07 | >140.07 |
| 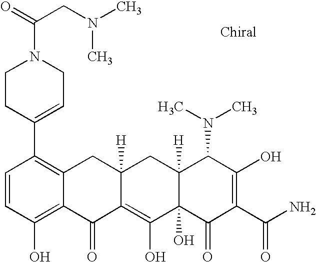 | AL | >145.35 | >145.35 | >145.35 | >145.35 |
| 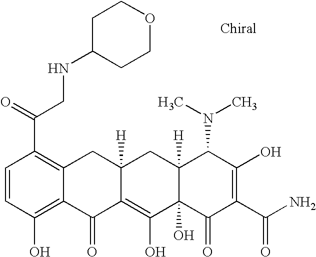 | AM | >143.19 | >143.19 | >143.19 | >143.19 |
| 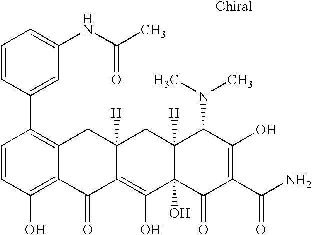 | AO | >16.26 | >16.26 | >162.66 | >162.66 |
| 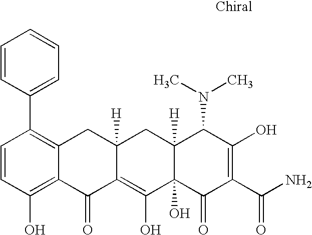 | AQ | 6.06 | 6.91 | — | — |
| 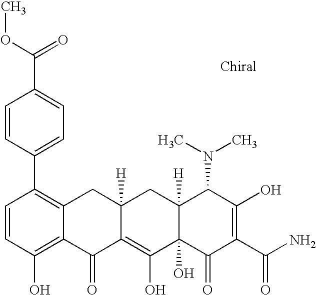 | AR | 4.8 | <2.53 | — | — |
| 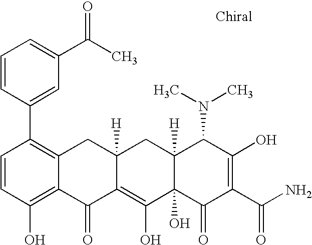 | AS | — | — | — | — |
| 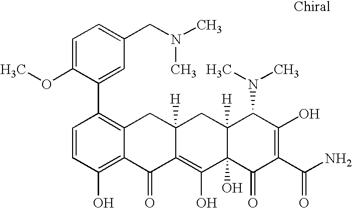 | AT | >146.03 | >146.03 | >146.03 | >146.03 |
| 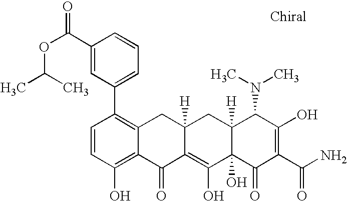 | AU | 4.56 | 6.12 | — | — |
| 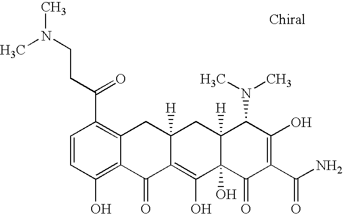 | AV | >153.46 | >153.46 | >153.46 | >153.46 |
| 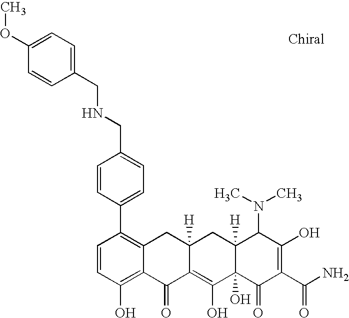 | AW | <1.97 | M: 13.495 | — | — |
| 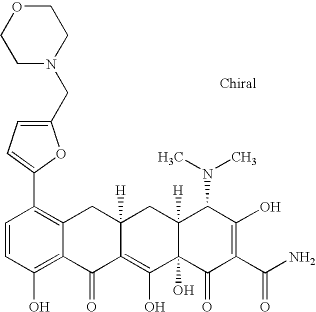 | AX | 27.14 | >32.56 | — | — |
| 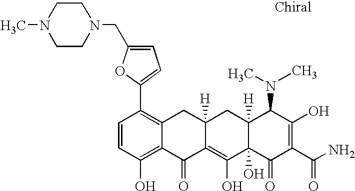 | AY | >32.05 | >32.05 | — | — |
| 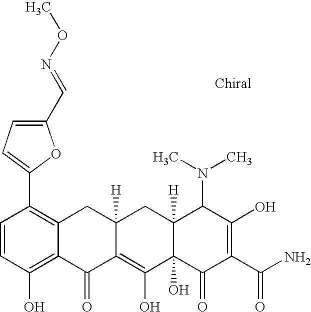 | AZ | <2.44 | 3.04 | >156.79 | >156.79 |
| 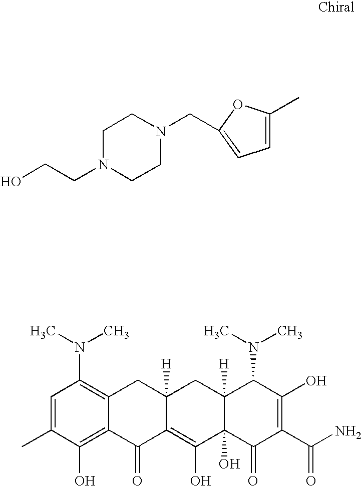 | BA | >110.89 | >110.89 | — | — |
| 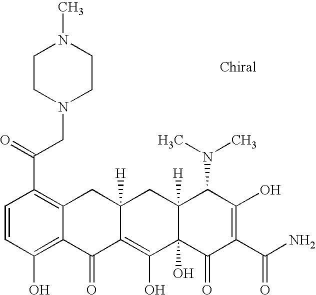 | BB | >135.54 | >135.54 | >135.54 | >135.54 |
| 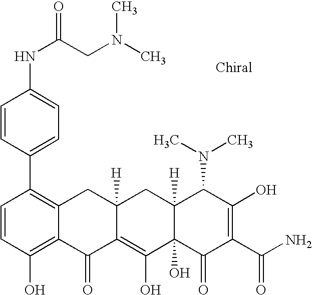 | BC | >13.56 | >13.56 | >135.63 | >135.63 |
| 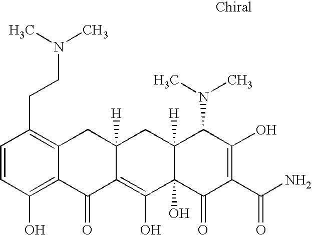 | BD | >120.91 | >120.91 | M: >120.91 | M: >120.91 |
| 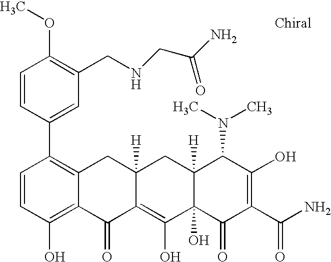 | BE | >10.78 | >10.78 | >107.82 | >107.82 |
| 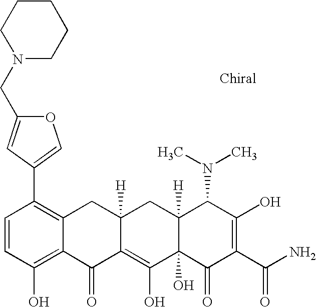 | BF | >42.88 | >42.88 | >138.34 | >138.34 |
| 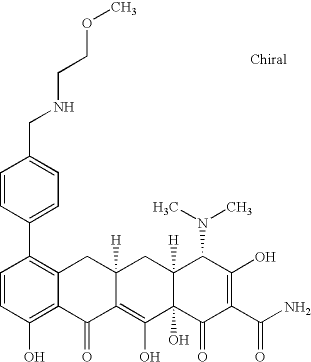 | BG | M: >95.645 | R: >45.26 − 124.56 | >146.03 | >146.03 |
| 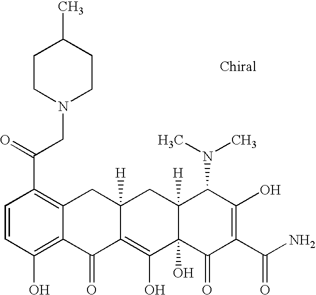 | BH | >135.66 | >135.66 | >135.66 | >135.66 |
| 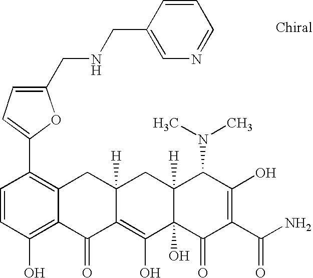 | BI | >39.29 | M: >25.98 | >126.75 | >126.75 |
| 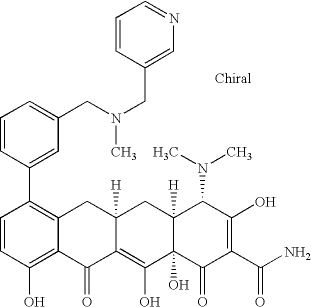 | BJ | >12.26 | M: >25.13 | >122.6 | >122.6 |
| 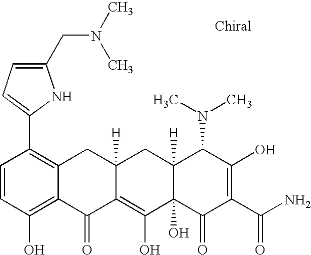 | BK | >147.66 | >147.66 | >147.66 | >147.66 |
| 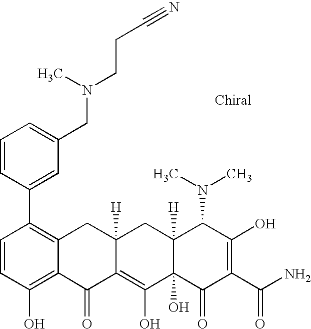 | BL | >13.64 | >13.64 | >136.45 | >136.45 |
| 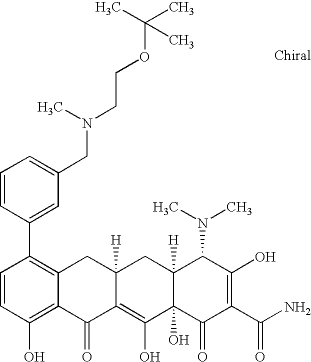 | BM | 22.82 | 21.64 | >104.43 | 5.93 |
| 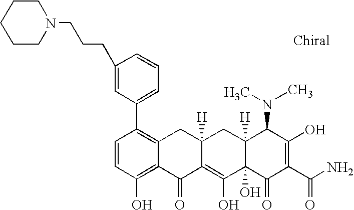 | BN | >63 | >63 | >200 | 33.41189125 |
| 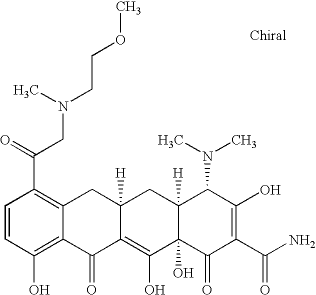 | BO | >154.09 | >154.09 | >154.09 | >154.09 |
| 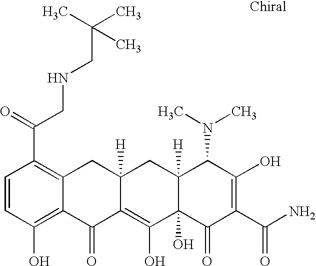 | BP | >42.87 | >42.87 | >138.32 | >138.32 |
| 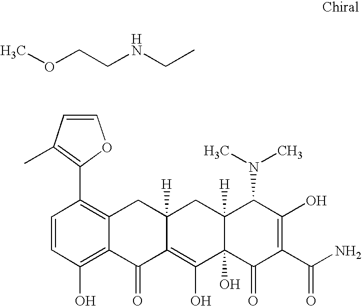 | BQ | >140.51 | >140.51 | >140.51 | >140.51 |
| 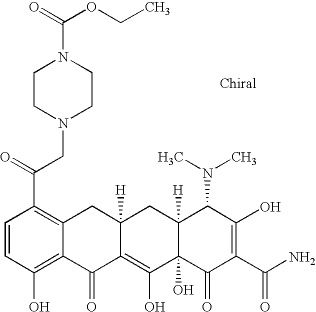 | BR | >40.69 | >40.69 | >131.28 | >131.28 |
| 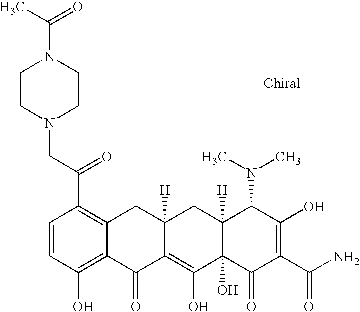 | BS | >144.92 | >144.92 | >144.92 | >144.92 |
| 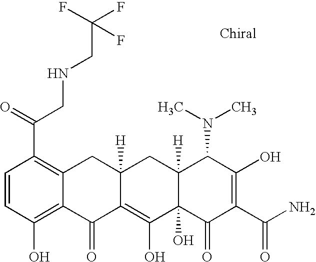 | BT | >47.01 | >47.01 | >151.65 | >151.65 |
| 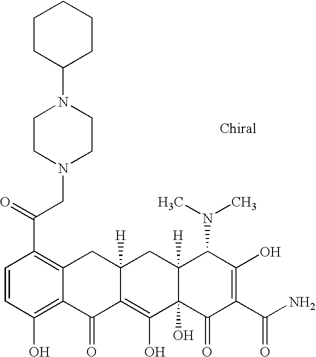 | BU | >122.93 | >122.93 | >122.93 | >122.93 |
| 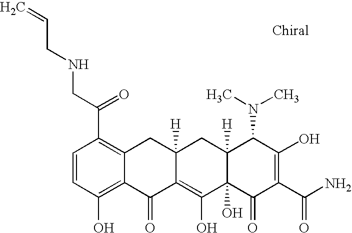 | BV | >121.69 | 27.61 | >121.69 | >121.69 |
| 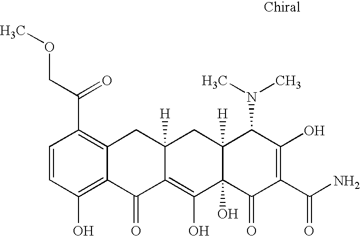 | BW | >53.35 | >53.35 | >172.1 | >172.1 |
| 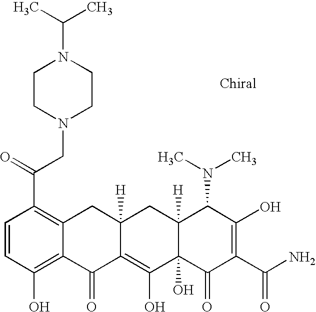 | BX | >130.05 | >130.05 | >130.05 | >130.05 |
| 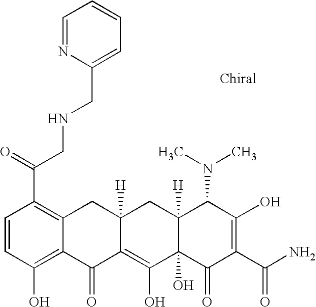 | BY | >13.37 | >13.37 | >133.75 | >133.75 |
| 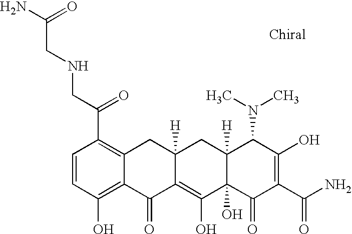 | BZ | >118.96 | >118.96 | >118.96 | >118.96 |
| 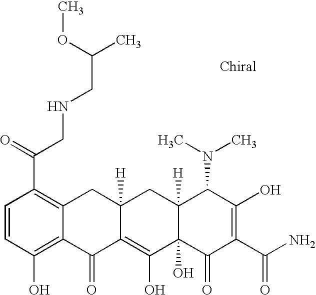 | CA | >145.98 | >145.98 | >145.98 | >145.98 |
| 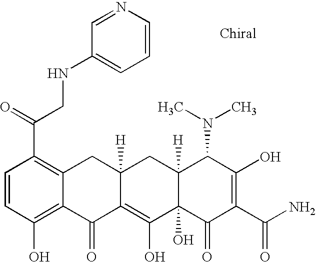 | CB | >152.86 | 110.06 | >152.86 | >152.86 |
| 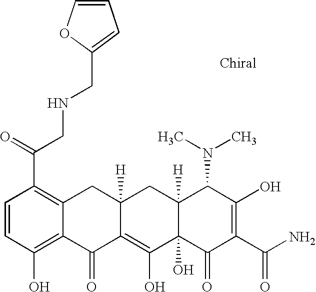 | CC | >44.67 | >44.67 | >144.12 | >144.12 |
| 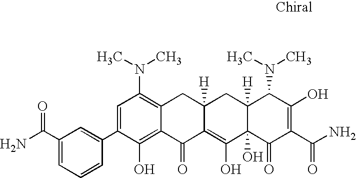 | CD | >45.34 | >45.34 | >146.26 | >146.26 |
| 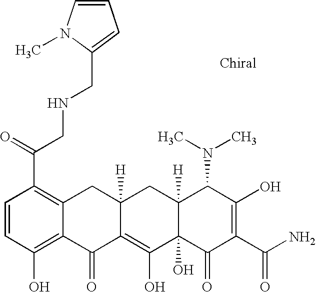 | CE | >41.39 | >41.39 | >133.53 | >133.53 |
| 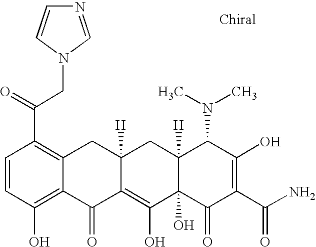 | CF | >49.91 | >49.91 | >161.01 | >161.01 |
| 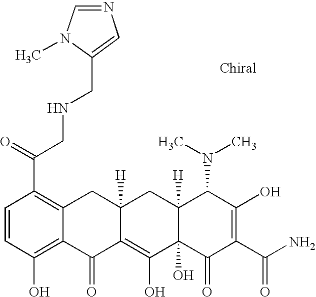 | CG | >45.77 | >45.77 | >147.67 | >147.67 |
| 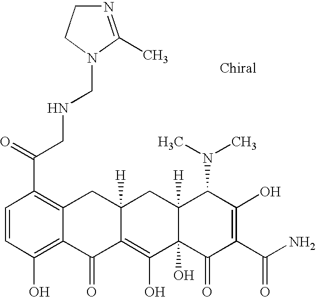 | CH | >131.18 | >131.18 | >131.18 | >131.18 |
| 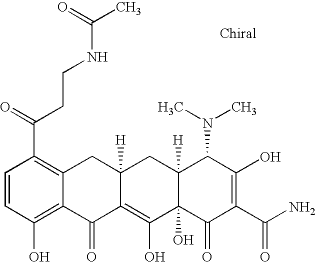 | CI | >159.57 | >159.57 | >159.57 | >159.57 |
| 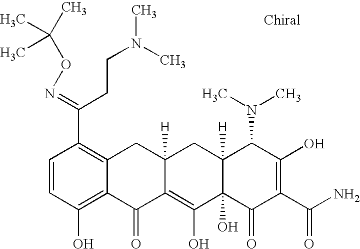 | CJ | 82.24 | 34.64 | >136.86 | 62.89 |
| 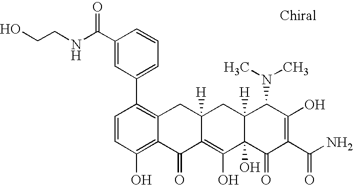 | CK | >14.65 | >14.65 | >146.58 | >146.56 |
| 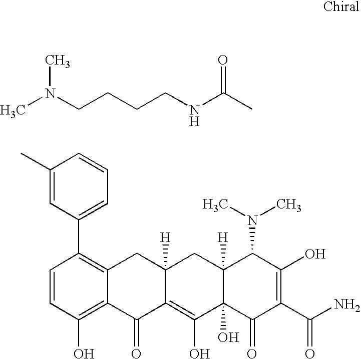 | CL | >41.73 | >41.73 | >134.63 | >134.63 |
| 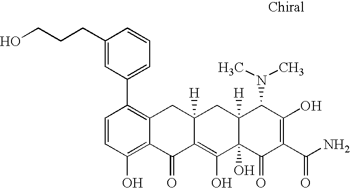 | CM | 15.09 | 14.13 | >167.5 | >167.5 |
| 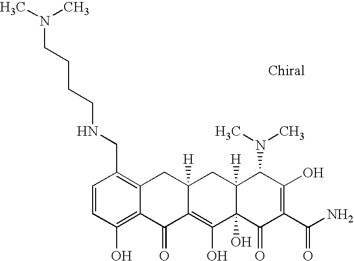 | CN | >138.03 | >138.03 | >138.03 | >138.03 |
| 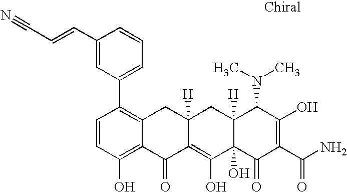 | CO | 7.77 | 9.41 | >164.35 | >164.35 |
| 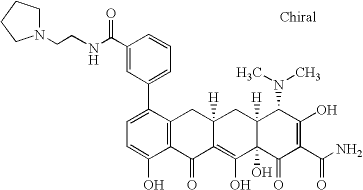 | CR | >39.56 | >39.65 | >127.91 | >127.91 |
| 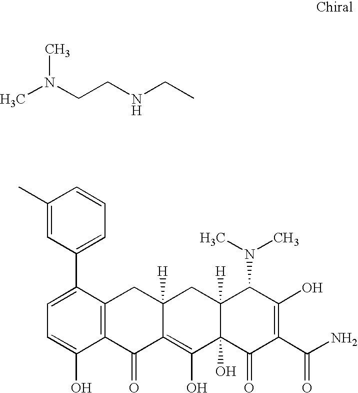 | CS | >139.98 | >139.98 | >139.98 | >139.98 |
| 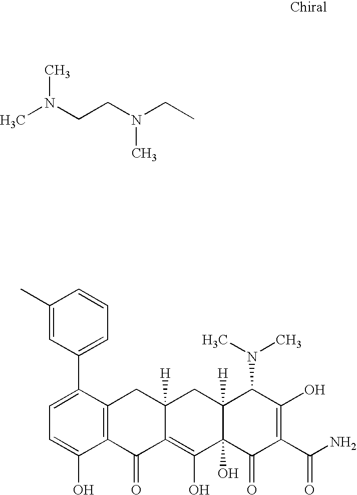 | CT | >126.03 | >126.03 | >126.03 | >126.03 |
| 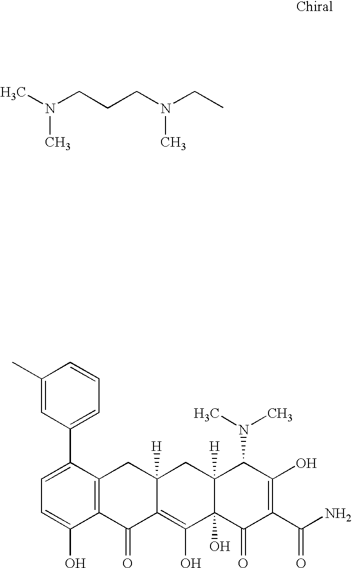 | CU | >123.6 | >123.6 | >123.6 | >123.6 |
| 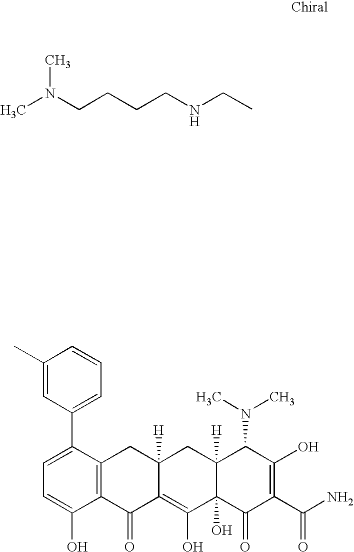 | CV | >134.59 | >134.59 | >134.59 | >134.59 |
| 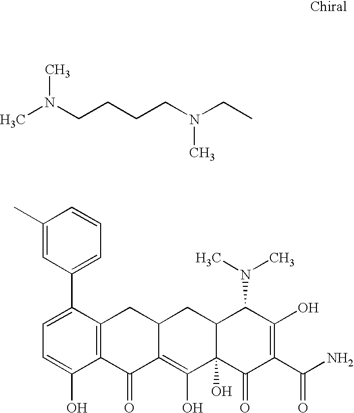 | CW | >132.05 | >132.05 | >132.05 | >132.05 |
| 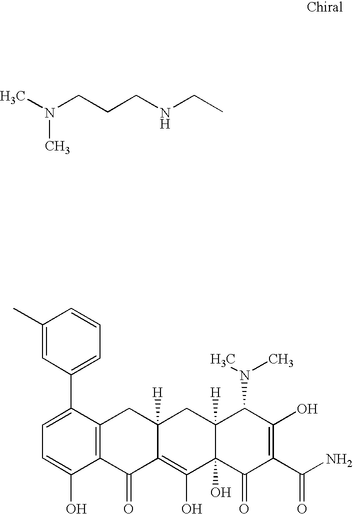 | CX | >137.23 | >137.23 | >137.23 | >137.23 |
| 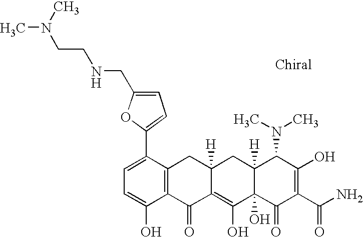 | CY | >142.02 | >142.02 | >142.02 | >142.02 |
| 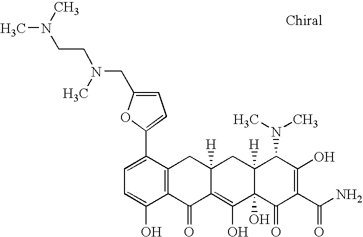 | CZ | >13.77 | >13.77 | >137.77 | >137.77 |
| 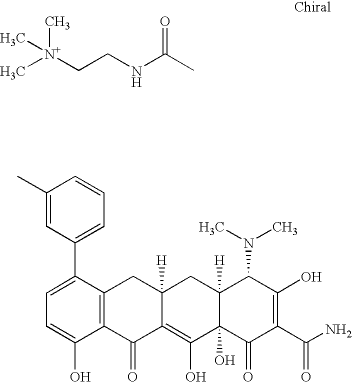 | DA | >1.23 | >1.23 | >123.61 | >123.61 |
| 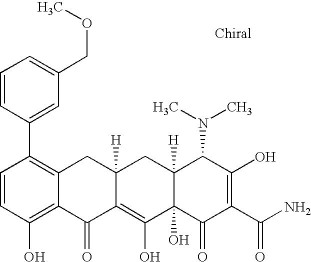 | DB | 10.4 | 13.17 | — | — |
| 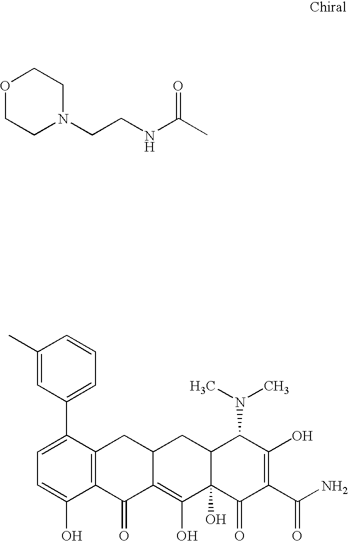 | DC | >13.61 | >13.61 | >136.18 | >136.18 |
| 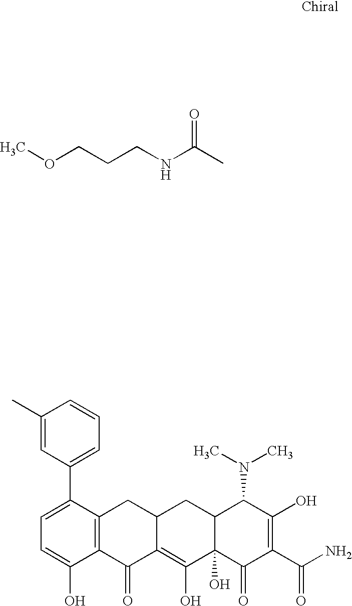 | DD | >15.26 | >15.26 | >152.62 | >152.62 |
| 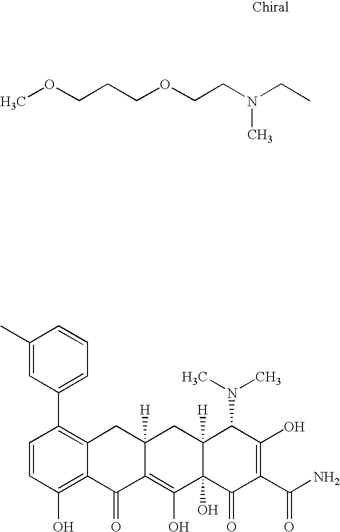 | DE | >20 | >20 | >200 | >200 |
| 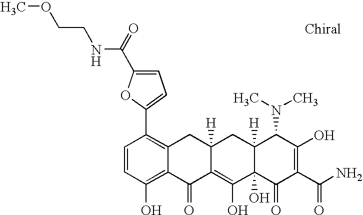 | DF | >20 | >20 | >200 | >200 |
| 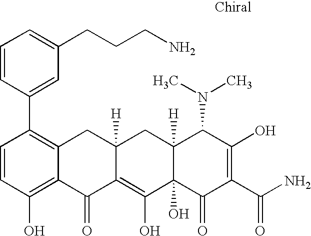 | DG | >20 | >20 | >200 | 64.80181492 |
| 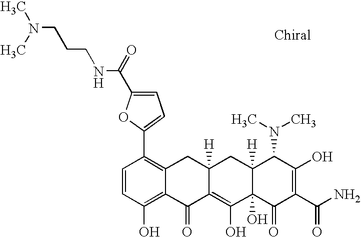 | DH | >200 | >200 | >200 | >200 |
| 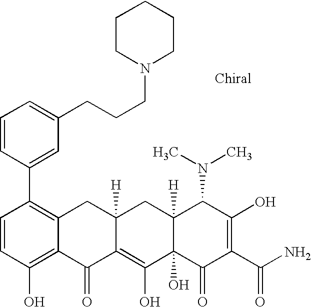 | DI | 59.44 | 54.15 | >200 | 45.65075853 |
| 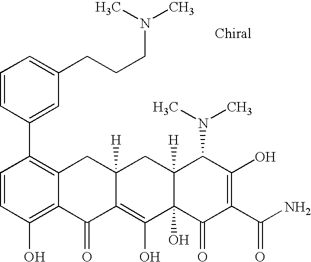 | DJ | 141.46 | 119.32 | >200 | >200 |
| 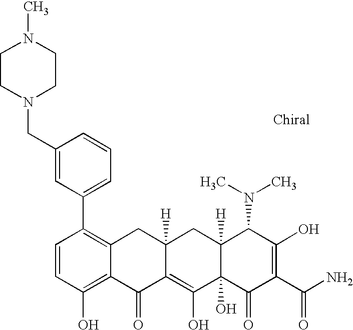 | DK | >200 | 92.34 | >200 | >200 |

### Example 4

### Synthesis of Selected Substituted Tetracycline Compounds

### 7-[2-(4-methyl-piperidin-1-yl)acetyl]-Sancycline (Compound BH)

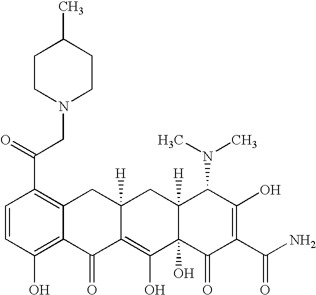

An amount of 7-acetyl sancycline (1 g, 2.19 mmol) was combined with acetic acid (4 mL), water (1 mL) and HBr (33 wt % solution in HOAc) (2 mL, 0.01 mmol) in a 40 mL glass vial. An argon line was attached to the septum and the reaction mixture was stirred until contents dissolved (5 minutes). Bromine (0.15 mL, 1.21 μmol) was added dropwise to reaction solution and an exotherm was detected. The reaction was monitored by HPLC and LC-MS and starting material was consumed within 15 minutes. Mono and bis-substituted bromine products were both detected. The reaction solution was precipitated in 400 mL diethyl ether and a bright yellow solid formed. The ether was decanted and 400 mL fresh ether added, and decanted once again. An amount of acetonitrile (300 mL) was added to the yellow precipitate and the mixture was filtered through filter paper. The filtrate was dried in vacuo to yield a dark yellow solid (1 g). The crude bromo-acetyl sancycline was dissolved in DMF (20 mL) in a 100 mL round bottom flask. The argon line was attached to reaction and TEA (1 mL, 7.19 mmol) was added, followed by 4-methylpiperidine (1 mL, 8.1 mmol). The reaction was monitored by HPLC and LC-MS. Methanol (50 mL) was added to quench the reaction, and the solvent was dried in vacuo. The crude material was purified in 3 batches on a 2″ C-18 Luna column using a 10-30% organic gradient (CH₃CN with 0.1% TFA and water with 0.1% TFA) over 35 minutes. The purified compound was dried in vacuo and redissolved in methanol (20 mL) saturated with HCl to exchange the salt. The compound was dried overnight over P₂O₅ to yield BH (10 mg, 11%) as a yellow powder. MS: (m/z) 553. ¹H NMR (CD₃OD) δ 7.99 (1H, m), 6.93 (1H, m), 4.89 (1H, m), 4.61 (1H, m), 4.07 (1H, s), 3.68 (1H, m), 3.56 (1H, m), 3.30 (1H, m), 3.11 (2H, m), 3.01 (7H, m), 2.47 (1H, m), 2.15 (1H, m), 1.89 (2H, m), 1.55 (4H, m), 0.96 (3H, d, J=9 Hz). Compounds AG, AJ, AM, BB, BO, BP, BR, BS, BT, BU, BV, BW, BX, BY, BZ, CA, CB, CC, CE, CF and CH were prepared in a similar manner.

### 3-[3-((6aS,10S,10aS,11aR)-8-Carbamoyl-10-dimethylamino-4,6,6a,9-tetrahydroxy-5,7-dioxo-5,6a,7,10,10a,11,11a,12-octahydro-naphthacen-1-yl)-benzoylamino]-propionic acid ethyl ester (Compound I)

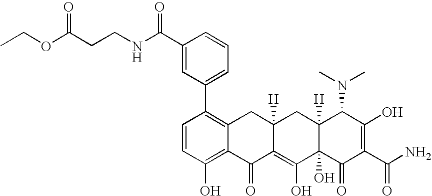

An amount 1.00 g of 7-iodosancycline trifluoroacetic acid salt, 177 mg of palladium (0) tetrakistriphenylphosphine, 35 mg of palladium (II) acetate and 457 mg of 3-(3-ethoxy-3-pxopropylcarbamoyl)phenylboronic acid, 98% were loaded in a dry 20 mL microwave reaction vessel equipped with a magnetic stir bar. Dry dimethylacetamide (DMA, 10 mL) was added and argon was bubbled through the solution for 5 minutes. In a separate vial, sodium acetate (487 mg) was dissolved in distilled water (5 mL) and argon was bubbled through the solution for 5 minutes. The sodium acetate solution was added to the microwave reaction vessel which was sealed with a crimper. The reaction mixture was then subjected to microwave irradiation for 10 minutes at 110° C., and the reaction was monitored by LC/MS. The reaction mixture was filtered through a pad of celite and washed with methanol. After evaporation of organic solvents, the aqueous solution was purified on a fluorinated DVB (divinylbenzene) column with gradients of a 50/50 methanol/acetonitrile, 0.1% TFA solution into a 0.1% TFA water solution. The fractions were collected and evaporated to a minimum volume. The residue was then purified by preparative HPLC chromatography (C18, linear gradient 27-32% acetonitrile in water with 0.2% formic acid). The fractions were evaporated and the resulting residue was purified again by preparative HPLC chromatography (C18, linear gradient 20-35% acetonitrile in 20 mM aqueous triethanolamine, pH 7.4) in order to separate the 4-epimers. The fractions were collected and the organic solvent was evaporated. The resulting aqueous solution was loaded on a DVB column, washed with distilled water, and then with a 0.1% hydrochloric acid solution. After eluting with a 50/50 mixture of methanol and acetonitrile, the solution was evaporated and the residue dried under high vacuum and P₂O₅ overnight to yield a yellow solid as an HCl salt. ESIMS: m/z 634 (MH+). 1H-NMR (300 MHz, tetramethylsilane (TMS) as internal standard at 0 ppm): (ppm) 7.78 (dm, 1H), 7.70 (m, 1H), 7.51 (t, 1H), 7.45 (d, 2H), 6.92 (d, 1H), 4.13 (q, 2H), 4.00 (s, 1H), 3.63 (t, 2H), 2.97-2.80 (m, 8H), 2.77 (dd, 1H), 2.64 (t, 2H), 2.52 (t, 1H), 2.08-1.95 (m, 1H), 1.53 (q, 1H), 1.23 (t, 3H). Compounds A, B, C, D, F, G, H, J, L, P, W, Y, AA, AB, AC, AD, AE, AF, AO, AQ, AR, AS, AT, AU, AW, AX, AY, AZ AP, BC, BE, BF, BG, BI, BJ, BK, BL, BM, BN, B1, CO, CK and CM were prepared in a similar manner.

### (4S,4aS,5aR,12aS)-4-Dimethylamino-7-[(4-dimethylamino-butylamino)-methyl]-3,10,12,12a-tetrahydroxy-1,11-dioxo-1,4,4a,5,5a,6,11,12a-octahydro-naphthacene-2-carboxylic acid amide. (Compound CN)

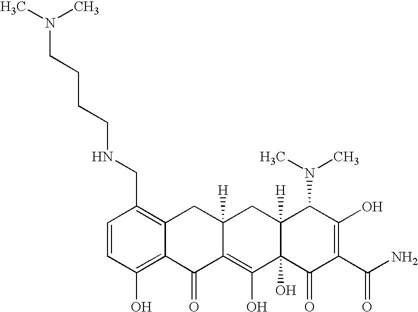

The TFA salt of 7-formyl-sancycline (50 mg, 0.09 mmol) was dissolved in dry tetrahydrofuran (THF, 2 mL) at room temperature in a flask equipped with a magnetic stirring bar. Enough di-isopropylethylamine (DIEA) was added to adjust the pH to about 7. N,N-Dimethyl-4-amino-butylamine (22 mg, 0.18 mmol, 2.0 eq) was added and the reaction mixture was stirred at room temperature for 15 minutes. Sodium triacetoxyborohydride (59 mg, 0.27 mmol, 3.0 eq) was added at room temperature and the reaction is monitored by LC/MS. After 2 hours, the reaction was completed and after filtration of the mixture, the residue was purified by preparative HPLC (C18, linear gradient acetonitrile in water with 0.2% formic acid). The fractions were combined, evaporated and the resulting residue was purified again by preparative HPLC chromatography (C18, linear gradient acetonitrile in 20 mM aqueous triethanolamine, pH 7.4) in order to separate the 4-epimers. The fractions were collected and the organic solvent evaporated. The resulting aqueous solution was loaded on a DVB column, washed with DI water and then with a 0.1% hydrochloric acid solution. After eluting with a 50/50 mixture of methanol and acetonitrile, the solution was evaporated and the residue dried under high vacuum and P₂O₅ overnight to yield a yellow solid as an HCl salt. ¹H-NMR (chemical shifts in ppm with TMS as internal reference at 0 ppm, in deuterated methanol): δ 7.64 (1H, doublet, aromatic), δ 6.92 (1H, doublet, aromatic), δ 4.25 (2H, singlet), δ 4.12 (1H, singlet), δ 3.30-2.80 (19H, multiplet), δ 2.48 (1H, multiplet), δ 2.35 (1H, multiplet), δ 1.85 (4H, multiplet), δ 1.62 (1H, multiplet). Mass Spectroscopy (Electron Spray): M+1=543. Compound AK was prepared in a similar manner.

### (4S,4aS,5aR,12aS)-7-[3-(2-Diethylamino-ethylcarbamoyl)-phenyl]-4-dimethylamino-3,10,12,12a-tetrahydroxy-1,1′-dioxo-1,4,4a,5,5a,6,11,12a-octahydro-naphthacene-2-carboxylic acid amide (Compound E)

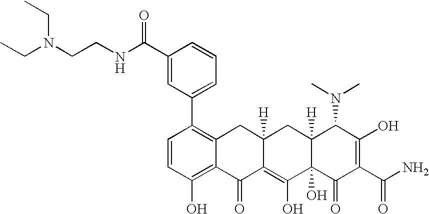

An amount of 2.5 g of 7-iodosancycline trifluoroacetic acid salt, 221 mg of palladium (0) tetrakistriphenylphosphine, 43 mg of palladium (II) acetate and 777 mg of 3-carboxy-phenylboronic acid were loaded in a dry 20 mL microwave reaction vessel equipped with a magnetic stir bar. Dry DMA (13 mL) was added and argon was bubbled through the solution for 5 minutes. In a separate vial, sodium acetate (105.99 g/mol, 1.215 g, 11.46 mmol, 3.0 eq.) was dissolved in distilled water (7 mL) and argon was bubbled through the solution for 5 minutes. The sodium acetate solution was added to the microwave reaction vessel, which was sealed with a crimper. The reaction mixture was then subjected to microwave irradiation for 10 minutes at 110° C., and the reaction was monitored by LC/MS. The reaction mixture was filtered through a pad of celite and washed with methanol. After evaporation of organic solvents, the aqueous solution was purified on a fluorinated DVB (divinylbenzene) column with gradients of a 50/50 methanol/acetonitrile, 0.1% TFA solution into a 0.1% TFA water solution. The fractions were collected and evaporated to dryness to yield an orange solid, which was used in the next step without further purification.

An amount of 340 mg of 7-(3-carboxy-phenyl)-sancycline TFA salt and 212 mg of O-benzotriazol-1-yl-N,N,N′N′-tetramethyluronium hexafluoro-phosphate were loaded in a dry 10 mL vial equipped with a magnetic stir bar. Dry DMA (2.5 mL) was added, followed by diisopropylethylamine (180 μL). After 5 minutes of stirring at room temperature, N,N-diethyl-ethylenediamine, 98% (150 μL) was added, the reaction mixture was stirred at room temperature for 15 minutes and the reaction was monitored by LC/MS. The mixture was filtered through celite, evaporated in a rotary evaporator, and the residue was purified by preparative HPLC chromatography (C18, linear gradient 25-35% acetonitrile in water with 0.2% formic acid). The fractions were combined, evaporated, and the resulting residue was purified again by preparative HPLC chromatography (C18, linear gradient 20-35% acetonitrile in 20 mM aqueous triethanolamine, pH 7.4) in order to separate the 4-epimers. The fractions were collected and the organic solvent evaporated. The resulting aqueous solution was loaded on a DVB column, washed with DI water, and then washed with a 0.1% hydrochloric acid solution. After eluting with a 50/50 mixture of methanol and acetonitrile, the solution was evaporated and the residue dried under high vacuum and P₂O₅ overnight to yield a yellow solid as an HCl salt. ESIMS: m/z 633 (MH+). 1H-NMR (300 MHz, tetramethylsilane (TMS) as internal standard at 0 ppm): (ppm) 7.87 (dm, 1H), 7.79 (m, 1H), 7.60-7.47 (m, 2H), 7.44 (d, 1H), 6.93 (d, 1H), 4.02 (s, 1H), 3.76 (t, 2H), 3.45-3.30 (m, 6H), 3.02-2.85 (m, 8H), 2.78 (dd, 1H), 2.54 (t, 1H), 2.10-1.95 (m, 1H), 1.53 (q, 1H), 1.35 (t, 6H). Compounds M, N, O, R, S, T, U, CL, CP, CQ and CR were prepared in a similar manner.

### (4S,4aS,5aR,12aS)-4-Dimethylamino-7-(3-{[(3-dimethylamino-propyl)-methyl-amino]-methyl}-phenyl-3,10,12,12a-tetrahydroxy-1,11-dioxo-1,4,4a,5,5a,6,11,12a-octahydro-naphthacene-2-carboxylic acid amide (Compound CU)

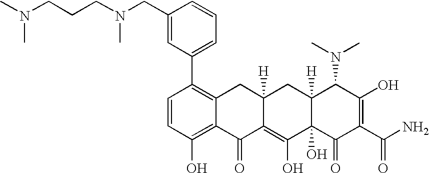

The TFA salt of 7-(3-formyl)-phenyl-sancycline (200 mg, 0.32 mmol) was dissolved in dry dimethylacetamide (DMA, 2 mL) at room temperature in a flask equipped with a magnetic stirring bar. Enough di-isopropylethylamine (DIEA) was added to adjust the pH to about 7. N,N,N′-Trimethyl-3-amino-propylamine (46 mg, 0.40 mmol) was added and the reaction mixture is stirred at room temperature for 15 minutes. Sodium triacetoxyborohydride (83 mg, 0.39 mmol, 1.2 eq) was added at room temperature and the reaction was monitored by LC/MS. After 2 hours, the reaction was complete and after filtration of the mixture, the residue was purified by preparative HPLC (C18, linear gradient 15-35% acetonitrile in water with 0.2% formic acid). The fractions were combined, evaporated, and the resulting residue was purified again by preparative HPLC chromatography (C18, linear gradient 15-35% acetonitrile in 20 mM aqueous triethanolamine, pH 7.4) in order to separate the 4-epimers. The fractions were collected and the organic solvent evaporated. The resulting aqueous solution was loaded on a DVB column, washed with DI water, and then with a 0.1% hydrochloric acid solution. After eluting with a 50/50 mixture of methanol and acetonitrile, the solution was evaporated and the residue dried under high vacuum and P₂O₅ overnight to yield a yellow solid as an HCl salt. ¹H-NMR (chemical shifts in ppm with TMS as internal reference at 0 ppm, in deuterated methanol): δ 7.60-7.30 (5H, multiplet, aromatic), δ 7.12 (1H, doublet, aromatic), δ 4.28 (2H, singlet), δ 4.09 (1H, singlet), δ 3.17 (4H, multiplet), δ 3.05-3.75 (18H, multiplet), δ 2.54 (1H, multiplet), δ 2.09 (1H, multiplet), δ 1.83 (2H, multiplet), δ 1.53 (1H, multiplet). Mass Spectroscopy (Electron Spray): M+1=619. Compounds AX, AY, AZ, BF, BI, BK, BQ, CS, CT, CV, CW, CX were prepared in a similar manner.

### (4S,4aS,5aR,12aS)-4,7-Bis-dimethylamino-9-[3-(2-dimethylamino-ethylcarbamoyl)-phenyl]-3,10,12,12a-tetrahydroxy-1,11-dioxo-1,4,4a,5,5a,6,11,12a-octahydro-naphthacene-2-carboxylic acid amide (Compound V)

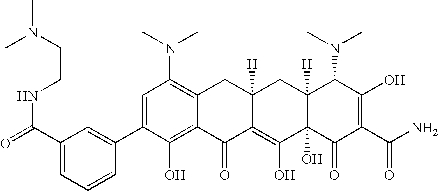

An amount of 500 mg of 9-iodo-minocycline free base, 100 mg of palladium (0) tetrakis triphenylphosphine, 20 mg of palladium (II) acetate and 234 mg of [3-(3-N,N-dimethylaminoetylaminocarbonyl)-phenyl]-boronic acid were loaded in a dry 20 mL microwave reaction vessel equipped with a magnetic stir bar. Dry DMA (4 mL) was added and argon was bubbled through the solution for 5 minutes. In a separate vial, sodium acetate (274 mg) was dissolved in DI water (2 mL) and argon was bubbled through the solution for 5 minutes. The sodium acetate solution was added to the microwave reaction vessel, which was sealed with a crimper. The reaction mixture was then subjected to microwave irradiation for 10 minutes at 110° C., and the reaction was monitored by LC/MS. The reaction mixture was filtered through a pad of celite and washed with methanol. After evaporation of organic solvents, the aqueous solution was purified on a fluorinated DVB (DiVinylBenzene) column with gradients of a 50/50 methanol/acetonitrile, 0.1% TFA solution into a 0.1% TFA water solution. The fractions were collected and evaporated to a minimum volume. The residue was then purified by HPLC chromatography (C18, linear gradient 10-20% acetonitrile in water with 0.2% formic acid). The fractions were combined, evaporated, and the resulting residue was purified again by preparative HPLC chromatography (C18, linear gradient 10-20% acetonitrile in 20 mM aqueous triethanolamine, pH 7.4) in order to separate the 4-epimers. The fractions were collected and the organic solvent evaporated. The resulting aqueous solution was loaded on a DVB column, washed with distilled water, and then with a 0.1% hydrochloric acid solution. After eluting with a 50/50 mixture of methanol and acetonitrile, the solution was evaporated and the residue dried under high vacuum and P₂O₅ overnight to yield a yellow solid as an HCl salt. ESIMS: m/z 648 (MH+). 1H-NMR (300 MHz, tetramethylsilane (TMS) as internal standard at 0 ppm): (ppm) 8.26 (t, 1H), 8.16 (s, 1H), 7.94 (m, 2H), 7.59 (t, 1H), 4.19 (s, 1H), 3.82 (t, 2H), 3.50-3.30 (m, 9H), 3.30-3.10 (m, 2H), 3.10-2.90 (m, 9H), 2.62 (t, 1H), 2.42-2.30 (m, 1H), 1.71 (q, 1H). Compound X, BA and CD were prepared in a similar manner.

### (4aS,5aR,12aS)-7-[3-(2-Dimethylamino-ethylcarbamoyl)-phenyl]-3,10,12,12a-tetrahydroxy-1,11-dioxo-1,4,4a,5,5a,6,11,12a-octahydro-naphthacene-2-carboxylic acid amide (Compound Z)

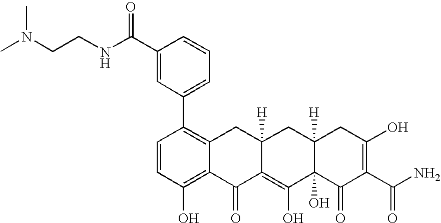

An amount of 1.00 g of 7-iodo-4-dedimethylamino-sancycline free base, 233 mg of palladium (0) tetrakis triphenylphosphine, 45 mg of palladium (II) acetate and 544 mg of [3-(3-N,N-dimethylaminoethylaminocarbonyl)-phenyl]-boronic acid were loaded in a dry 20 mL microwave reaction vessel equipped with a magnetic stir bar. Dry DMA (8 mL) was added and argon was bubbled through the solution for 5 minutes. In a separate vial, sodium acetate (640 mg) was dissolved in distilled water (4 mL) and argon was bubbled through the solution for 5 minutes. The sodium acetate solution was added to the microwave reaction vessel, which was sealed with a crimper. The reaction mixture was then subjected to microwave irradiation for 10 minutes at 110° C. and the reaction was monitored by LC/MS. The reaction mixture was filtered through a pad of celite and washed with methanol. After evaporation of organic solvents, the aqueous solution was purified on a fluorinated DVB (DiVinylBenzene) column with gradients of a 50/50 methanol/acetonitrile, 0.1% TFA solution into a 0.1% TFA water solution. The fractions were collected and evaporated to a minimum volume. The residue was then purified by preparative HPLC chromatography (C18, linear gradient 20-35% acetonitrile in water with 0.2% formic acid). The fractions were combined, evaporated, and the resulting residue was purified again by preparative HPLC chromatography (C18, linear gradient 15-35% acetonitrile in 20 mM aqueous triethanolamine, pH 7.4) in order to separate the 4-epimers. The fractions were collected and the organic solvent evaporated. The resulting aqueous solution was loaded on a DVB column, washed with distilled water, and then with a 0.1% hydrochloric acid solution. After eluting with a 50/50 mixture of methanol and acetonitrile, the solution was evaporated and the residue dried under high vacuum and P₂O₅ overnight to yield a yellow solid as an HCl salt. ESIMS: m/z 562 (MH+). 1H-NMR (300 MHz, tetramethylsilane (TMS) as internal standard at 0 ppm): (ppm) 7.87 (dm, 1H), 7.78 (s, 1H), 7.60-7.45 (m, 2H), 7.41 (d, 1H), 6.90 (d, 1H), 3.76 (m, 2H), 3.38 (t, 2H), 3.21 (dd, 1H), 2.98 (s, 6H), 2.85-2.62 (m, 2H), 2.57-2.22 (m, 3H), 1.90-1.80 (m, 1H), 1.48 (q, 1H).

### (4S,4aS,5aR,12aS)-4-Dimethylamino-7-[3-(2-dimethylamino-acetylamino)-phenyl]-3,10,12,12a-tetrahydroxy-1,11-dioxo-1,4,4a,5,5a,6,11,12a-octahydro-naphthacene-2-carboxylic acid amide (Compound K)

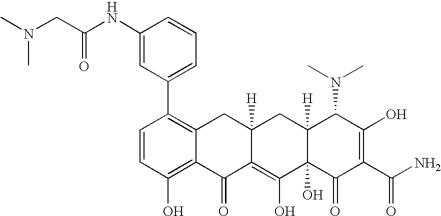

An amount of 2.50 g of 7-iodosancycline trifluoroacetic acid salt, 221 mg palladium (0) tetrakis triphenylphosphine, 42 mg of palladium (II) acetate, and 812 mg of 3-amino-phenylboronic acid were loaded in a dry 20 mL microwave reaction vessel equipped with a magnetic stir bar. Dry DMA (13 mL) was added and argon was bubbled through the solution for 5 minutes. In a separate vial, sodium acetate (1.22 g) was dissolved in distilled water (7 mL) and argon was bubbled through the solution for 5 minutes. The sodium acetate solution was added to the microwave reaction vessel, which was sealed with a crimper. The reaction mixture was then subjected to microwave irradiation for 20 minutes at 120° C., and the reaction was monitored by LC/MS. The reaction mixture was then filtered through a pad of celite and washed with methanol. After evaporation of organic solvents, the aqueous solution was purified on a fluorinated DVB (DiVinylBenzene) column with gradients of a 50/50 methanol/acetonitrile, 0.1% TFA solution into a 0.1% TFA water solution. The fractions were collected and evaporated to dryness to yield a brown solid which is used in the next step without further purification.

An amount of 250 mg of 7-(3-amino-phenyl)-sancycline TFA salt and 250 μL of diisopropylethylamine were loaded into a dry 5 mL microwave reaction vessel equipped with a magnetic stir bar. After 5 minutes of stirring, dimethylamino acetyl chloride, 85% (667 mg) was added, the reaction vessel was sealed, the reaction mixture was subjected to microwave irradiation for 5 minutes at 100° C. and the reaction was monitored by LC/MS. The mixture was filtered through celite, evaporated in a rotary evaporator, and the residue was purified by preparative HPLC chromatography (C18, linear gradient 10-30% acetonitrile in water with 0.2% formic acid). The fractions were combined, evaporated, and the resulting residue was purified again by preparative HPLC chromatography (C18, linear gradient 15-25% acetonitrile in 20 mM aqueous triethanolamine, pH 7.4) in order to separate the 4-epimers. The fractions were collected and the organic solvent evaporated. The resulting aqueous solution was loaded on a DVB column, washed with distilled water, and then with a 0.1% hydrochloric acid solution. After eluting with a 50/50 mixture of methanol and acetonitrile, the solution was evaporated and the residue dried under high vacuum and P205 overnight to yield a yellow solid as an HCl salt. ESIMS: m/z 591 (MH+). 1H-NMR (300 MHz, tetramethylsilane (TMS) as internal standard at 0 ppm): (ppm) 7.56 (m, 2H), 7.45-7.32 (m, 2H), 7.07 (d, 1H), 6.91 (d, 2H), 4.15 (s, 2H), 4.04 (s, 1H), 3.20-2.70 (m, 15H), 2.48 (t, 1H), 2.04 (m, 1H), 1.51, (m, 1H). Compound Q was prepared in a similar manner.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

All patents, patent applications, and literature references cited herein are hereby expressly incorporated by reference.

What is claimed is:

1. A method of treating a microorganism-associated infection in a subject comprising administering to said subject an effective amount of a tetracycline compound, wherein said tetracycline compound is of formula I: 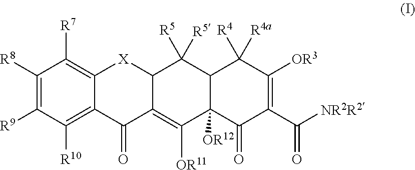
wherein
X is CHC(R¹³Y′Y), CR^{6′}R⁶, C═CR^{6′}R⁶, S, NR⁶, or O;
R², R^{2′}, R^{4′}, and R^{4″} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or a prodrug moiety;
R³, R^{4a}, R¹¹ and R¹² are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or a prodrug moiety;
R⁴ is NR^{4′}R^{4″}, hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or a prodrug moiety;
R⁵ and R^{5′} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or a prodrug moiety;
R⁶ and R^{6′} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;
R⁷ is hydrogen, alkyl, alkenyl, alkynyl, acyl, hydroxyl, alkoxy, halogen, thioether, sulfinyl, sulfonyl, amino, cyano, nitro, carbonyl, oximyl, aryl, heterocyclic or —(CH₂)₀₋₃ (R^{7c})₀₋₁C(═W)WR^{7a};
R⁸ is hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or —(CH₂)₀₋₃(NR^{8c})₀₋₁C(=E′)ER^{8a};
R⁹ is hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or —(CH₂)₀₋₃(NR^{9c})₀₋₁C(=Z′)ZR^{9a};
R¹⁰ is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;
R^{7a}, R^{7b}, R^{7c}, R^{7d}, R^{7e}, R^{7f}, R^{8a}, R^{8b}, R^{8c}, R^{8d}, R^{8e}, R^{8f}, R^{9a}, R^{9b}, R^{9c}, R^{9d}, R^{9e}, and R^{9f} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; R¹³ is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;
E is CR^{8d}R^{8e}, S, NR^{8b} or O;
E′ is O, NR^{8f}, or S;
W is CR^{7d}R^{7e}, S, NR^{7b} or O;
W′ is O, NR^{7f}, or S;
X is CHC(R¹³Y′Y), C═CR¹³Y, CR^{6′}R⁶, S, NR⁶, or O;
Z is CR^{9d}R^{9e}S, NR^{9b} or O;
Z′ is O, S, or NR^{9f};
Y′ and Y are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; or a pharmaceutically acceptable salt, ester or enantiomer thereof;
such that said subject is treated.

2. The method of claim 1, wherein X is CR⁶R^{6′}; R^{2′}, R^{2″}, R³, R^{4a}, R⁵, R^{5′}, R⁶, R^{6′}, R⁸, R⁹, R¹¹ and R¹² are each hydrogen; R⁴ is NR^{4′}R^{4″} and R^{4′} and R^{4″} are each alkyl.

3. The method of claim 2, wherein said alkyl is methyl.

4. The method of claim 1, wherein R⁷ is aryl.

5. The method of claim 1, wherein said aryl is of formula XI: 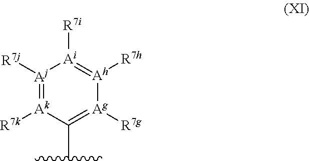
wherein
A^{g}, A^{h}, Aⁱ, A^{j} and A^{k} are each independently N or C; and
when A^{g}, A^{h}, Aⁱ, A^{j} and A^{k} are C; R^{7g}, R^{7h}, R⁷ⁱ, R^{7j} and R^{7k} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or R^{7j} and R⁷ⁱ are linked to form a 5- or 6-membered aryl, heterocyclic or aliphatic ring; or R^{7g}, R^{7h}, R⁷ⁱ, R^{7j} and R^{7k} are absent when A^{g}, A^{h}, Aⁱ, A^{j} and A^{k} are N.

6. The method of claim 5, wherein A^{g}, A^{h}, Aⁱ, A^{j} or A^{k} are each C.

7. The method claim 6, wherein R^{7g}, R^{7h}, R⁷ⁱ and R^{7k} are each hydrogen.

8. The method of claim 7, wherein R^{7j} is carbonyl.

9. The method of claim 1, wherein R⁷ is selected from the group consisting of phenyl, furanyl, piperidinyl, isoquinolinyl, pyridinyl, pyrrolyl, and piperazinyl.

10. The method of claim 1, wherein said tetracycline compound is a compound of formula II, III, IV, V, VI, VII, VIII, IX or X.

11. The method of claim 1, wherein said tetracycline compound is selected from the group consisting of: 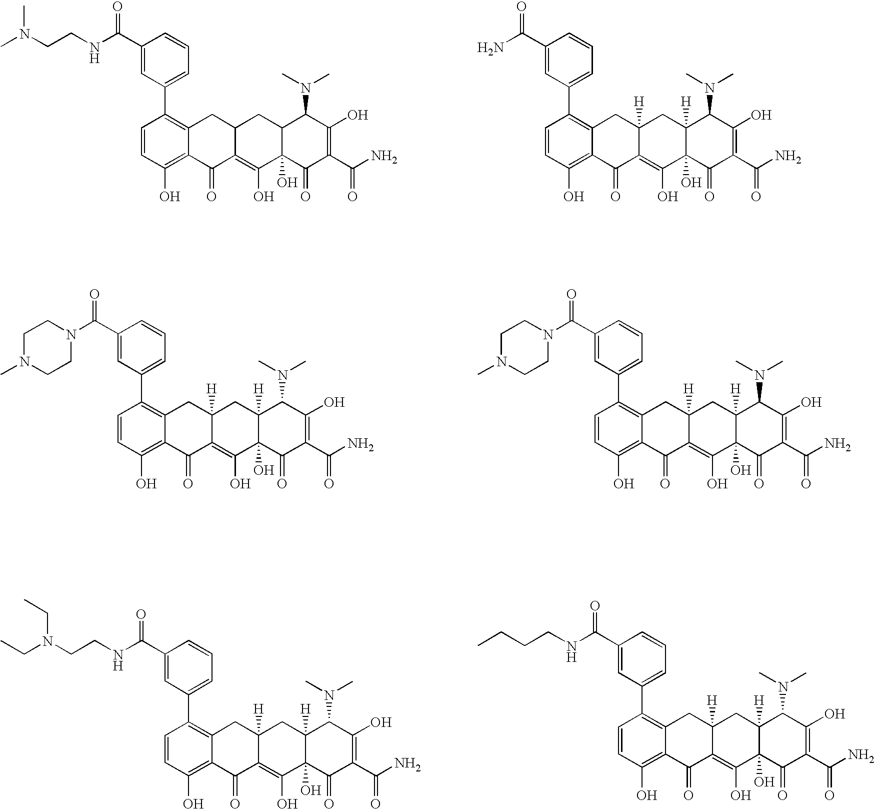 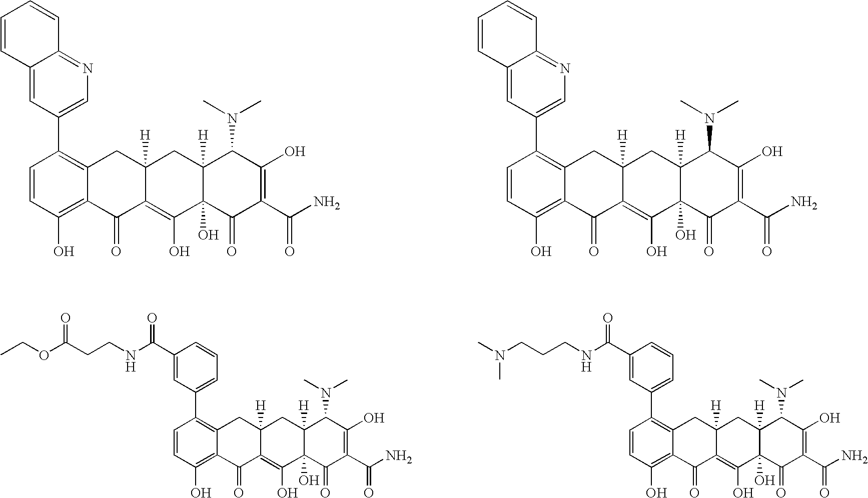 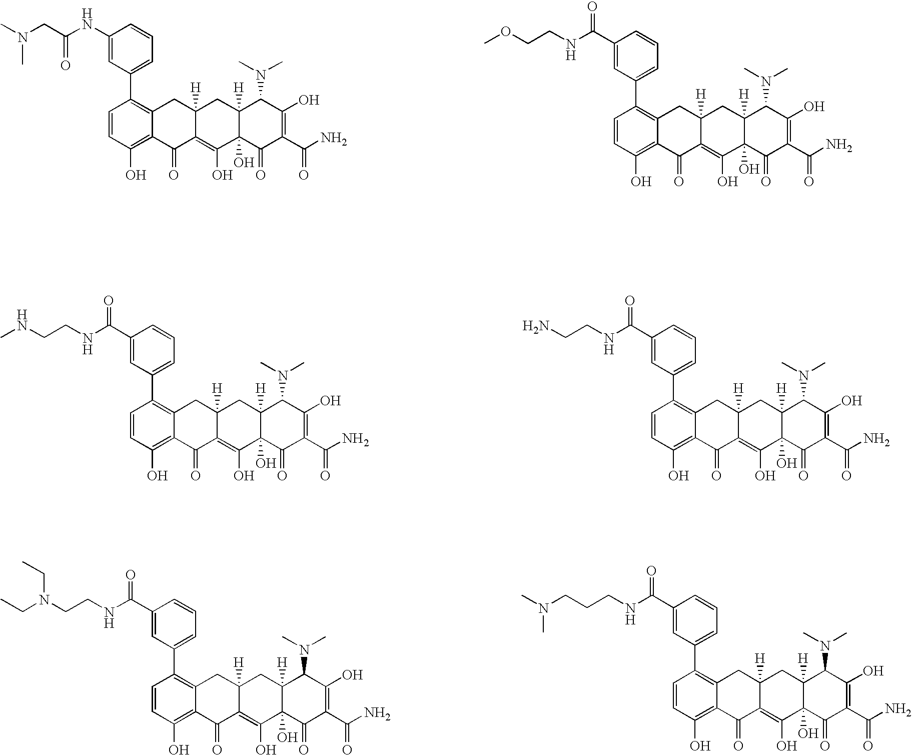 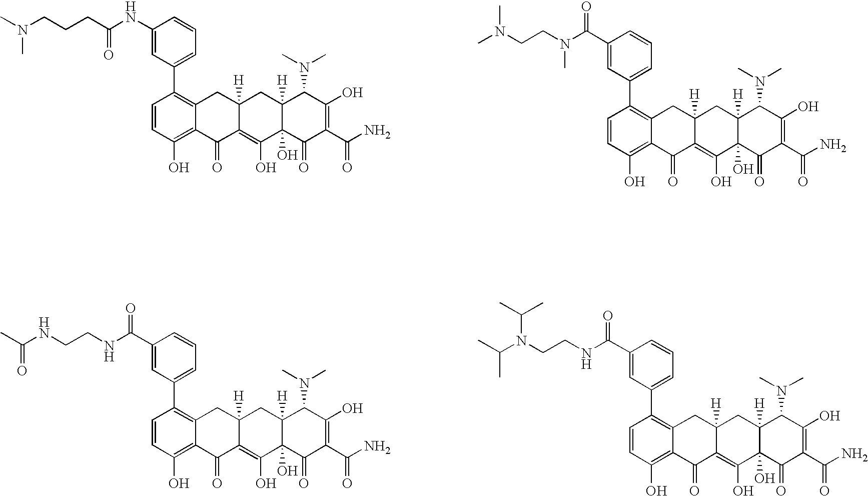 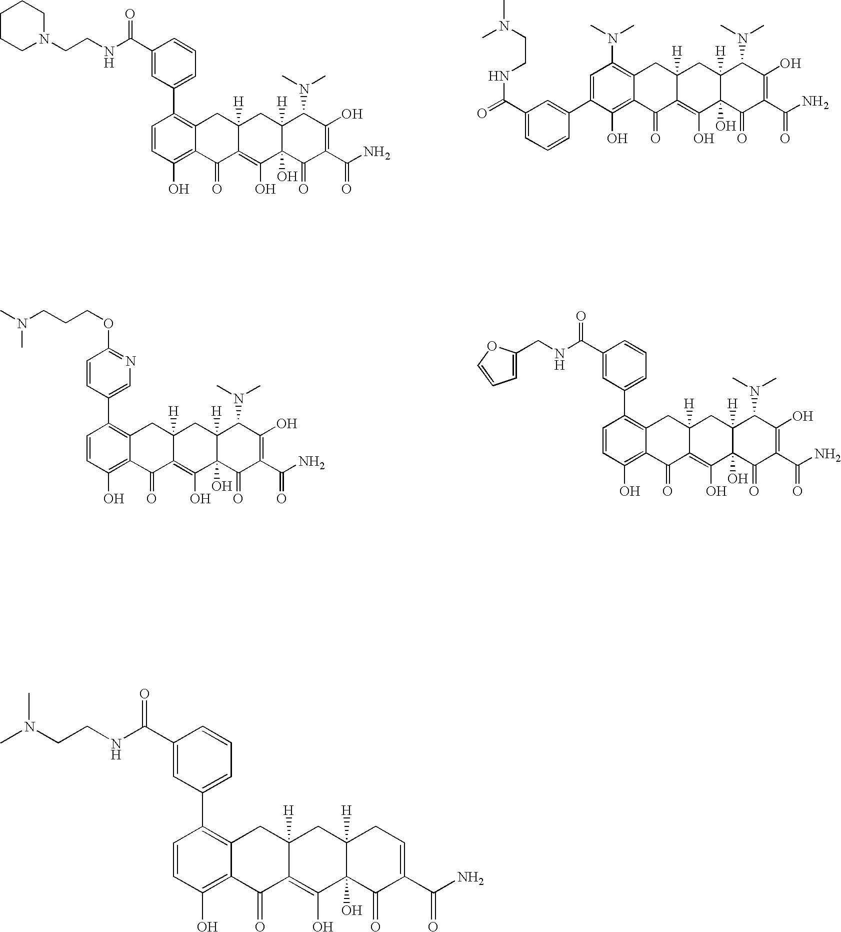 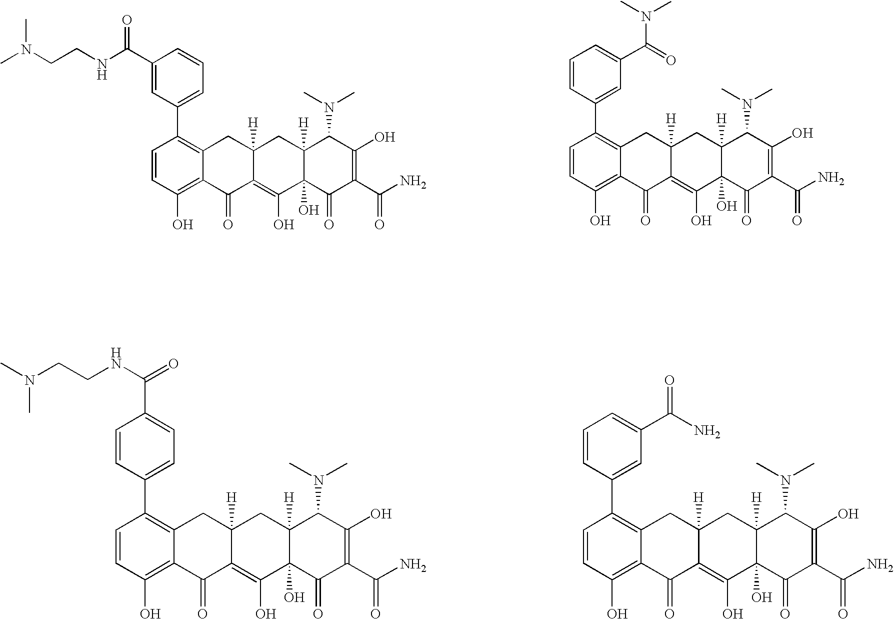 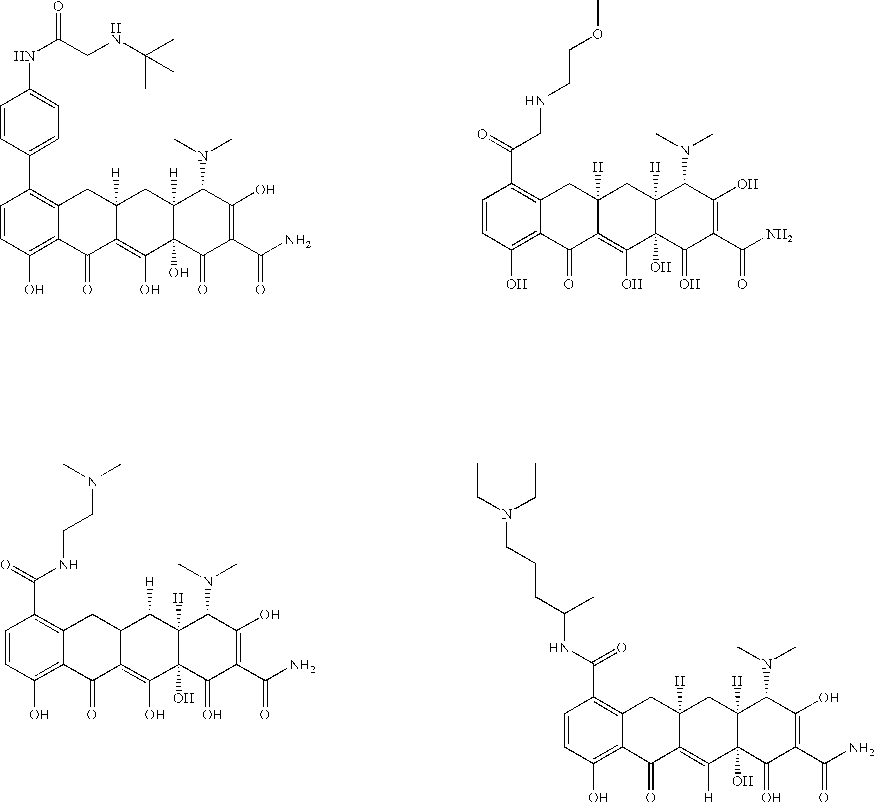 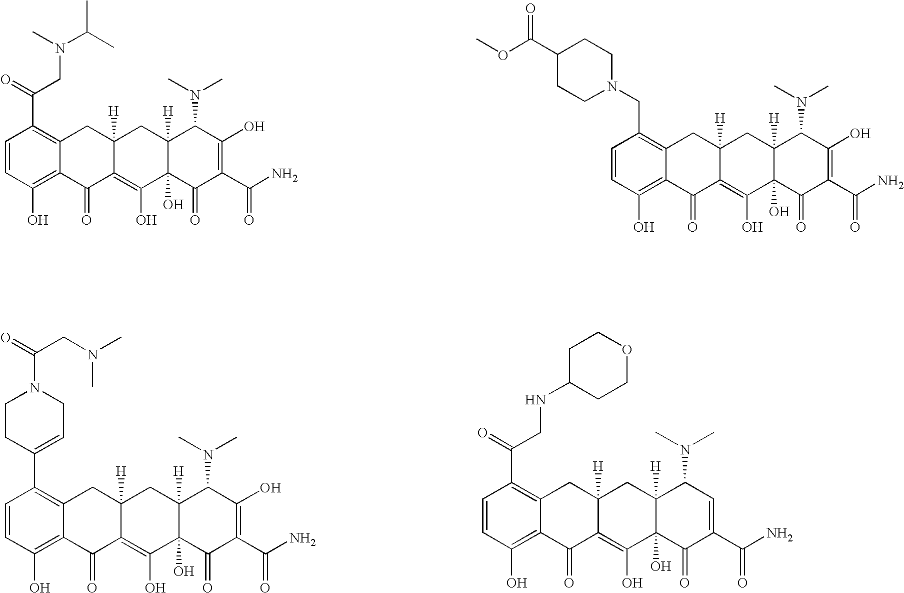 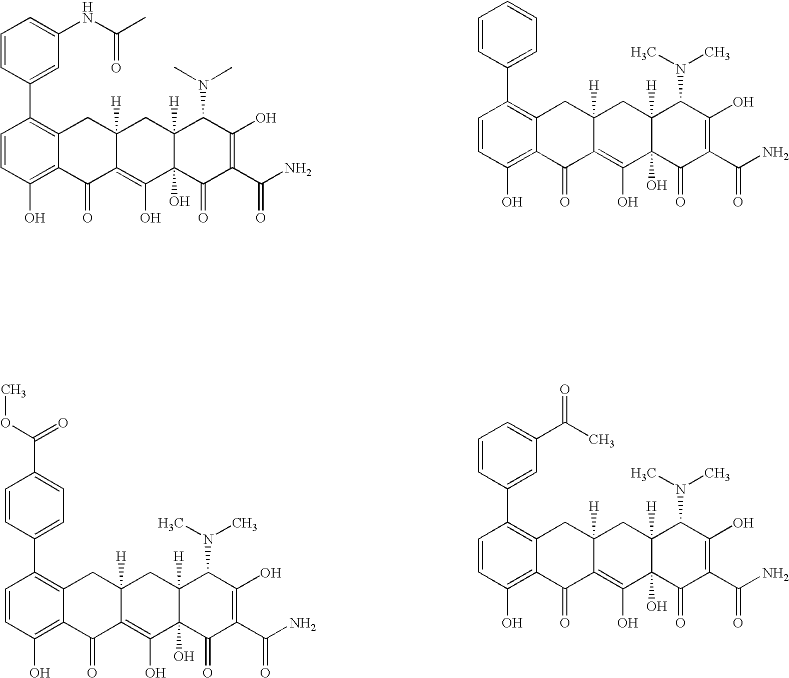 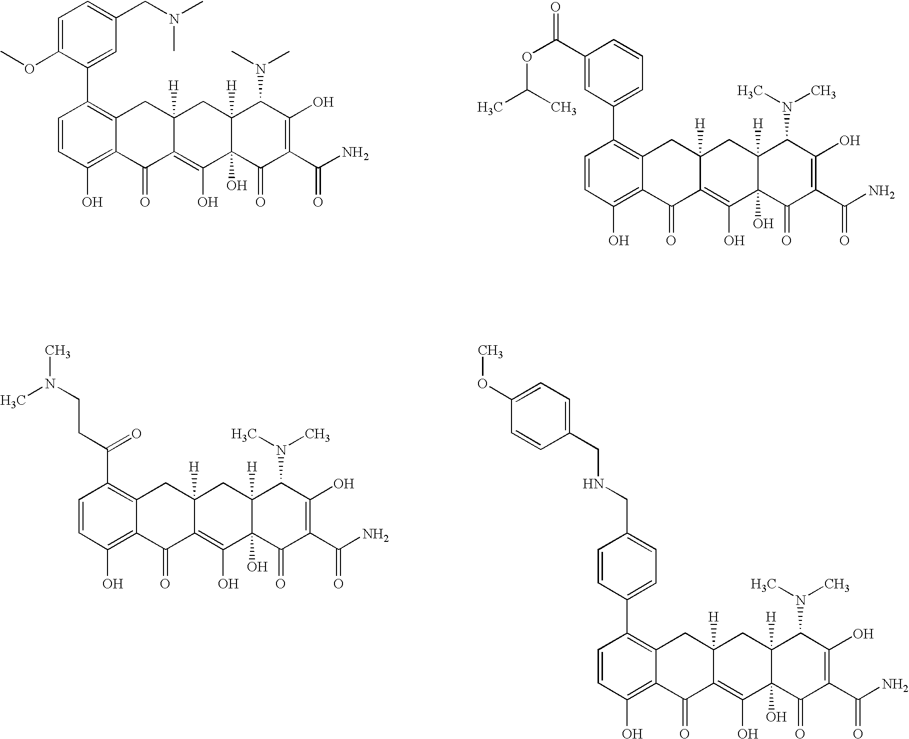 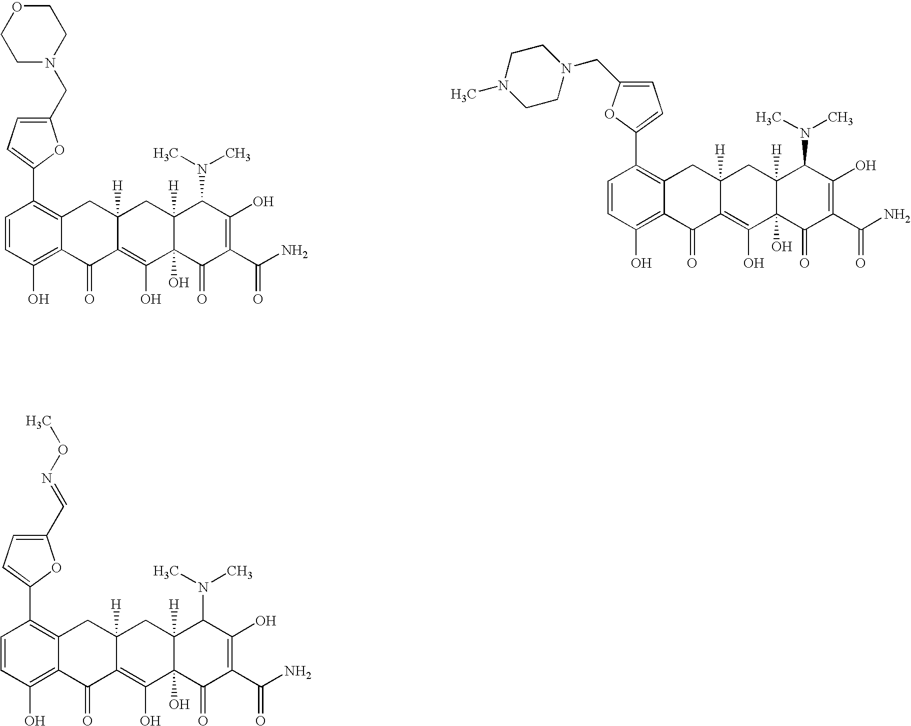 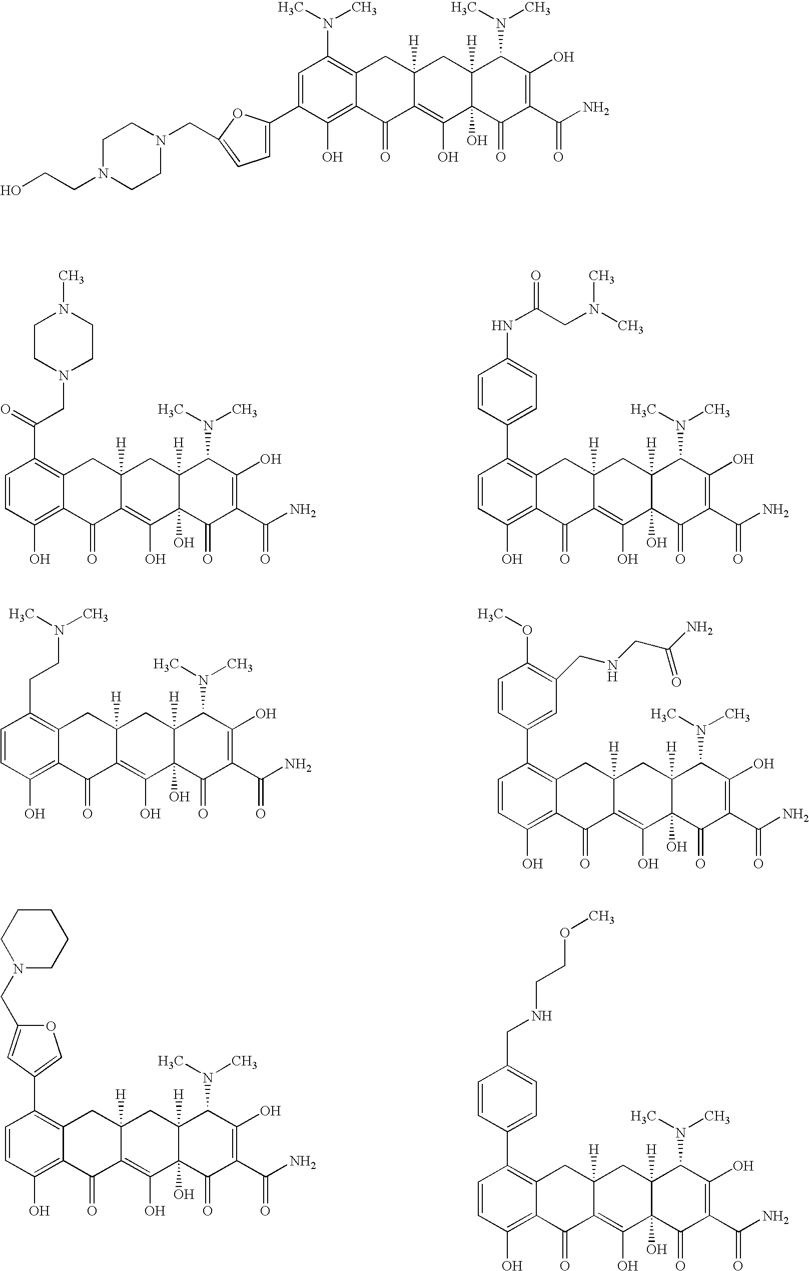 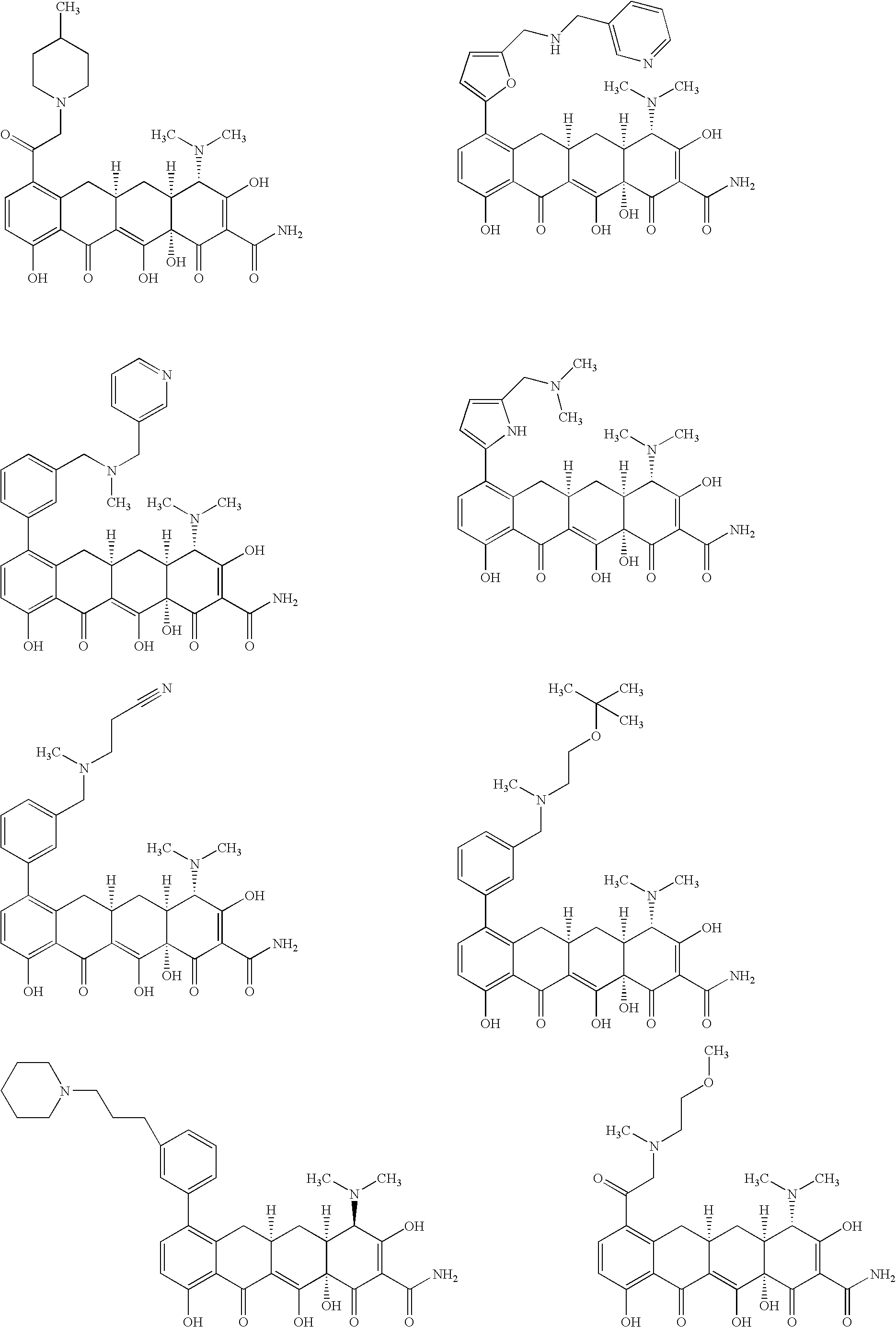 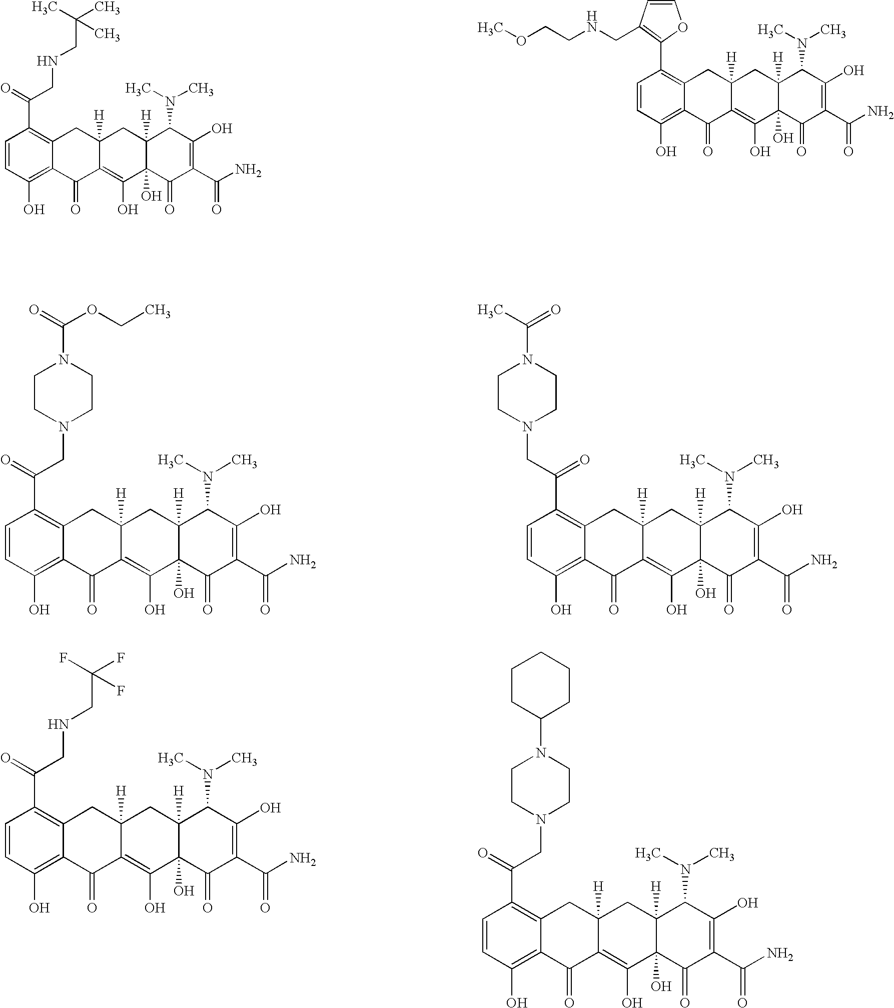 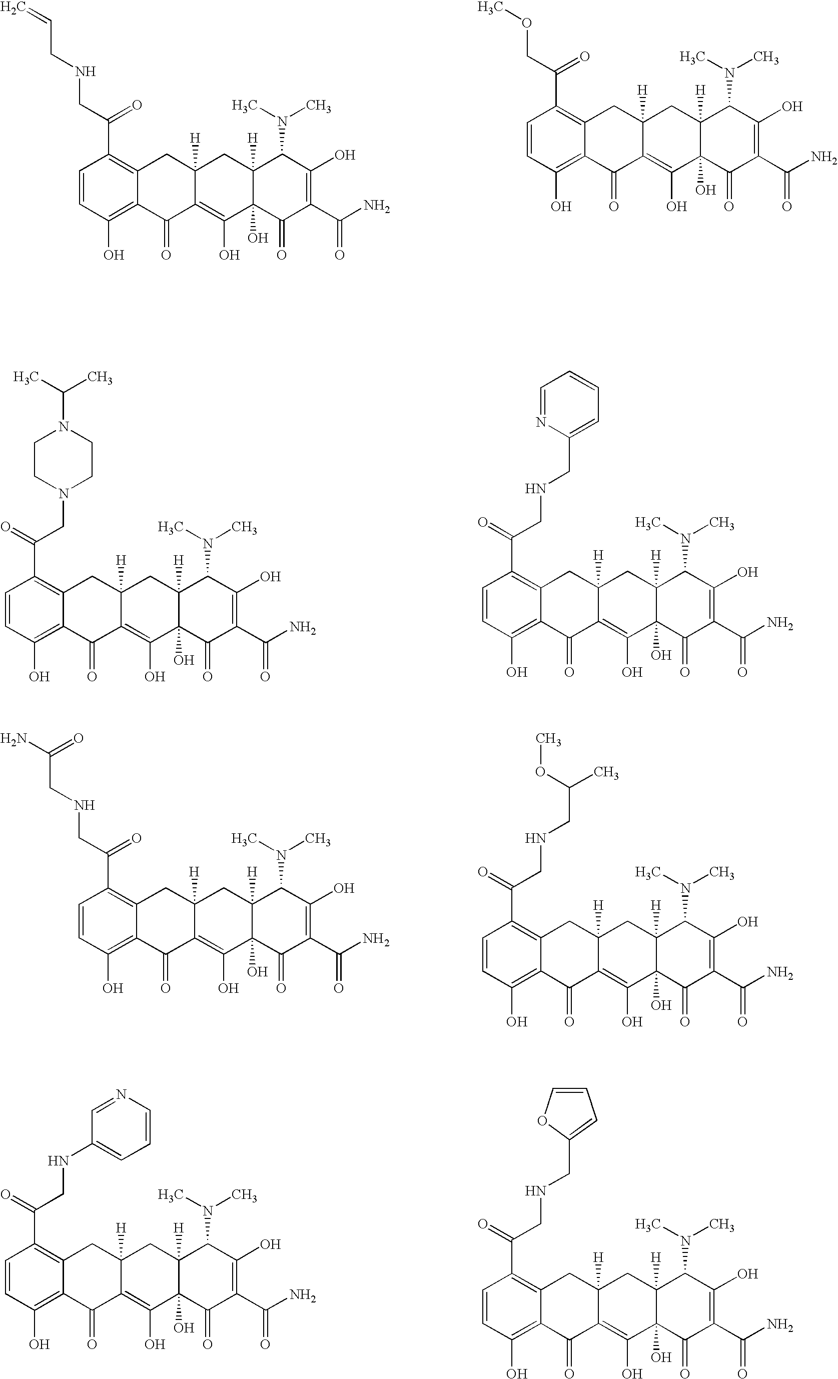 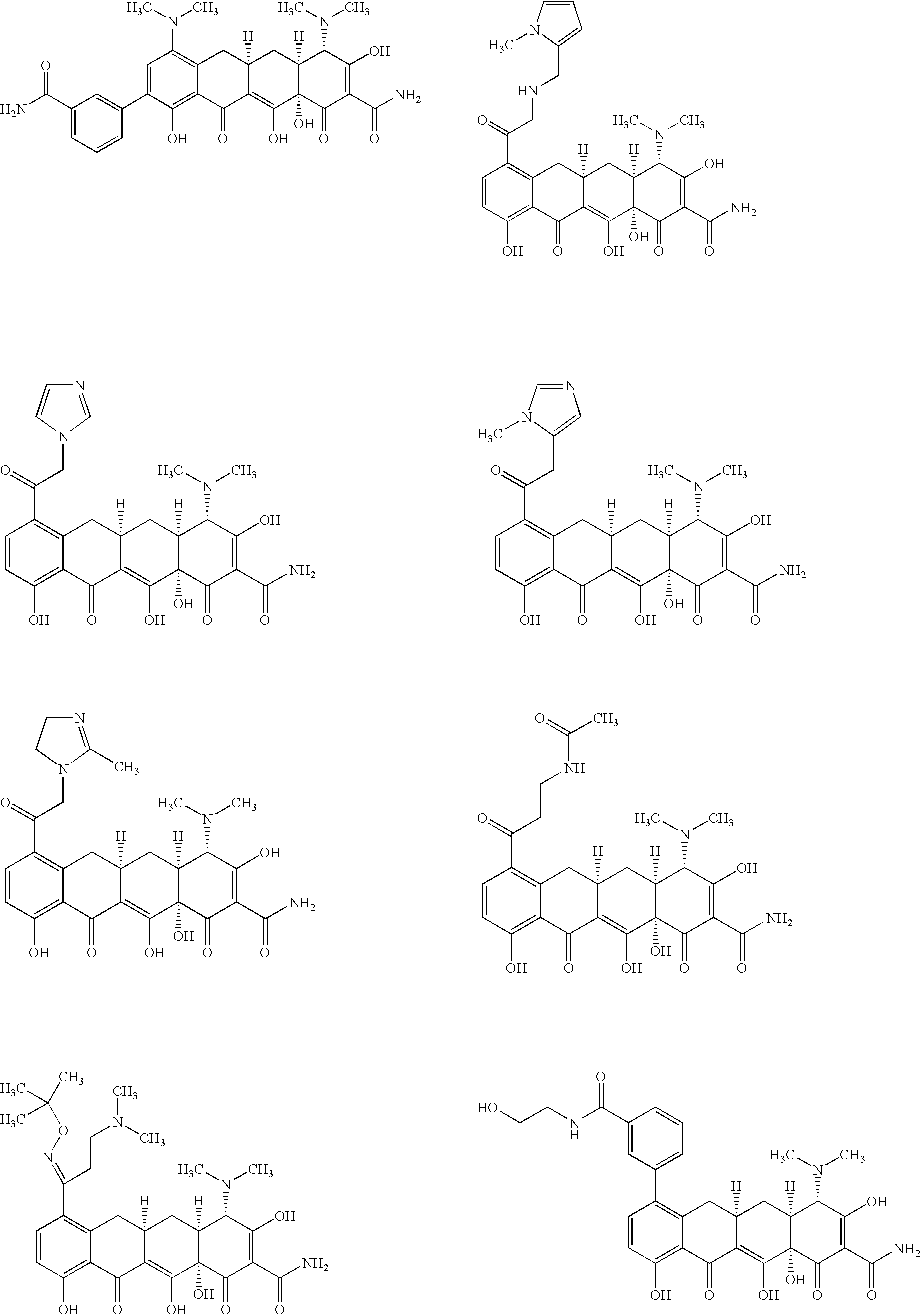 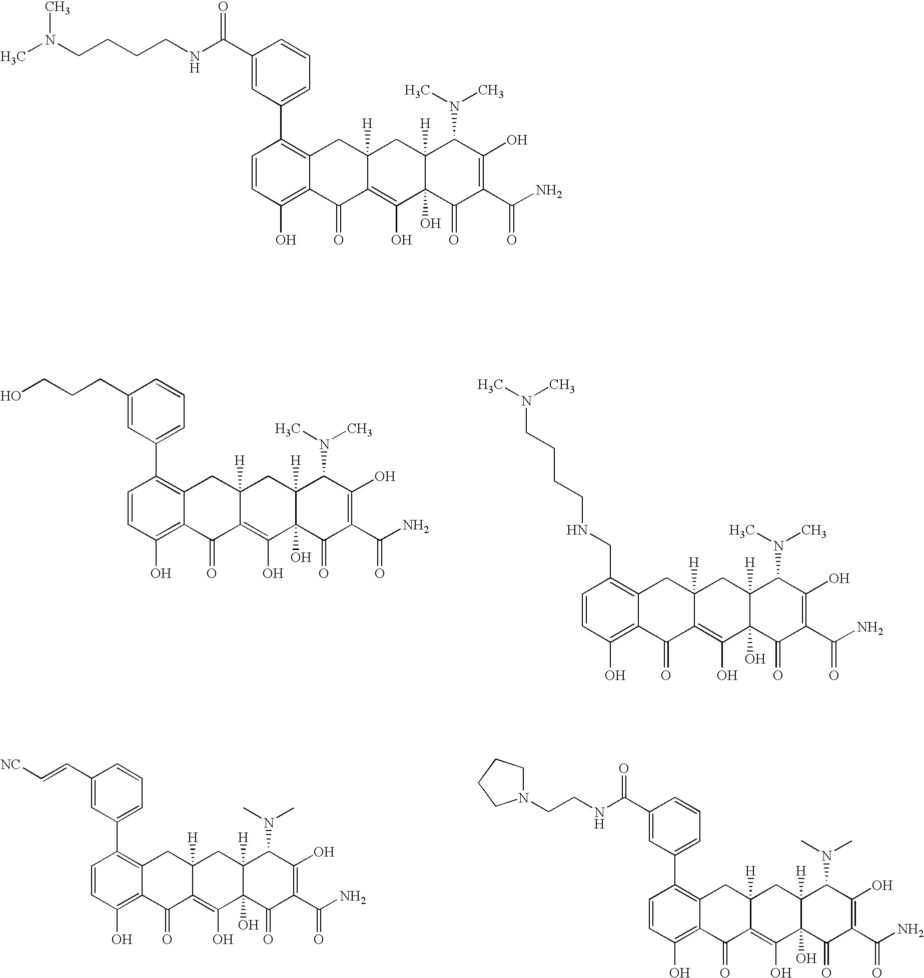 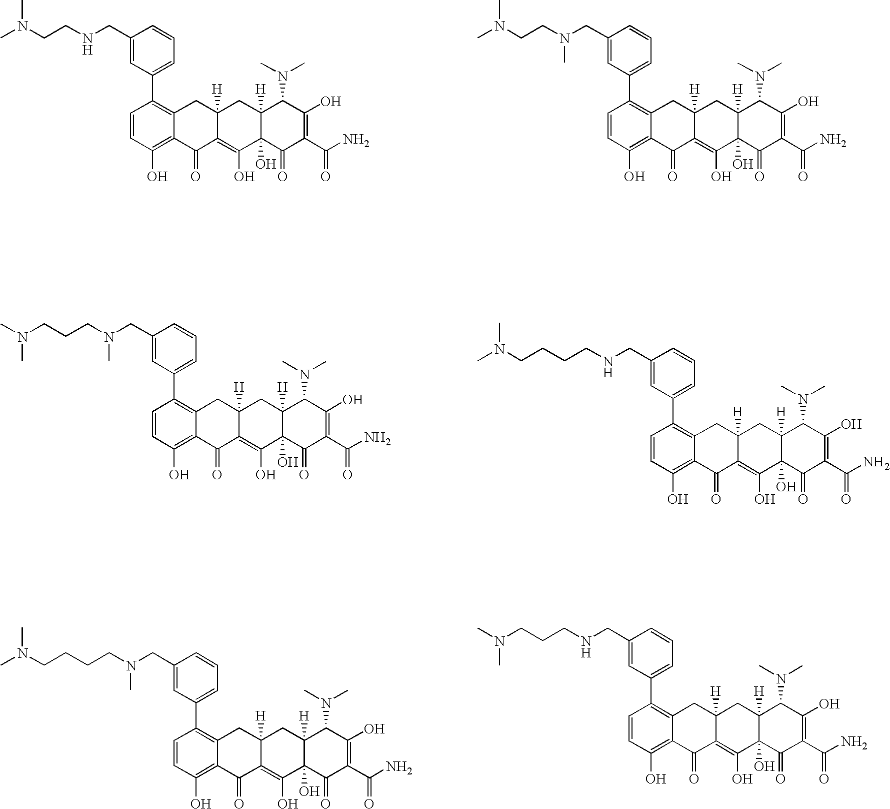 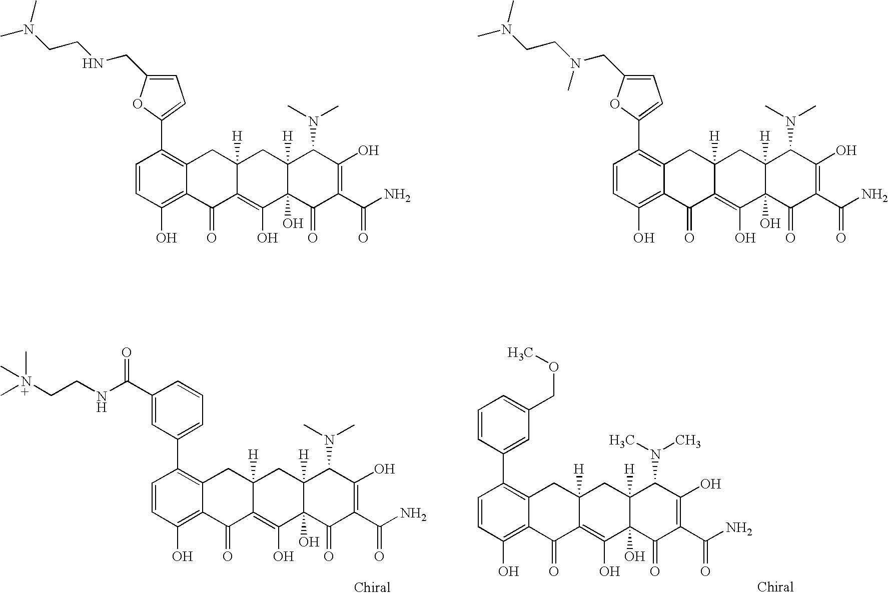 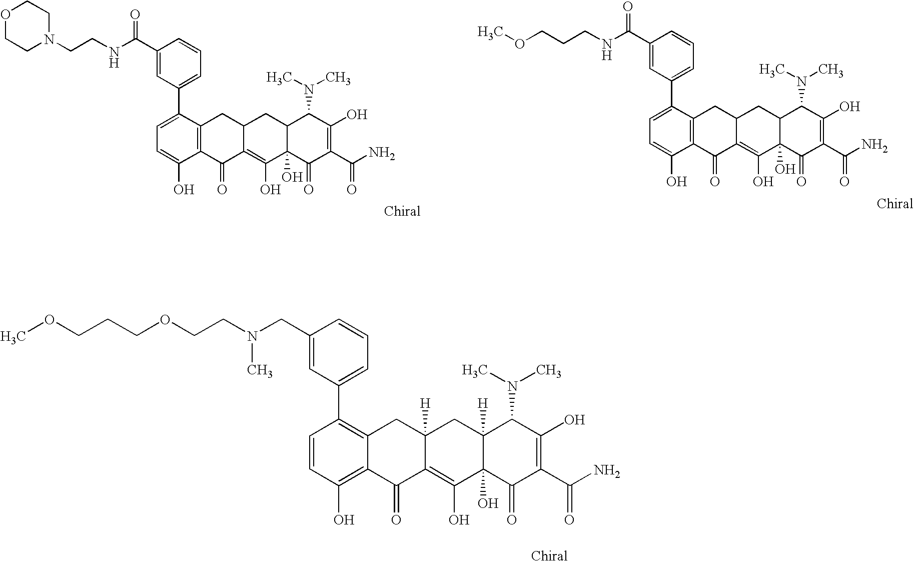 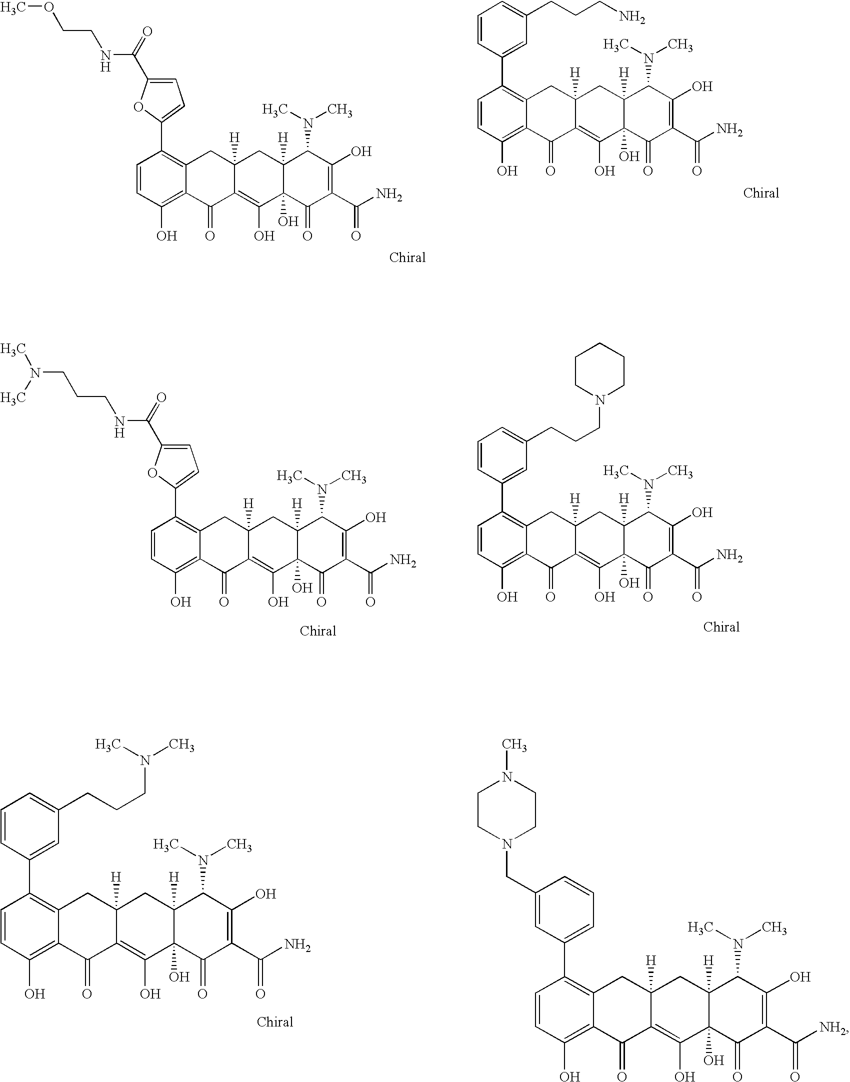
and pharmaceutically acceptable salts, esters and enantiomers thereof.

12. The method of claim 1, wherein said microorganism-associated infection is a bacterial infection.

13. The method of claim 12, wherein said bacterial infection is associated with E. coli.

14. The method of claim 12, wherein said bacterial infection is associated with S. aureus.

15. The method of claim 12, wherein said bacterial infection is associated with S. pneumonia.

16. The method of claim 12, wherein said bacterial infection is resistant to other tetracycline antibiotics.

17. The method of claim 1, wherein said subject is a human.

18. The method of claim 1, wherein said tetracycline compound is administered with a pharmaceutically acceptable carrier.

19. A pharmaceutical composition for the treatment of a microorganism-associated infection comprising a therapeutically effective amount of a tetracycline compound, wherein said tetracycline compound is of formula I: 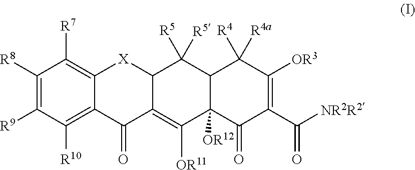
wherein
X is CHC(R¹³Y′Y), CR^{6′}R⁶, C═CR^{6′}R⁶, S, NR⁶, or O;
R², R^{2′}, R^{4′}, and R^{4″} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or a prodrug moiety;
R³, R^{4a}, R¹¹ and R¹² are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or a prodrug moiety;
R⁴ is NR^{4′} R^{4″}, hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or a prodrug moiety;
R⁵ and R^{5′} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic or a prodrug moiety;
R⁶ and R^{6′} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;
R⁷ is hydrogen, alkyl, alkenyl, alkynyl, acyl, hydroxyl, alkoxy, halogen, thioether, sulfinyl, sulfonyl, amino, cyano, nitro, carbonyl, oximyl, aryl, heterocyclic or —(CH₂)₀₋₃ (NR^{7c})₀₋₁C(═W′)WR^{7a};
R⁸ is hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or —(CH₂)₀₋₃(NR^{8c})₀₋₁C(=E′)ER^{8a};
R⁹ is hydrogen, alkyl, alkenyl, alkynyl, acyl, hydroxyl, alkoxy, halogen, thioether, sulfinyl, sulfonyl, amino, cyano, nitro, carbonyl, aryl, heterocyclic or —(CH₂)₀₋₃(NR^{9c})₀₋₁C(=Z′)ZR^{9a};
R¹⁰ is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;
R^{7a}, R^{7b}, R^{7c}, R^{7d}, R^{7e}, R^{7f}, R^{8a}, R^{8b}, R^{8c}, R^{8d}, R^{8e}, R^{8f}, R^{9a}, R^{9b}, R^{9c}, R^{9d}, R^{9e} and R^{9f} are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; R¹³ is hydrogen, alkyl, alkenyl, alkynyl, acyl, hydroxyl, alkoxy, halogen, thioether, sulfinyl, sulfonyl, amino, cyano, nitro, carbonyl, aryl or heterocyclic;
E is CR^{8d}R^{8e}, S, NR^{8b} or O;
E′ is O, NR^{8f}, or S;
W is CR^{7d}R^{7e}, S, NR^{7b} or O;
W′ is O, NR^{7f}, or S;
X is CHC(R¹³Y′Y), C═CR¹³Y, CR^{6′}R⁶, S, NR⁶ or O;
Z is CR^{9d}R^{9e}, S, NR^{9b} or O;
Z′ is O, S, or NR^{9f};
Y′ and Y are each independently hydrogen, alkyl, alkenyl, alkynyl, acyl, hydroxyl, alkoxy, halogen, thioether, sulfinyl, sulfonyl, amino, cyano, nitro, carbonyl, aryl or heterocyclic; and pharmaceutically acceptable salts, esters and enantiomers thereof;
and a pharmaceutically acceptable carrier.

20. The pharmaceutical composition of claim 19, wherein said microorganism-associated infection is a bacterial infection.
